(19) United States
(12) Patent Application Publication
(10) Pub. No.: US 2007/0287719 A1
(43) Pub. Date: Dec. 13, 2007

(54) Salts, Prodrugs and Formulations of 1-[5-(4-Amino-7-Isopropyl-7H-Pyrrolo[2,3-D]Pyrimidine-5-Carbonyl)-2-Methoxy-Phenyl]-3-(2,4-Dichloro-Phenyl)-Urea

(75) Inventors: Tracy L. Boyden, North Franklin, CT (US); Susan D. La Greca, Old Lyme, CT (US); Feng Guo, Colchester, CT (US); Douglas A. Lorenz, Bend, OR (US); Ravi M. Shanker, Groton, CT (US); Franzanne Vreeland, Groton, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/608,551

(22) Filed: Dec. 8, 2006

Related U.S. Application Data
(63) Continuation of application No. 11/078,739, filed on Mar. 11, 2005, now Pat. No. 7,163,942.
(60) Provisional application No. 60/749,070, filed on Dec. 9, 2005.

(51) Int. Cl. A61K 31/505

(52) U.S. Cl. 514/265.1; 544/280

(57) ABSTRACT

The invention relates to compounds of the formula 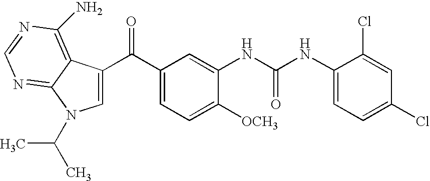 or a pharmaceutically acceptable salt, prodrug or hydrates thereof. The invention also relates to pharmaceutical compositions containing the compounds of formula 1 and to methods of treating hyperproliferative disorders in a mammal by administering the compounds of formula 1.

### BACKGROUND OF THE INVENTION

This invention relates to novel crystalline and non-crystalline forms and formulations of 1-[5-(4-amino-7-isopropyl-7H-pyrrolo[2,3-d]pyrimidine-5-carbonyl)-2-methoxy-phenyl]-3-(2,4-dichloro-phenyl)-urea. Such forms and formulations are useful in the treatment of hyperproliferative diseases, such as cancers, in mammals preferably a human. This invention also relates to methods of preparing such forms and formulations in the treatment of hyperproliferative diseases in mammals, especially humans.

1-[5-(4-Amino-7-isopropyl-7H-pyrrolo[2,3-d]pyrimidine-5-carbonyl)-2-methoxy-phenyl]-3-(2,4-dichloro-phenyl)-urea is a kinase inhibitor, more specifically a dual TIE-2 and Trk inhibitor, and is described in International Patent Publication WO 04/056830, published Jul. 8, 2004. 1-[5-(4-Amino-7-isopropyl-7H-pyrrolo[2,3-d]pyrimidine-5-carbonyl)-2-methoxy-phenyl]-3-(2,4-dichloro-phenyl)-urea is a hydrophobic molecule with extremely low aqueous solubility and low oral bioavailability when dosed conventionally. One aspect of the present invention is the discovery of formulations that ensure oral administration such that high bioavailabilities are achieved. More specifically, one aspect of the invention relates to solid amorphous dispersions, preferably spray dry dispersions, of 1-[5-(4-amino-7-isopropyl-7H-pyrrolo[2,3-d]pyrimidine-5-carbonyl)-2-methoxy-phenyl]-3-(2,4-dichloro-phenyl)-urea.

Solid amorphous dispersions, including spray dried dispersions, are well known in the art. Curatolo et al., EP 0 901 786 A2 disclose solid pharmaceutical dispersions with enhanced bioavailability using spray dried dispersions of a sparingly soluble drug and hydroxy propyl methyl cellulose acetate succinate. Nakamichi et al., U.S. Pat. No. 5,456,923 disclose an extrusion process for producing solid dispersions of sparingly soluble drugs and a variety of polymeric materials, such as hydroxy propyl methyl cellulose acetate succinate. Babcock et al., United States Patent Publication 2004/0156905, published Aug. 12, 2004, refers to pharmaceutical compositions of a drug in a semi-ordered state. Beyerinck et al., United States Patent Publication 2005/0031692, published Feb. 10, 2005, refers to further embodiments of solid amorphous dispersions, including spray drying processes. Other spray dry cases include International Patent Publication 03/063832, published Aug. 7, 2003.

As stated above, the active agent, 1-[5-(4-amino-7-isopropyl-7H -pyrrolo[2,3-d]pyrimidine-5-carbonyl)-2-methoxy-phenyl]-3-(2,4-dichloro-phenyl)-urea, is a kinase inhibitor possessing dual inhibitory activity against TIE-2 and Trk. Receptor tyrosine kinases are large enzymes that span the cell membrane and possess an extracellular binding domain for growth factors such as epidermal growth factor, a transmembrane domain, and an intracellular portion that functions as a kinase to phosphorylate specific tyrosine residue in proteins and hence to influence cell proliferation. The foregoing tyrosine kinases may be classified as growth factor receptor (e.g. TIE-2, TrkA, EGFR, PDGFR, FGFR and erbB2) or non-receptor (e.g. c-src and bcr-abl) kinases. It is known that such kinases are often aberrantly expressed in common human cancers such as breast cancer, gastrointestinal cancer such as colon, rectal or stomach cancer, leukemia, and ovarian, bronchial or pancreatic cancer. It is accepted in the scientific community that inhibitors of receptor tyrosine kinases, such as the compounds of the present invention, are useful as selective inhibitors of the growth of mammalian cancer cells.

Tie-2 (TEK) is a member of a family of endothelial cell specific receptor tyrosine kinases which are involved in critical angiogenic processes, such as vessel branching, sprouting, remodeling, maturation and stability. Tie-2 is the first mammalian receptor tyrosine kinase for which both agonist ligand(s) (e.g., Angiopoietin1 (“Ang1”), which stimulates receptor autophosphorylation and signal transduction), and antagonist ligand(s) (e.g., Angiopoietin2 (“Ang2”)), have been identified. Knock-out and transgenic manipulation of the expression of Tie-2 and its ligands indicates tight spatial and temporal control of Tie-2 signaling is essential for the proper development of new vasculature. The current model suggests that stimulation of Tie-2 kinase by the Ang1 ligand is directly involved in the branching, sprouting and outgrowth of new vessels, and recruitment and interaction of periendothelial support cells important in maintaining vessel integrity and inducing quiescence. The absence of Ang1 stimulation of Tie-2 or the inhibition of Tie-2 autophosphorylation by Ang2, which is produced at high levels at sites of vascular regression, may cause a loss in vascular structure and matrix contacts resulting in endothelial cell death, especially in the absence of growth/survival stimuli.

Trks are transmembrane proteins that contain an extracellular ligand binding domain, a transmembrane sequence, and a cytoplasmic tyrosine kinase domain. The trks comprise a family of structurally related proteins with preferential binding specificities for individual neurotrophins. TrkA, which is sometimes referred to as Trk, is a high-affinity receptor for NGF, but it can also mediate biological responses to NT-3 under particular conditions (Kaplan et al., Science, 252:554-558, 1991; Klein et al., Cell, 65, 189-197, 1991; Cordon-Cardo et al., Cell, 66:173-183, 1991). TrkB binds and mediates functions of BDNF, NT-3, and NT4/5 (Klein et al., Cell, 66:395-403, 1991; Squinto et al., Cell, 65:885-893, 1991; Klein et al., Neuron, 8:947-956, 1992). TrkC is relatively specific for NT-3 (Lamballe et al. Cell 66:967-979, 1991).

The Trk family of receptor tyrosine kinases is frequently expressed in lung, breast, pancreatic, and prostate cancers. See, Endocrinol., 141: 118, 2000; Cancer Res., 59: 2395, 1999; Clin. Cancer Res., 5: 2205, 1999; and Oncogene, 19: 3032, 2000. The tyrosine kinase activity of Trk is believed to promote the unregulated activation of cell proliferation machinery. Recent pre-clinical data suggests that Trk inhibitors suppress the growth of breast, pancreatic and prostate tumor xenografts. Furthermore, it is believed that Trk inhibition may be tolerated in cancer patients. It is also believed by those in the art that inhibitors of either TrkA or TrkB kinases have utility against some of the most common cancers, such as brain, melanoma, squamous cell, bladder, gastric, pancreatic, breast, head, neck, oesophageal, prostate, colorectal, lung, renal, kidney, ovarian, gynecological, and thyroid cancer. It is further believed that additional therapeutic uses of Trk inhibitors include pain, neurapthay and obesity.

### SUMMARY OF THE INVENTION

The present invention relates to novel crystalline and non-crystalline forms and formulations of 1-[5-(4-amino-7-isopropyl-7H-pyrrolo[2,3-d]pyrimidine-5-carbonyl)-2-methoxy-phenyl]-3-(2,4-dichloro-phenyl)-urea. 1-[5-(4-Amino-7-isopropyl-7H-pyrrolo[2,3-d]pyrimidine-5-carbonyl)-2-methoxy-phenyl]-3-(2,4-dichloro-phenyl)-urea has the formula

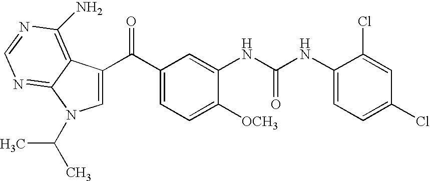

One specific preferred embodiment of the present invention relates to pharmaceutical compositions comprising a solid amorphous dispersion (more preferably a spray dry dispersion, SDD) of a form of 1-[5-(4-amino-7-isopropyl-7H-pyrrolo[2,3-d]pyrimidine-5-carbonyl)-2-methoxy-phenyl]-3-(2,4-dichloro-phenyl)-urea and a concentration-enhancing polymer.

As used herein, the terms “crystalline and non-crystalline forms,” “forms,” or any reference to the compound per se (unless otherwise specified), is meant to include any acceptable crystalline and non-crystalline freebase, solvate, hydrate, isomorph, polymorph, salt or prodrug of 1-[5-(4-amino-7-isopropyl-7H-pyrrolo[2,3-d]pyrimidine-5-carbonyl)-2-methoxy-phenyl]-3-(2,4-dichloro-phenyl)-urea. The most preferred form of 1-[5-(4-amino-7-isopropyl-7H-pyrrolo[2,3-d]pyrimidine-5-carbonyl)-2-methoxy-phenyl]-3-(2,4-dichloro-phenyl)-urea for formulation in the solid amorphous dispersions, more preferably the SDD, is its freebase.

The phrase “pharmaceutically acceptable salt(s)”, as used herein, unless otherwise indicated, includes acidic salts of 1-[5-(4-amino-7-isopropyl-7H-pyrrolo[2,3-d]pyrimidine-5-carbonyl)-2-methoxy-phenyl]-3-(2,4-dichloro-phenyl)-urea. 1-[5-(4-Amino-7-isopropyl-7H-pyrrolo[2,3-d]pyrimidine-5-carbonyl)-2-methoxy-phenyl]-3-(2,4-dichloro-phenyl)-urea is capable of forming a wide variety of salts with various inorganic and organic acids. The acids that may be used to prepare pharmaceutically acceptable acid addition salts of 1-[5-(4-Amino-7-isopropyl-7H-pyrrolo[2,3-d]pyrimidine-5-carbonyl)-2-methoxy-phenyl]-3-(2,4-dichloro-phenyl)-urea are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)]salts.

1-[5-(4-Amino-7-isopropyl-7H-pyrrolo[2,3-d]pyrimidine-5-carbonyl)-2-methoxy-phenyl]-3-(2,4-dichloro-phenyl)-urea may also exist as tautomers. This invention relates to the use of all such tautomers and mixtures thereof.

This invention also encompasses pharmaceutical compositions containing and methods of treating proliferative disorders or abnormal cell growth through administering prodrugs of 1-[5-(4-amino-7-isopropyl-7H-pyrrolo[2,3-d]pyrimidine-5-carbonyl)-2-methoxy-phenyl]-3-(2,4-dichloro-phenyl)-urea. Prodrugs include compounds wherein an amino acid residue, or a polypeptide chain of two or more (e.g., two, three or four) amino acid residues is covalently joined through an amide or ester bond to a free amino, hydroxy or carboxylic acid group of 1-[5-(4-amino-7-isopropyl-7H-pyrrolo[2,3-d]pyrimidine-5-carbonyl)-2-methoxy-phenyl]-3-(2,4-dichloro-phenyl)-urea. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem., 1996, 39, 10. Free amines can also be derivatized as amides, sulfonamides or phosphonamides. All of these prodrug moieties may incorporate groups including but not limited to ether, amine and carboxylic acid functionalities.

Active agent or “inhibitor” as used herein refers to any of the aforementioned forms of 1-[5-(4-amino-7-isopropyl-7H-pyrrolo[2,3-d]pyrimidine-5-carbonyl)-2-methoxy-phenyl]-3-(2,4-dichloro-phenyl)-urea that are pharmaceutically acceptable.

Another specific preferred embodiment of the invention relates to a pharmaceutical composition comprising a solid amorphous dispersion of 1-[5-(4-amino-7-isopropyl-7H-pyrrolo[2,3-d]pyrimidine-5-carbonyl)-2-methoxy-phenyl]-3-(2,4-dichloro-phenyl)-urea and a concentration-enhancing polymer, wherein said active agent comprises between 10 to 40 percent by weight of said solid amorphous dispersion, more preferably 15 to 30 percent most preferably 25 percent.

Another embodiment of the present invention is directed to solid amorphous dispersion, more preferably SDD, compositions wherein said concentration-enhancing polymer has at least one hydrophobic portion and at least one hydrophilic portion.

Another embodiment of the present invention is directed to solid amorphous dispersion, more preferably SDD, compositions wherein said concentration-enhancing polymer is a cellulosic polymer, more preferably a cellulosic polymer selected from the group consisting of ionizable cellulosic polymers, nonionizable cellulosic polymers, and vinyl copolymers and copolymers having substituents selected from the group consisting of hydroxyl, alkylacyloxy, and cyclicamido.

More specific embodiments of the present invention relate to compositions wherein said concentration-enhancing polymer is a cellulose polymer, more preferably a cellulose polymer selected from the group consisting of hydroxypropyl methyl cellulose acetate, hydroxypropyl methyl cellulose, hydroxypropyl cellulose, methyl cellulose, hydroxyethyl methyl cellulose, hydroxyethyl cellulose acetate, hydroxyethyl ethyl cellulose, hydroxypropyl methyl cellulose acetate succinate, cellulose acetate phthalate, hydroxypropyl methyl cellulose phthalate, methyl cellulose acetate phthalate, cellulose acetate trimellitate, hydroxypropyl cellulose acetate phthalate, cellulose acetate terephthalate, cellulose acetate isophthalate, and carboxy methyl ethyl cellulose.

A more specific embodiment of the invention is directed to solid amorphous dispersions comprising the concentration-enhancing polymer carboxymethyl ethyl cellulose. Another embodiment of the invention is directed to the concentration-enhancing polymer polyoxyethylene-polyoxypropylene copolymer.

One specific preferred embodiment of the solid amorphous dispersions invention is directed to the concentration-enhancing polymer hydroxypropyl methylcellulose acetate succinate (HPMCAS). An even more preferred embodiment of the solid amorphous dispersions invention is directed to the concentration-enhancing polymer hydroxypropyl methylcellulose acetate succinate (HPMCAS) where the succinate grade used is High Granular. Other preferred embodiments include the high fine analytical grade of succinate (HPMCAS-HF).

Even more preferred embodiments of the concentration-enhancing polymer hydroxypropyl methylcellulose acetate succinate (HPMCAS) solid amorphous dispersions, more preferably SDD, include those solid amorphous dispersions where the active agent comprises 10 to 40 percent by weight, more preferably 15 to 30 percent by weight, more preferably 25 percent by weight of the total solid amorphous dispersion, more preferably SDD, formulation.

Preferred embodiments of the concentration-enhancing polymer hydroxypropyl methylcellulose acetate succinate (HPMCAS) solid amorphous dispersions, more preferably SDD, include those solid amorphous dispersions wherein said 1-[5-(4-amino7-isopropyl-7H-pyrrolo[2,3-d]pyrimidine-5-carbonyl)-2-methoxy-phenyl]-3-(2,4-dichloro-phenyl)-urea in the dispersion is substantially amorphous and said dispersion is substantially homogeneous.

Preferred embodiments of the concentration-enhancing polymer hydroxypropyl methylcellulose acetate succinate (HPMCAS) solid amorphous dispersions, more preferably SDD, include those solid amorphous dispersions wherein said dispersion has a single glass transition temperature. Preferably wherein said transition temperature is from 110-120° C., more preferably 116° C.

Even more preferred embodiments include pharmaceutical compositions of said concentration-enhancing polymer hydroxypropyl methylcellulose acetate succinate (HPMCAS) solid amorphous dispersions, more preferably those solid amorphous dispersions where the active agent comprises 25 percent by weight of the total solid amorphous dispersion formulation, more preferably wherein said composition weighs no more than 1 gram per unit dose. Other most preferred embodiments of said pharmaceutical compositions of said concentration-enhancing polymer hydroxypropyl methylcellulose acetate succinate (HPMCAS) solid amorphous dispersions, include those wherein the active agent is 5, 25, 50, 100, 250 or 500 mg per unit dose.

Another specific preferred embodiment of the invention is directed to a concentration-enhancing polymer composition wherein said pharmaceutical composition comprises a solid amorphous dispersion of 1-[5-(4-amino-7-isopropyl-7H-pyrrolo[2,3-d]pyrimidine-5-carbonyl)-2-methoxy-phenyl]-3-(2,4-dichloro-phenyl)-urea and a concentration-enhancing polymer, wherein said concentration-enhancing polymer is present in said solid amorphous dispersion in a sufficient amount so that said composition provides concentration enhancement of said 1-[5-(4-amino-7-isopropyl-7H-pyrrolo[2,3-d]pyrimidine-5-carbonyl)-2-methoxy-phenyl]-3-(2,4-dichloro-phenyl)-urea in a use environment relative to a control composition consisting essentially of an equivalent amount of said 1-[5-(4-amino-7-isopropyl-7H-pyrrolo[2,3-d]pyrimidine-5-carbonyl)-2-methoxy-phenyl]-3-(2,4-dichloro-phenyl)-urea alone, more preferably wherein said concentration-enhancing polymer is hydroxypropyl methylcellulose acetate succinate.

As used herein, a “use environment” can be either the in vivo environment of the GI tract or blood plasma of a mammal, particularly a human, or the in vitro environment of a test solution, such as phosphate buffered saline (PBS) or Model Fasted Duodenal (MFD) solution.

The compositions of the present invention improve the aqueous concentration of 1-[5-(4-amino-7-isopropyl-7H-pyrolo[2,3-d]pyrimidine-5-carbonyl)-2-methoxy-phenyl]-3-(2,4-dichloro-phenyl)-urea relative to compositions that are free from concentration-enhancing polymer, by providing aqueous Cₘₐₓ concentration of 1-[5-(4-amino-7-isopropyl-7H-pyrrolo[2,3-d]pyrimidine-5-carbonyl)-2-methoxy-phenyl]-3-(2,4-dichloro-phenyl)-urea of at least about 10 fold that of control compositions that are free from the concentration-enhancing polymer. In fact, compositions of the present invention often exhibit enhancements of 5 to 500 fold, more preferably 15 to 100 fold, more preferably 20 to 50 fold, improvement in concentration relative to that of a control crystalline composition. More preferably said Cₘₐₓ enhancement is determined by a PBS or MFD dissolution test.

The compositions of the present invention improve the aqueous concentration of 1-[5-(4-amino-7-isopropyl-7H-pyrrolo[2,3-d]pyrimidine-5-carbonyl)-2-methoxy-phenyl]-3-(2,4-dichloro-phenyl)-urea relative to compositions that are free from concentration-enhancing polymer, by providing aqueous Cₘₐₓ concentration of 1-[5-(4-amino-7-isopropyl-7H-pyrrolo[2,3-d]pyrimidine-5-carbonyl)-2-methoxy-phenyl]-3-(2,4-dichloro-phenyl)-urea of between 10 and 500 μg/mL, more preferably between 50 and 100 μg/mL. More preferably said Cₘₐₓ enhancement is determined by a PBS or MFD dissolution test. Most preferably the enhancement Cₘₐₓ is determined according to the MFD dissolution test on a saturated solution.

In another embodiment of the invention, said composition provides in said use environment an area under the concentration versus time curve for any period of at least 90 minutes between the time of introduction into said use environment and about 270 minutes following introduction to the use environment that is at least about 10-fold, more preferably 50-fold, that of said control composition. More preferably said AUC₉₀ enhancement is determined by a PBS or MFD dissolution test.

In another embodiment of the invention, said composition provides in said use environment an area under the concentration versus time curve for any period of at least 90 minutes between the time of introduction into said use environment and about 270 minutes following introduction to the use environment that is between 500 and 10,000 μg×min/mL, more preferably between 1000 and 7000 μg×min/mL. More preferably said AUC₉₀ enhancement is determined by a PBS or MFD dissolution test. Most preferably the enhancement Cₘₐₓ is determined according to the MFD dissolution test on a saturated solution.

Another specific preferred embodiment of the invention is directed to a concentration-enhancing polymer composition wherein said composition when administered at least once during a 24 hour period in an oral dosage form of between 5 mg and 500 mg, more preferably 50, 100 or 200 mg, of active agent to a human, has a Cₘₐₓ plasma level as determined in a fasting rat at a dose of 100 mg active agent per kg, between 20000 ng base/ml to 1000 ng base/ml, more preferably 15000 ng base/ml and 3000 ng base/ml, more preferably 10000 ng base/mL and 5000 ng base/mL over said 24 hour period.

Concentration-enhancing polymer composition as used herein refers to a pharmaceutical composition comprising a concentration-enhancing polymer and active agent, optionally containing additional pharmaceutically acceptable excipients.

Another specific preferred embodiment of the invention is directed to a concentration-enhancing polymer composition wherein said composition when administered at least once during a 24 hour period in an oral dosage form of between 5 mg and 500 mg, more preferably 50, 100 or 200 mg, of active agent to a human, has a Cₘₐₓ plasma level as determined in a fasting dog at a dose of 30 mg active agent per kg, between 15000 ng base/ml to 1000 ng base/ml, more preferably 10000 ng base/ml and 1000 ng base/ml, more preferably 5000 ng base/mL and 1110 ng base/mL over said 24 hour period.

Another specific preferred embodiment of the invention is directed to a concentration-enhancing polymer composition wherein said composition when administered at least once during a 24 hour period in an oral dosage form of between 5 mg and 500 mg, more preferably 50, 100 or 200 mg, of active agent to a human, has a AUC₀₋₂₄ plasma level as determined in a fasting rat at a dose of 100 mg active agent per kg, between 150000 ng base×hr/mL and 5000 ng base×hr/mL, more preferably between 100000 ng base×hr/mL and 40000 ng base×hr/mL, more preferably between 90000 ng base×hr/mL and 60000 ng base×hr/mL.

Another specific preferred embodiment of the invention is directed to a concentration-enhancing polymer composition wherein said composition when administered at least once during a 24 hour period in an oral dosage form of between 5 mg and 500 mg, more preferably 50, 100 or 200 mg, of active agent to a human, has a AUC₀₋₂₄ plasma level as determined in a fasting dog at a dose of 30 mg active agent per kg, between 100000 ng base×hr/mL and 8000 ng base×hr/mL, more preferably between 80000 ng base×hr/mL and 10000 ng base×hr/mL, more preferably between 50000 ng base×hr/mL and 10000 ng base×hr/mL.

Another specific preferred embodiment of the invention is directed to a concentration-enhancing polymer composition wherein said composition when administered at least once during a 24 hour period in an oral dosage form of between 5 mg and 500 mg, more preferably 50, 100 or 200 mg, of active agent to a human, has a Tₘₐₓ plasma level as determined in a fasting rat at a dose of 100 mg active agent per kg, between 3 hours and 30 minutes, more preferably between 2.5 hours and 1 hour, more preferably less than 2 hours.

Another specific preferred embodiment of the invention is directed to a concentration-enhancing polymer composition wherein said composition when administered at least once during a 24 hour period in an oral dosage form of between 5 mg, and 500 mg, more preferably 50, 100 or 200 mg, of active agent to a human, has a Tₘₐₓ plasma level as determined in a fasting dog at a dose of 30 mg active agent per kg, between 3 hours and 30 minutes, more preferably between 2.5 hours and 1 hour, more preferably less than 2 hours.

In another embodiment of the invention, said solid amorphous dispersion is mixed with additional concentration-enhancing polymer.

In another embodiment of the invention, said concentration-enhancing polymer comprises a blend of polymers.

In another embodiment of the invention, said concentration-enhancing polymer has at least one hydrophobic portion and at least one hydrophilic portion.

In another embodiment of the invention, said concentration-enhancing polymer is selected from the group consisting of ionizable cellulosic polymers, nonionizable cellulosic polymers, and vinyl copolymers and copolymers having substituents selected from the group consisting of hydroxyl, alkylacyloxy, and cyclicamido.

In another embodiment of the invention, said concentration-enhancing polymer is selected from the group consisting of hydroxypropyl methyl cellulose acetate, hydroxypropyl methyl cellulose, hydroxypropyl cellulose, methyl cellulose, hydroxyethyl methyl cellulose, hydroxyethyl cellulose acetate, hydroxyethyl ethyl cellulose, hydroxypropyl methyl cellulose acetate succinate, cellulose acetate phthalate, hydroxypropyl methyl cellulose phthalate, methyl cellulose acetate phthalate, cellulose acetate trimellitate, hydroxypropyl cellulose acetate phthalate, cellulose acetate terephthalate, cellulose acetate isophthalate, and carboxy methyl ethyl cellulose.

In another embodiment of the invention, said solid amorphous dispersion is formulated in a tablet.

In another embodiment of the invention, said solid amorphous dispersion includes a disintegrant such as sodium starch glycolate, sodium alginate, carboxy methyl cellulose sodium, methylcellulose, and croscarmellose sodium.

In another embodiment of the invention, said solid amorphous dispersion includes a binder such as methyl cellulose, microcrystalline cellulose, starch, and gums such as guar gum, and tragacanth.

In another embodiment of the invention, said solid amorphous dispersion includes a lubricant such as magnesium stearate and calcium stearate.

In another embodiment of the invention, said solid amorphous dispersion is formulated in a capsule.

Another specific embodiment of the present invention relates to a method of forming the solid amorphous dispersion by solvent processing. According to this embodiment, a solution is formed comprising the active agent and a concentration-enhancing polymer dissolved in a common solvent. Solvent is then rapidly removed from the solution to form a solid amorphous dispersion of the active agent and the concentration-enhancing polymer.

Another embodiment of the present invention is directed to a method for forming pharmaceutical compositions by melt extrusion. The active agent and a concentration-enhancing polymer are fed to an extruder. The active agent and polymer are extruded through the extruder and then rapidly solidified to form a solid amorphous dispersion comprising the active agent and the concentration-enhancing polymer.

Another embodiment of the present invention is directed to a method for forming pharmaceutical compositions by melt congealing. A molten mixture comprising the active agent and a concentration-enhancing polymer is formed. The mixture is then cooled to form a solid amorphous dispersion comprising the active agent and the concentration-enhancing polymer.

Other embodiments of the present invention relate to salts and polymorphs of the 1-[5-(4amino-7-isopropyl-7H-pyrrolo[2,3-d]pyrimidine-5-carbonyl)-2-methoxy-phenyl]-3-(2,4-dichloro-phenyl)-urea.

Specific embodiments include the phosphate, mesylate, besylate, tosylate and hydrochloride salts of 1-[5-(4-amino-7-isopropyl-7H-pyrrrolo[2,3-d]pyrimidine-5-carbonyl)-2-methoxy-phenyl]-3-(2,4-dichloro-phenyl)-urea.

Yet other embodiments of the present invention relate to pharmaceutical compositions of said salts and polymorphs of 1-[5-(4-amino-7-isopropyl-7H-pyrrolo[2,3-d]pyrimidine-5-carbonyl)-2-methoxy-phenyl]-3-(2,4-dichloro-phenyl)-urea.

Yet other embodiments of the present invention relate to capsules or tablets comprising pharmaceutically acceptable compositions of the aforesaid forms and spray dry dispersions. The compositions may be dosed in a variety of dosage units, including both immediate release and controlled release dosage units, the latter including both delayed and sustained release tablets or capsules. The composition may include blends of polymers, and may further include other excipients that further improve the bioavailability of 1-[5-(4-amino-7-isopropyl-7H-pyrrolo[2,3-d]pyrimidine-5-carbonyl)-2-methoxy-phenyl]-3-(2,4-dichloro-phenyl)-urea.

The present invention also relates to a method for the treatment of abnormal cell growth in a mammal, including a human, comprising administering to same mammal in need of such treatment an amount of a salt, polymorph or spray dry dispersion of 1-[5-(4-amino-7-isopropyl-7H-pyrrolo[2,3-d]pyrimidine-5-carbonyl)-2-methoxy-phenyl]-3-(2,4-dichloro-phenyl)-urea, that is effective in treating abnormal cell growth.

The present invention also relates to methods of administering the compositions described above.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; and (4) any tumors that proliferate by receptor tyrosine kinases.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

In one embodiment of this method, the abnormal cell growth is cancer, including, but not limited to, mesothelioma, hepatobilliary cancers (hepatic and billiary duct), a primary or secondary CNS tumor, a primary or secondary brain tumor (including pituitary tumors, astrocytomas, meningiomas and medulloblastomas), lung cancer (NSCLC and SCLC), bone cancer, pancreatic cancer, skin cancer, cancer of the head or neck, cutaneous or intraocular melanoma, ovarian cancer, colon cancer, rectal cancer, liver cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, gastrointestinal stromal tumor (GIST), pancreatic endocrine tumors (such as pheochromocytoma, insulinoma, vasoactive intestinal peptide tumor, islet cell tumor and glucagonoma), carcinoid tumors, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, tumors of the blood vessels (including benign and malignant tumors such as hemangiomas, hemangiosarcomas, hemangioblastomas and lobular capillary hemangiomas) or a combination of one or more of the foregoing cancers.

Another more specific embodiment of the present invention is directed to a cancer selected from lung cancer (NSCLC and SCLC), cancer of the head or neck, ovarian cancer, colon cancer, rectal cancer, cancer of the anal region, stomach cancer, breast cancer, cancer of the kidney or ureter, renal cell carcinoma, carcinoma of the renal pelvis, neoplasms of the central nervous system (CNS), primary CNS lymphoma, non-Hodgkins's lymphoma, spinal axis tumors, or a combination of one or more of the foregoing cancers.

In another more specific embodiment of the present invention the cancer is selected from lung cancer (NSCLC and SCLC), breast cancer, ovarian cancer, colon cancer, rectal cancer, cancer of the anal region, or a combination of one or more of the foregoing cancers.

In another embodiment of the present invention, said abnormal cell growth is a benign proliferative disease, including, but not limited to, psoriasis, benign prostatic hypertrophy, restinosis, synovial, proliferation disorder, retinopathy or other neovascular disorders of the eye, pulmonary hypertension or mobilization of TIE-2 positive stem cells from bone marrow for use in reconstituting normal cells of any tissue.

This invention also relates to a method for the treatment of abnormal cell growth in a mammal in need of such treatment, which comprises administering to said mammal an amount of 1-[5-(4-amino-7-isopropyl-7H-pyrrolo[2,3-d]pyrimidine-5-carbonyl)-2-methoxy-phenyl]-3-(2,4-dichloro-phenyl)-urea (including each of the aforementioned forms and formulations including each of said hydrates, solvates and polymorphs or pharmaceutically acceptable salts thereof), in combination with one or more (preferable one to three) anti-cancer agents selected from the group consisting of traditional anticancer agents (such as DNA binding agents, mitotic inhibitors, alkylating agents, anti-metabolites, intercalating antibiotics, topoisomerase inhibitors and microtubulin inhibitors), statins, radiation, angiogenesis inhibitors, signal transduction inhibitors, cell cycle inhibitors, telomerase inhibitors, biological response modifiers (such as antibodies, immunotherapy and peptide mimics), anti-hormones, anti-androgens, gene silencing agents, gene activating agents and anti-vascular agents, wherein the amounts of 1-[5-4-amino-7-isopropyl-7H-pyrrolo[2,3-d]pyrimidine-5-carbonyl)-2-methoxy-phenyl]-3-(2,4-dichloro-phenyl)-urea together with the amounts of the combination anticancer agents is effective in treating abnormal cell growth.

The invention also relates to a method for the treatment of a hyperproliferative disorder in a mammal in need of such treatment, comprising administering to said mammal an amount of 1-[5-(4-amino-7-isopropyl-7H-pyrrolo[2,3-d]pyrimidine-5-carbonyl)-2-methoxy-phenyl]-3-(2,4-dichloro-phenyl)-urea (including all forms and formulations thereof and each of aforesaid hydrates, solvates and polymorphs thereof or pharmaceutically acceptable salts thereof), in combination with an anti-cancer agent selected from the group consisting of traditional anticancer agents (such as DNA binding agents, mitotic inhibitors, alkylating agents, anti-metabolites, intercalating antibiotics, topoisomerase inhibitors and microtubulin inhibitors), statins, radiation, angiogenesis inhibitors, signal transduction inhibitors, cell cycle inhibitors, telomerase inhibitors, biological response modifiers (such as antibodies, immunotherapy and peptide mimics), hormones anti-hormones, anti-androgens, gene silencing agents, gene activating agents and anti-vascular agents, wherein the amounts of 1-[5-(4-amino-7-isopropyl-7H-pyrrolo[2,3-d]pyrimidine-5-carbonyl)-2-methoxy-phenyl]-3-(2,4-dichloro-phenyl)-urea together with the amounts of the combination anticancer agents is effective in treating said hyperproliferative disorder.

This invention also relates to a pharmaceutical composition for the treatment of abnormal cell growth in a mammal, including a human, comprising an amount of 1-[5-(4-amino-7-isopropyl-7H-pyrrolo[2,3-d]pyrimidine-5-carbonyl)-2-methoxy-phenyl]-3-(2,4-dichloro-phenyl)-urea, as defined above (including hydrates, solvates and polymorphs of said compound or pharmaceutically acceptable salts thereof), that is effective in treating abnormal cell growth, and a pharmaceutically acceptable carrier. In one embodiment of this composition, the abnormal cell growth is cancer, including, but not limited to, mesothelioma, hepatobilliary cancer (hepatic and billiary duct), a primary or secondary CNS tumor, a primary or secondary brain tumor (including pituitary tumors, astrocytomas, meningiomas and medulloblastomas), lung cancer (NSCLC and SCLC), bone cancer, pancreatic cancer, skin cancer, cancer of the head or neck, cutaneous or intraocular melanoma, ovarian cancer, colon cancer, rectal cancer, liver cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, gastrointestinal stromal tumor (GIST), pancreatic endocrine tumors (such as pheochromocytoma, insulinoma, vasoactive intestinal peptide tumor, islet cell tumor and glucagonoma), carcinoid tumors, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, tumors of the blood vessels (including benign and malignant tumors such as hemangiomas, hemangiosarcomas, hemangioblastomas and lobular capillary hemangiomas) or a combination of one or more of the foregoing cancers.

The invention also relates to a pharmaceutical composition for the treatment of abnormal cell growth in a mammal, including a human, which comprises an amount of 1-[5-(4-amino-7-isopropyl-7H-pyrrolo[2,3-d]pyrimidine-5-carbonyl)-2-methoxy-phenyl]-3-(2,4-dichloro-phenyl)-urea, as defined above (including hydrates, solvates and polymorphs of said compound or pharmaceutically acceptable salts thereof), in combination with one or more (preferably one to three) anti-cancer agent selected from the group consisting of traditional anticancer agents (such as DNA binding agents, mitotic inhibitors, alkylating agents, anti-metabolites, intercalating antibiotics, topoisomerase inhibitors and microtubulin inhibitors), statins, radiation, angiogenesis inhibitors, signal transduction inhibitors, cell cycle inhibitors, telomerase inhibitors, biological response modifiers, hormones, anti-hormones, anti-androgens gene silencing agents, gene activating agents and anti-vascular agents and a pharmaceutically acceptable carrier, wherein the amounts of the active agent and the combination anti-cancer agents when taken as a whole is therapeutically effective for treating said abnormal cell growth.

In one embodiment of the present invention the anti-cancer agent used in conjunction with 1-[5-(4-amino-7-isopropyl-7H-pyrrolo[2,3-d]pyrmidine-5-carbonyl)-2-methoxy-phenyl]-3-(2,4-dichloro-phenyl)-urea and pharmaceutical compositions described herein is an anti-angiogenesis agent.

A more specific embodiment of the present invention includes combinations of 1-[5-(4-amino-7-isopropyl-7H-pyrrolo[2,3-d]pyrimidine-5-carbonyl)-2-methoxy-phenyl]-3-(2,4-dichloro-phenyl)-urea with anti-angiogenesis agents selected from VEGF inhibitors. VEGFR inhibitors, TIE-2 inhibitors, PDGFR inhibitors, angiopoetin inhibitors, PKCβ inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors.

Preferred VEGF inhibitors, include for example, Avastin (bevacizumab), an anti-VEGF monoclonal antibody of Genentech, Inc. of South San Francisco, Calif.

Additional VEGF inhibitors include CP-547,632 (Pfizer Inc., NY, USA), AG13736 (Pfizer Inc.), Vandetanib (Zactima), sorafenib (Bayer/Onyx), AEE788 (Novartis), AZD-2171, VEGF Trap (Regeneron,/Aventis), vatalanib (also known as PTK-787, ZK-222584; Novartis & Schering AG as described in U.S. Pat. No. 6,258,812), Macugen (pegaptanib octasodium, NX-1838, EYE-001, Pfizer Inc./Gilead/Eyetech), IM862 (Cytran Inc. of Kirkland, Wash., USA); Neovastat (Aeterna); and Angiozyme (a synthetic ribozyme that cleaves mRNA producing VEGF1) and combinations thereof. VEGF inhibitors useful in the practice of the present invention are disclosed in U.S. Pat. No. 6,534,524 and 6,235,764, both of which are incorporated in their entirety for all purposed.

Particularly preferred VEGF inhibitors include CP-547,632, AG-13736, AG-28262, Vatalanib, sorafenib, Macugen and combinations thereof.

Additional VEGF inhibitors are described in, for example in U.S. Pat. No. 6,492,383, issued Dec. 10, 2002, U.S. Pat. No. 6,235,764 issued May 22, 2001, U.S. Pat. No. 6,177,401 issued Jan. 23, 2001, U.S. Pat. No. 6,395,734 issued May 28, 2002, U.S. Pat. No. 6,534,524 (discloses AG13736) issued Mar. 18, 2003, U.S. Pat. No. 5,834,504 issued Nov. 10, 1998, U.S. Pat. No. 6,316,429 issued Nov. 13, 2001, U.S. Pat. No. 5,883,113 issued Mar. 16, 1999, U.S. Pat. No. 5,886,020 issued Mar. 23, 1999, U.S. Pat. No. 5,792,783 issued Aug. 11, 1998, U.S. Pat. No. 6,653,303 issued Nov. 25, 2003, WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety.

PDGFr inhibitors include but not limited to those disclosed in International Patent Publication number WO 01/40217, published Jun. 7, 2001 and International Patent Publication number WO 2004/020431, published Mar. 11, 2004, the contents of which are incorporated in their entirety for all purposes.

Preferred PDGFr inhibitors include Pfizer's CP-673,451 and CP-868,596 and their pharmaceutically acceptable salts.

TIE-2 inhibitors include GlaxoSmithKline's benzimidazoles and pyridines including GW-697465A such as described in International Patent Publications WO 02/044156 published Jun. 6, 2002, WO 03/066601 published Aug. 14, 2003, WO 03/074515 published Sep. 12, 2003, WO 03/022852 published Mar. 20, 2003 and WO 01/37835 published May 31, 2001. Other TIE-2 inhibitors include Regeneron's biologicals such as those described in International Patent Publication WO 09/611269 published Apr. 18, 1996, Amgen's AMG-386, and Abbott's pyrrolopyrimidines such as A-422885 and BSF-466895 described in International Patent Publications WO 09/955335, WO 09/917770, WO 00/1075139, WO 00/027822, WO 00/017203 and WO 00/017202.

In another more specific embodiment of the present invention the anti-cancer agent used in conjunction with 1-[5-(4-amino-7-isopropyl-7H-pyrrolo[2,3-d]pyrimidine-5-carbonyl)-2-methoxy-phenyl]-3-(2,4-dichloro-phenyl)-urea and pharmaceutical compositions described herein is where the anti-angiogenesis agent is a protein kinase C β such as enzastaurin, midostaurin, perifosine, staurosporine derivative (such as RO318425, RO317549, RO318830 or RO318220 (Roche)), teprenone (Selbex) and UCN-01 (Kyowa Hakko)

Examples of useful COX-II inhibitors which can be used in conjunction with 1-[5-(4-amino-7-isopropyl-7H-pyrrolo[2,3-d]pyrimidine-5-carbonyl)-2-methoxy-phenyl]-3-(2,4-dichloro-phenyl)-urea and pharmaceutical compositions described herein include CELEBREX (celecoxib), parecoxib, deracoxib, ABT-963, COX-189 (Lumiracoxib), BMS 347070, RS 57067, NS-398, Bextra (valdecoxib), Vioxx (rofecoxib), SD-8381, 4-methyl-2-(3,4-dimethylphenyl)-1-(4-sulfamoyl-phenyl)-1H-pyrrole, 2-(4-ethoxyphenyl)-4-methyl-1-(4-sulfamoylphenyl)-1H-pyrrole, T-614, JTE-522, S-2474, SVT-2016, CT-3, SC-58125 and Arcoxia (etoricoxib). Additionally, COX-II inhibitors are disclosed in U.S. patent application Ser. Nos. 10/801,446 and 10/801,429, the contents of which are incorporated in their entirety for all purposes.

In one specific embodiment of particular interest the anti-tumor agent is celecoxib as disclosed in U.S. Pat. No. 5,466,823, the contents of which are incorporated by reference in its entirety for all purposes.

In another embodiment the anti-tumor agent is deracoxib as disclosed in U.S. Pat. No. 5,521,207, the contents of which are incorporated by reference in its entirety for all purposes.

Other useful anti-angiogenic inhibitors used in conjunction with 1-[5-(4-amino-7-isopropyl-7H-pyrrolo[2,3-d]pyrimidine-5-carbonyl)-2-methoxy-phenyl]-3-(2,4-dichloro-phenyl)-urea and pharmaceutical compositions described herein include aspirin, and non-steroidal anti-inflammatory drugs (NSAIDs) which nonselectively inhibit the enzymes that make prostaglandins (cyclooxygenase I and II), resulting in lower levels of prostaglandins. Such agents include, but are not limited to, Aposyn (exisulind), Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac, (Toradol), Oxaprozin (Daypro) and combinations thereof.

Preferred nonselective cyclooxygenase inhibitors include ibuprofen (Motrin), nuprin, naproxen (Aleve), indomethacin (Indocin), nabumetone (Relafen) and combinations thereof.

MMP inhibitors include ABT-510 (Abbott), ABT 518 (Abbott), Apratastat (Amgen), AZD 8955 (AstraZeneca), Neovostat (AE-941), COL 3 (CollaGenex Pharmaceuticals), doxycycline hyclate, MPC 2130 (Myriad) and PCK 3145 (Procyon).

Other anti-angiogenic compounds include acitretin, angiostatin, aplidine, cilengtide, COL-3, combretastatin A-4, endostatin, fenretinide, halofuginone, Panzem (2-methoxyestradiol), PF03446962 (ALK-1 inhibitor), rebimastat, removab, Revlimid, squalamine, thalidomide, ukrain, Vitaxin (alpha-v/beta-3 integrin), and zoledronic acid.

In another embodiment the anti-cancer agent is a so called signal transduction inhibitor. Such inhibitors include small molecules, antibodies, and antisense molecules. Signal transduction inhibitors include kinase inhibitors, such as tyrosine kinase inhibitors, serine/threonine kinase inhibitors. Such inhibitors may be antibodies or small molecule inhibitors. More specifically signal transduction inhibitors include farnesyl protein transferase inhibitors, EGF inhibitor, ErbB-1 (EGFR), ErbB-2, pan erb, IGF1R inhibitors, MEK, c-Kit inhibitors, FLT-3 inhibitors, K-Ras inhibitors, PI3 kinase inhibitors, JAK inhibitors, STAT inhibitors, Raf kinase inhibitors, Akt inhibitors, mTOR inhibitor, P70S6 kinase inhibitors and inhibitors of the WNT pathway and so called multi-targeted kinase inhibitors.

In another embodiment the anti-cancer signal transduction inhibitor is a farnesyl protein transferase inhibitor. Farnesyl protein transferase inhibitors include the compounds disclosed and claimed in U.S. Pat. No. 6,194,438, issued Feb. 27, 2002; U.S. Pat. No. 6,258,824, issued Jul. 10, 2001; U.S. Pat. No. 6,586,447, issued Jul. 1, 2003; U.S. Pat. No. 6,071,935, issued Jun. 6, 2000; and U.S. Pat. No. 6,150,377, issued Nov. 21, 2000. Other farnesyl protein transferase inhibitors include AZD-3409 (AstraZeneca), BMS-214662 (Bristol-Myers Squibb), Lonafarnib (Sarasar) and RPR-115135 (Sanofi-Aventis). Each of the foregoing patent applications and provisional patent applications is herein incorporated by reference in their entirety.

In another embodiment the anti-cancer signal transduction inhibitor is a GARF inhibitor. Preferred GARF inhibitors (glycinamide ribonucleotide formyltransferse inhibitors) include Pfizer's AG-2037 (pelitrexol) and its pharmaceutically acceptable salts. GARF inhibitors useful in the practice of the present invention are disclosed in U.S. Pat. No. 5,608,082 which is incorporated in its entirety for all purposed.

In another embodiment the anti-cancer signal transduction inhibitors used in conjunction with 1-[5-(4-amino-7-isopropyl-7H-pyrrolo[2,3-d]pyrimidine-5-carbonyl)-2-methoxy-phenyl]-3-(2,4-dichloro-phenyl)-urea and pharmaceutical compositions described herein include ErbB-1 (EGFr) inhibitors such as Iressa (gefitinib, AstraZeneca), Tarceva (erlotinib or OSI-774, OSI Pharmaceuticals Inc.), Erbitux (cetuximab, Imclone Pharmaceuticals, Inc.), Matuzumab (Merck AG), Nimotuzumab, Panitumumab (Abgenix/Amgen), Vandetanib, hR3 (York Medical and Center for Molecular Immunology), TP-38 (IVAX), EGFR fusion protein, EGF-vaccine, anti-EGFr immunoliposomes (Hermes Biosciences Inc.) and combinations thereof.

Preferred EGFr inhibitors include Iressa (gefitinib), Erbotix, Tarceva and combinations thereof.

In another embodiment the anti-cancer signal transduction inhibitor is selected from pan erb receptor inhibitors or ErbB2 receptor inhibitors, such as CP-724,714, PF-299804, CI-1033 (canertinib, Pfizer, Inc.) Herceptin (trastuzumab, Genentech Inc.), Omnitarg (2C4, pertuzumab, Genentech Inc.), TAK-165 (Takeda), GW-572016 (lapatinib, GlaxoSmithKline), Pelitinib (EKB-569 Wyeth), BMS-599626, PKI-166 (Novartis), dHER2 (HER2 Vaccine, Corixa and GlaxoSmithKline), Osidem (IDM-1), APC8024 (HER2 Vaccine, Dendreon) anti-HER2/neu bispecific antibody (Decof Cancer Center), B7.her2.IgG3 (Agensys), AS HER2 (Research Institute for Rad Biology & Medicine), trifunctional bispecific antibodies (University of Munich) and mAB AR-209 (Aronex Pharmaceuticals Inc) and mAB 2B-1 (Chiron) and combinations thereof.

Preferred erb selective anti-tumor agents include Herceptin, TAK-165, CP-724,714, ABX-YEGF, HER3 and combinations thereof.

Preferred pan erb receptor inhibitors include GW572016, PF-299804, Pelitinib, and Omnitarg and combinations thereof.

Additional erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995). U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Pat. Nos. 6,465,449, and 6,284,764, and International Application No. WO 2001/98277 each of which are herein incorporated by reference in their entirety.

Various other compounds, such as styrene derivatives, have also been shown to possess tyrosine kinase inhibitory properties, and some of tyrosine kinase inhibitors have been identified as erbB2 receptor inhibitors. Other erbB2 inhibitors are described in European patent publications EP 566,226 A1 (published Oct. 20, 1993), EP 602,851 A1 (published Jun. 22, 1994), EP 635,507 A1 (published Jan. 25, 1995), EP 635,498 A1 (published Jan. 25, 1995), and EP 520,722 A1 (published Dec. 30, 1992). These publications refer to certain bicyclic derivatives, in particular quinazoline derivatives possessing anti-cancer properties that result from their tyrosine kinase inhibitory properties. Also, World Patent Application WO 92/20842 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are World Patent Application WO00/44728 (published Aug. 3, 2000), EP 1029853A1 (published Aug. 23, 2000), and WO01/98277 (published Dec. 12, 2001) all of which are incorporated herein by reference in their entirety.

In another embodiment the anti-cancer signal transduction inhibitor is an IGF1R inhibitor. Specific IGF1R antibodies (such as CP-751871) that can be used in the present invention include those described in International Patent Application No. WO 2002/053596, which is herein incorporated by reference in its entirety.

In another embodiment the anti-cancer signal transduction inhibitor is a MEK inhibitor. MEK inhibitors include Pfizer's MEK1/2 inhibitor PD325901, Array Biopharm's MEK inhibitor ARRY-142886, and combinations thereof.

In another embodiment the anti-cancer signal transduction inhibitor is an mTOR inhibitor. mTOR inhibitors include everolimus (RAD001, Novartis), zotarolimus, temsirolimus (CCI-779, Wyeth), AP 23573 (Ariad), AP23675, Ap23841, TAFA 93, rapamycin (sirolimus) and combinations thereof.

In another embodiment the anti-cancer signal transduction inhibitor is an Aurora 2 inhibitor such as VX-680 and derivatives thereof (Vertex), R 763 and derivatives thereof (Rigel) and ZM 447439 and AZD 1152 (AstraZeneca), or a Checkpoint kinase 1/2 inhibitors such as XL844 (Exilixis).

In another embodiment the anti-cancer signal transduction inhibitor is an Akt inhibitor (Protein Kinase B) such as API-2, perifosine and RX-0201.

Preferred multitargeted kinase inhibitors include Sutent, (sunitinib, SU-11248), described in U.S. Pat. No. 6,573,293 (Pfizer, Inc, NY, USA) and imatinib mesylate (Gleevec).

Additionally, other targeted anti-cancer agents include the raf inhibitors sorafenib (BAY-43-9006, Bayer/Onyx), GV-1002, ISIS-2503, LE-AON and GI-4000.

The invention also relates to the use of the compounds of the present invention together with cell cycle inhibitors such as the CDK2 inhibitors ABT-751 (Abbott), AZD-5438 (AstraZeneca), Alvocidib (flavopiridol, Aventis), BMS-387,032 (SNS 032 Bristol Myers), EM-1421 (Erimos), indisulam (Esai), seliciclib (Cyclacel), BIO 112 (Onc Bio) UCN-01 (Kyowa Hakko), and AT7519 (Astex Therapeutics) and Pfizer's multitargeted CDK inhibitors PD0332991 and AG24322.

The invention also relates to the use of the compounds of the present invention together with telomerase inhibitors such as transgenic B lymphocyte immunotherapy (Cosmo Bioscience), GRN 163L (Geron), GV1001 (Pharmexa), RO 254020 (and derivatives thereof), and diazaphilonic acid.

Biological response modifiers (such as antibodies, immunotherapeutics and peptide mimics), are agents that modulate defense mechanisms of living organisms or biological responses, such as survival, growth, or differentiation of tissue cells to direct them to have anti-tumor activity.

Immunologicals including interferons and numerous other immune enhancing agents that may be used in combination therapy with 1-[5-(4-amino-7-isopropyl-7H-pyrrolo[2,3-d]pyrimidine-5-carbonyl)-2-methoxy-phenyl]-3-(2,4-dichloro-phenyl)-urea, optionally with one or more other agent include, but are not limited to interferon alpha, interferon alpha-2a, interferon, alpha-2b, interferon beta, interferon gamma-1a, interferon gamma-1b (Actimmune), or interferon gamma-n1, PEG Intron A, and combinations thereof. Other agents include interleukin 2 agonists (such as aldesleukin, BAY-50-4798, Ceplene (histamine dihydrochloride), EMO-273063, MVA-HPV-IL2, HVA-Muc-1-IL2, interleukin 2, teceleukin and Virulizin), Ampligen, Cavaxin, CeaVac (CEA), denileukin, filgrastim, Gastrimmune (C17DT), gemtuzumab ozogamicin, Glutoxim (BAM-002), GMK vaccine (Progenics), Hsp 90 inhibitors (such as HspE7 from Stressgen, AG-858, KOS-953, MV-1-1 and STA-4783), imiquimod, krestin (polysaccharide K), lentinan, Melacine (Corixa), MelVax (mitumomab), molgramostim, Oncophage (HSPPC-96), OncoVAX (including OncoVAX-CL and OncoVAX-Pr), oregovomab, sargramostim, sizofiran, tasonermin, TheraCys, thymalfasin, pemtumomab (Y-muHMFG1), picibanil, Provenge (Dendreon), ubenimex, WF-10 (Immunokine), Z-100 (Ancer-20 from Zeria), Lenalidomide (REVIMID, Celegene), thalomid (Thalidomide), and combinations thereof.

Anti-cancer agents capable of enhancing antitumor immune responses, such as CTLA4 (cytotoxic lymphocyte antigen 4) antibodies, and other agents capable of blocking CTLA4 may also be utilized, such as MDX-010 (Medarex) and CTLA4 compounds disclosed in U.S. Pat. No. 6,682,736. Additional, specific CTLA4 antibodies that can be used in the present invention include those described in U.S. Provisional Application 60/113,647 (filed Dec. 23, 1998), U.S. Pat. No. 6,682,736 both of which are herein incorporated by reference in their entirety.

In another embodiment of the present invention the anti-cancer agent used in conjunction with 1-[5-(4-amino-7-isopropyl-7H-pyrrolo[2,3-d]pyrimidine-5-carbonyl)-2-methoxy-phenyl]-3-(2,4-dichloro-phenyl)-urea and pharmaceutical compositions described herein is a CD20 antagonist. Specific CD20 antibody antagonists that can be used in the present invention include rituximab (Rituxan), Zevalin (Ibritumomab tiuxetan), Bexxar (131-I-tositumomab), Belimumab (LymphoStat-B), HuMax-CD20 (HuMax, Genmab), R 1594 (Roche Genentech), TRU-015 (Trubion Pharmaceuticals) and Ocrelizumab (PRO 70769).

In another embodiment of the present invention the anti-cancer agent used in conjunction with 1-[5-(4-amino-7-isopropyl-7H-pyrrolo[2,3-d]pyrimidine-5-carbonyl)-2-methoxy-phenyl]-3-(2,4-dichloro-phenyl)-urea and pharmaceutical compositions described herein is a CD40 antagonist. Specific CD40 antibody antagonists that can be used in the present invention include CP-870893, CE-35593 and those described in International Patent Application No. WO 2003/040170 which is herein incorporated by reference in its entirety. Other CD40 antagonists include ISF-154 (Ad-CD154, Tragen), toralizumab, CHIR 12.12 (Chiron), SGN 40 (Seattle Genetics) and ABI-793 (Novartis).

In another embodiment of the present invention the anti-cancer agent used in conjunction with 1-[5-(4-amino-7-isopropyl-7H-pyrrolo[2,3-d]pyrimidine-5-carbonyl)-2-methoxy-phenyl]-3-(1,4-dichloro-phenyl)-urea and pharmaceutical compositions described herein is a hepatocyte growth factor receptor antagonist (HGFr or c-MET).

Immunosuppressant agents useful in combination with 1-[5-(4-amino-7-isopropyl-7H-pyrrolo[2,3-d]pyrimidine-5-carbonyl)-2-methoxy-phenyl]-3-(2,4-dichloro-phenyl)-urea include epratuzumab, alemtuzumab, daclizumab, lenograstim and pentostatin (Nipent of Coforin).

The invention also relates to the use of 1-[5-(4-amino-7-isopropyl-7H-pyrrolo[2,3-d]pyrimidine-5-carbonyl)-2-methoxy-phenyl]-3-(2,4-dichloro-phenyl)-urea together with hormonal, anti-hormonal, anti-androgenal therapeutic agents such as anti-estrogens including, but not limited to fulvestrant, toremifene, raloxifene, lasofoxifene, letrozole (Femara, Novartis), anti-androgens such as bicalutamide, finasteride, flutamide, mifepristone, nilutamide, Casodex® (4′-cyano-3-(4-fluorophenylsulphonyl)-2-hydroxy-2-methyl-3′-(trifluoromethyl)-propionanilide, biocalutamide) and combinations thereof.

The invention also contemplates the use of the compounds of the present invention together with hormonal therapy, including but not limited to, exemestane (Aromasin, Pfizer Inc.), Abarelix (Praecis), Trelstar, anastrozole (Arimidex, Astrazeneca), Atamestane (Biomed-777), Atrasentan (Xinlay), Bosentan, Casodex (AstraZeneca), doxercalciferol, fadrozole, formestane, gosrelin (Zoladex, AstraZeneca), Histrelin (histrelin acetate), letrozole. leuprorelin (Lupron or Leuplin, TAP/Abbott/Takeda), tamoxifen citrate (tamoxifen, Nolvadex, AstraZeneca), and combinations thereof.

The invention also contemplates the use of the compounds of the present invention together with gene silencing agents or gene activating agents such as histone deacetylase (HDAC) inhibitors such as suberolanilide hydroxamic acid (SAHA, Merck Inc./Aton Pharmaceuticals), depsipeptide (FR901228 or FK228), G2M-777 MS-275, pivaloyloxymethyl butyrate and PXD-101.

The invention also contemplates the use of the compounds of the present invention together with gene therapeutic agents such as Advexin (ING 201), TNFerade (GeneVec, a compound which express TNFalpha in response to radiotherapy), RB94 (Baylor College of Medicine).

The invention also contemplates the use of the compounds of the present invention together with ribonucleases such as Onconase (ranpimase).

The invention also contemplates the use of the compounds of the present invention together with antisense oligonucleotides such as bcl-2 antisense inhibitor Genasense (Oblimersen, Genta.

The invention also contemplates the use of the compounds of the present invention together with proteosomics such as PS-341 (MLN-341) and Velcade (bortezomib).

The invention also contemplates the use of the compounds of the present invention together with anti-vascular agents such as Combretastatin A4P (Oxigene).

The invention also contemplates the use of the compounds of the present invention together with traditional cytotoxic agents including DNA binding agents, mitotic inhibitors, alkylating agents, anti-metabolites, intercalating antibiotics, topoisomerase inhibitors and microtubulin inhibitors.

Topoisomerase I inhibitors useful in the combination embodiments of the present invention include 9-aminocamptothecin, belotecan, BN-80915 (Roche), camptothecin, diflomotecan, edotecarin, exatecan (Daiichi), gimatecan, 10-hydroxycamptothecin, irinotecan HOI (Camptosar), lurtotecan, Orathecin (rubitecan, Supergen), SN-38, topotecan, and combinations thereof.

Camptothecin derivatives are of particular interest in the combination embodiments of the invention and include camptothecin, 10-hydroxycamptothecin, 9-aminocamptothecin, irinotecan, SN-38, edotecarin, topotecan and combinations thereof.

A particularly preferred toposimerase I inhibitor is irinotecan HCl (Camptosar).

Topoisomerase II inhibitors useful in the combination embodiments of the present invention include aclarubicin, adriamycin, amonafide, amrubicin, annamycin, daunorubicin, doxorubicin, elsamitrucin, epirubicin, etoposide, idarubicin, galarubicin, hydroxycarbamide, nemorubicin, novantrone (mitoxantrone), pirarubicin, pixantrone, procarbazine, rebeccamycin, sobuzoxane, tafluposide, valrubicin, and Zinecard (dexrazoxane).

Particularly preferred toposimerase II inhibitors include epirubicin (Ellence), doxorubicin, dauntorubicin, idarubicin and etoposide.

Alkylating agents that may be used in combination therapy with 1-[5-(4-amino-7-isopropyl-7H-pyrrolo[2,3-d]pyrimidine-5-carbonyl)-2-methoxy-phenyl]-3-(2,4-dichloro-phenyl)-urea, optionally with one or more other agents include, but are not limited to, nitrogen mustard N-oxide, cyclophosphamide, AMD-473, altretamine, AP-5280, apaziquone, brostallicin, bendamustine, busulfan, carboquone, carmustine, chlorambucil, dacarbazine, estramustine, fotemustine, glufosfamide, ifosfamide, KW-2170, lomustine, mafosfamide, mechlorethamine, melphalan, mitobronitol, mitolactol, mitomycin C, mitoxatrone, nimustine, ranimustine, temozolomide, thiotepa, and platinum-coordinated alkylating compounds such as cisplatin, Paraplatin (carboplatin), eptaplatin, lobaplatin, nedaplatin, Eloxatin (oxaliplatin, Sanofi), streptozocin, or satrplatin and combinations thereof.

Particularly preferred alkylating agents include Eloxatin (oxaliplatin).

Antimetabolites that may be used in combination therapy with 1-[5-(4-amino-7-isopropyl-7H-pyrrolo[2,3-d]pyrimidine-5-carbonyl)-2-methoxy-phenyl]-3-(2,4-dichloro-phenyl)-urea, optionally with one or more other agents include, but are not limited to dihydrofolate reductase inhibitors (such as methotrexate and NeuTrexin (trimetresate glucuronate)), purine antagonists (such as 6-mercaptopurine riboside, mercaptopurine, 6-thioguanine, cladribine, clofarabine (Clolar), fludarabine, nelarabine, and raltitrexed), pyrimidine antagonists (such as 5-fluorouracil (5-FU), Alimta (premetrexed disodium, LY231514, MTA), capecitabine (Xeloda), cytosine arabinoside, Gemzar (gemcitabine, Eli Lilly), Tegafur (UFT Orzel or Uforal and including TS-1 combination of tegafur, gimestat and otostat), doxifluridine, carmofur, cytarabine (including octosfate, phosphate stearate, sustained release and liposomal forms), enocitabine, 5-azacitidine (Vidaza), decitabine, and ethynylcytidine) and other antimetabolites such as eflomithine, hydroxyurea, leucovorin, nolatrexed (Thymitaq), triapine, trimetrexate, or for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid and combinations thereof.

In another embodiment the anti-cancer agent is a poly(ADP-ribose) polymerase-1 (PARP-1) inhibitor such as AG-014699, ABT-472 INO-1001, KU-0687 and GPI 81610.

Microtubulin inhibitors that may be used in combination therapy with 1-[5-(4-amino-7-isopropyl-7H-pyrrolo[2,3-d]pyrimidine-5-carbonyl)-2-methoxy-phenyl]-3-(2,4-dichloro-phenyl)-urea, optionally with one or more other agents include, but are not limited to ABI-007, Albendazole, Batabulin, CPH-82, EPO 906 (Novartis), discodermolide (XAA-296), Vinfunine and ZD-6126 (AstraZeneca).

Antibiotics that may be used in combination therapy with 1-[5-(4-amino-7-isopropyl-7H-pyrrolo[2,3-d]pyrimidine-5-carbonyl)-2-methoxy-phenyl]-3-(2,4-dichloro-phenyl)-urea optionally with one or more other agent including, but are not limited to, intercalating antibiotics such as actinomycin D, bleomycin, mitomycin C, neocarzinostatin (Zinostatin), peplomycin, and combinations thereof.

Plant derived anti-tumor substances (also known as spindle inhibitors) that may be used in combination therapy with 1-[5-(4-amino-7-isopropyl-7H-pyrrolo[2,3-d]pyrimidine-5-carbonyl)-2-methoxy-phenyl]-3-(2,4-dichloro-phenyl)-urea, optionally with one or more other agent include, but are not limited to, mitotic inhibitors, for example vinblastine, vincristine, vindesine, vinorelbine (Navelbine), docetaxel (Taxotere), Ortataxel, paclitaxel (including Taxoprexin a DHA/paciltaxel conjugate) and combinations thereof.

Platinum-coordinated compounds include but are not limited to, cisplatin, carboplatin, nedaplatin, oxaliplatin (Eloxatin), Satraplatin (JM-216), and combinations thereof.

Particularly preferred cytotoxic agents include Camptosar, capecitabine (Xeloda), oxaliplatin (Eloxatin), Taxotere and combinations thereof.

Other antitumor agents include alitretinoin I-asparaginase, AVE-8062 (Aventis), calcitriol (Vitamin D derivative), Canfosfamide (Telcyta, TLK-286), Cotara (131I chTNT 1/b), DMXAA (Antisoma), exisulind, ibandronic acid, Miltefosine, NBI-3001 (IL-4), pegaspargase, RSR13 (efaproxiral), Targretin (bexarotene), tazarotne (Vitamin A derivative), Tesmilifene (DPPE), Theratope, tretinoin, Trizaone (tirapazamine), Xcytrin (motexafin gadolinium) and Xyotax (polyglutamate paclitaxel), and combinations thereof.

In another embodiment of the present invention statins may be used in conjunction with 1-[5-(4-amino-7-isopropyl-7H-pyrrolo[2,3-d]pyrimidine-5-carbonyl)-2-methoxy-phenyl]-3-(2,4-dichloro-phenyl)-urea. Statins (HMG-CoA reducatase inhibitors) may be selected from the group consisting of Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin and Niacin (Advicor, Kos Pharmaceuticals), derivatives and combinations thereof.

In a preferred embodiment the statin is selected from the group consisting of Atovorstatin and Lovastatin, derivatives and combinations thereof.

Other agents useful as anti-tumor agents include Caduet, Lipitor and torcerapib.

Another embodiment of the present invention of particular interest relates to a method for the treatment of breast cancer in a human in need of such treatment, comprising administering to said human an amount of 1-[5-(4-amino-7-isopropyl-7H-pyrrolo[2,3-d]pyrimidine-5-carbonyl)-2-methoxy-phenyl]-3-(2,4-dichloro-phenyl)-urea (including hydrates, solvates and polymorphs or pharmaceutically acceptable salts thereof), in combination with one or more (preferably one to three) anti-cancer agents selected from the group consisting of trastuzumab (Herceptin), docetaxel (Taxotere), paclitaxel, capecitabine (Xeloda), gemcitabine (Gemzar), vinorelbine (Navelbine), exemestane (Aromasin), letrozole (Femara) and anastrozole (Arimidex).

Another embodiment of the present invention of particular interest relates to a method for the treatment of colorectal cancer in a human in need of such treatment, comprising administering to said human an amount of 1-[5-(4-amino-7-isopropyl-7H-pyrrolo[2,3-d]pyrimidine-5-carbonyl)-2-methoxy-phenyl]-3-(2,4-dichloro-phenyl)-urea (including hydrates, solvates and polymorphs or pharmaceutically acceptable salts thereof), in combination with one or more (preferably one to three) anti-cancer agents selected from the group consisting of capecitabine (Xeloda), irinotecan HCl (Camptosar), bevacizumab (Avastin), cetuximab (Erbitux), oxaliplatin (Eloxatin), premetrexed disodium (Alimta), vatalanib (PTK-787), Sutent (sunitinib), AG-13736 (axitinib), SU-14843, PF-0337210, PD-32590, PF-2341066, Tarceva, Iressa, Pelitinib, Lapatinib, Mapatumtumab, Gleevec, BMS 184476, CCI 779, ISIS 2503, ONYX 015 and Flavopyridol, wherein the amounts of the active agent together with the amounts of the combination anticancer agents is effective in treating colorectal cancer.

Another embodiment of the present invention of particular interest relates to a method for the treatment of renal cell carcinoma in a human in need of such treatment, comprising administering to said human an amount of 1-[5-(4-amino-7-isopropyl-7H-pyrrolo[2,3-d]pyrimidine-5-carbonyl)-2-methoxy-phenyl]-3-(2,4-dichloro-phenyl)-urea (including hydrates, solvates and polymorphs or pharmaceutically acceptable salts thereof), in combination with one or more (preferably one to three) anti-cancer agents selected from the group consisting of capecitabine (Xeloda), interferon alpha, interleukin-2, bevacizumab (Avastin), gemcitabine (Gemzar), thalidomide, cetuximab (Erbitux), vatalanib (PTK-787), Sutent, AG-13736, SU-11248, Tarceva, Iressa, Lapatinib and Gleevec, wherein the amounts of the active agent together with the amounts of the combination anticancer agents is effective in treating renal cell carcinoma.

Another embodiment of the present invention of particular interest relates to a method for the treatment of melanoma in a human in need of such treatment, comprising administering to said human an amount of 1-[5-(4-amino-7-isopropyl-7H-pyrrolo[2,3-d]pyrimidine-5-carbonyl)-2-methoxy-phenyl]-3-(2,4-dichloro-phenyl)-urea (including hydrates, solvates and polymorphs or pharmaceutically acceptable salts thereof), in combination with one or more (preferably one to three) anti-cancer agents selected from the group consisting of interferon alpha, interleukin-2, temozolomide, docetaxel (Taxotere), paclitaxel, DTIC, PD-325,901, Axitinib, bevacizumab (Avastin), thalidomide, sorafanib, vatalanib (PTK-787), Sutent, CpG-7909, AG-13736, Iressa, Lapatinib and Gleevec, wherein the amounts of the active agent together with the amounts of the combination anticancer agents is effective in treating melanoma.

Another embodiment of the present invention of particular interest relates to a method for the treatment of Lung cancer in a human in need of such treatment, comprising administering to said human an amount of 1-[5-(4-amino-7-isopropyl-7H-pyrrolo[2,3-d]pyrimidine-5-carbonyl)-2-methoxy-phenyl]-3-(2,4-dichloro-phenyl)-urea (including hydrates, solvates and polymorphs or pharmaceutically acceptable salts thereof, in combination with one or more (preferably one to three) anti-cancer agents selected from the group consisting of capecitabine (Xeloda), bevacizumab (Avastin), gemcitabine (Gemzar), docetaxel (Taxotere), paclitaxel, premetrexed disodium (Alimta), Tarceva, Iressa, and Paraplatin (carboplatin), wherein the amounts of the active agent together with the amounts of the combination anticancer agents is effective in treating Lung cancer.

In one preferred embodiment radiation can be used in conjunction with 1-[5-(4-amino-7-isopropyl-7H-pyrrolo[2,3-d]pyrimidine-5-carbonyl)-2-methoxy-phenyl]-3-(2,4-dichloro-phenyl)-urea and pharmaceutical compositions described herein. Radiation may be administered in a variety of fashions. For example, radiation may be electromagnetic or particulate in nature. Electromagnetic radiation useful in the practice of this invention includes, but is not limited, to x-rays and gamma rays. In a preferable embodiment, supervoltage x-rays (x-rays>=4 MeV) may be used in the practice of this invention. Particulate radiation useful in the practice of this invention includes, but is not limited to, electron beams, protons beams, neutron beams, alpha particles, and negative pi mesons. The radiation may be delivered using conventional radiological treatment apparatus and methods, and by intraoperative and stereotactic methods. Additional discussion regarding radiation treatments suitable for use in the practice of this invention may be found throughout Steven A. Leibel et al., Textbook of Radiation Oncology (1998) (publ. W. B. Saunders Company), and particularly in Chapters 13 and 14. Radiation may also be delivered by other methods such as targeted delivery, for example by radioactive “seeds,” or by systemic delivery of targeted radioactive conjugates, J. Padawer et al., Combined Treatment with Radioestradiol Iucanthone in Mouse C3HBA Mammary Adenocarcinoma and with Estradiol Iucanthone in an Estrogen Bioassay, Int. J. Radiat. Oncol. Biol. Phys. 7:347-357 (1981). Other radiation delivery methods may be used in the practice of this invention.

The amount of radiation delivered to the desired treatment volume may be variable. In a preferable embodiment, radiation may be administered in amount effective to cause the arrest or regression of the cancer, in combination with 1-[5-(4-amino-7-isopropyl-7H-pyrrolo[2,3-d]pyrimidine-5-carbonyl)-2-methoxy-phenyl]-3-(2,4-dichloro-phenyl)-urea (including all forms and formulations thereof and pharmaceutical compositions thereof.

In a more preferable embodiment, radiation is administered in at least about 1 Gray (Gy) fractions at least once every other day to a treatment volume, still more preferably radiation is administered in at least about 2 Gray (Gy) fractions at least once per day to a treatment volume, even more preferably radiation is administered in at least about 2 Gray (Gy) fractions at least once per day to a treatment volume for five consecutive days per week.

In a more preferable embodiment, radiation is administered in 3 Gy fractions every other day, three times per week, to a treatment volume.

In yet another more preferable embodiment, a total of at least about 20 Gy, still more preferably at least about 30 Gy, most preferably at least about 60 Gy of radiation is administered to a host in need thereof.

In one more preferred embodiment of the present invention 14 GY radiation is administered.

In another more preferred embodiment of the present invention 10 GY radiation is administered.

In another more preferred embodiment of the present invention 7 GY radiation is administered.

In a most preferable embodiment, radiation is administered to the whole brain of a host, wherein the host is being treated for metastatic cancer.

Further, the invention provides a compound of the present invention alone or in combination with one or more supportive care products, e.g., a product selected from the group consisting of Filgrastim (Neupogen), ondansetron (Zofran), Fragmin, Procrit, Aloxi, Emend, or combinations thereof.

### FIGURES

FIG. 1: Phosphate salt Form A x-ray powder diffraction pattern.

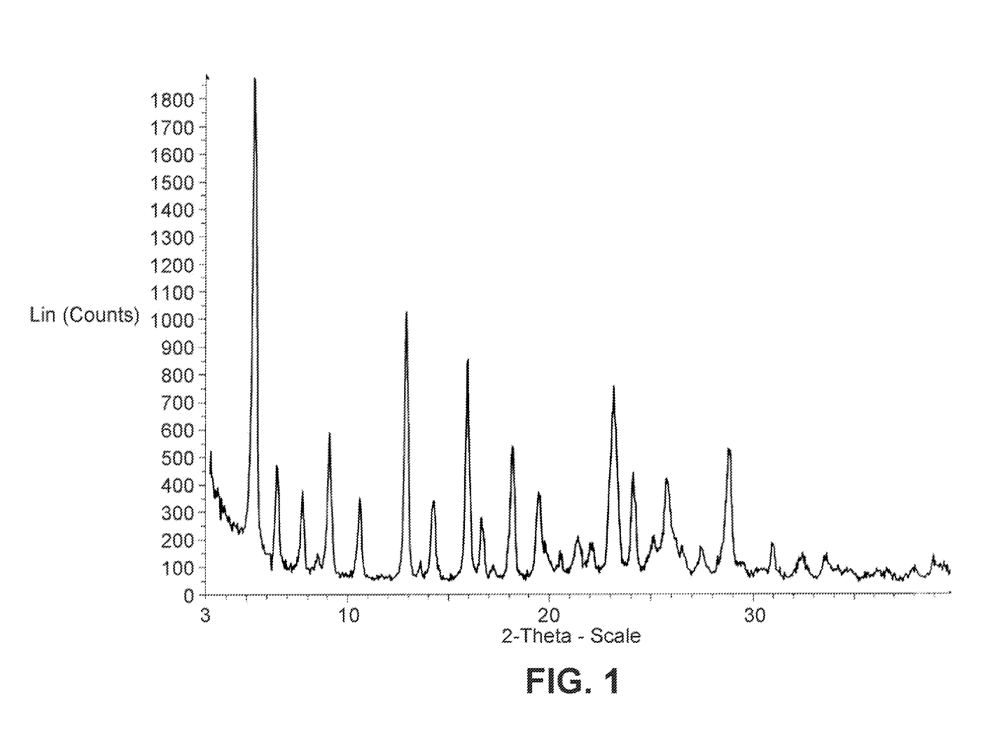

FIG. 2: Phosphate salt Form A differential scanning calorimeter plot.

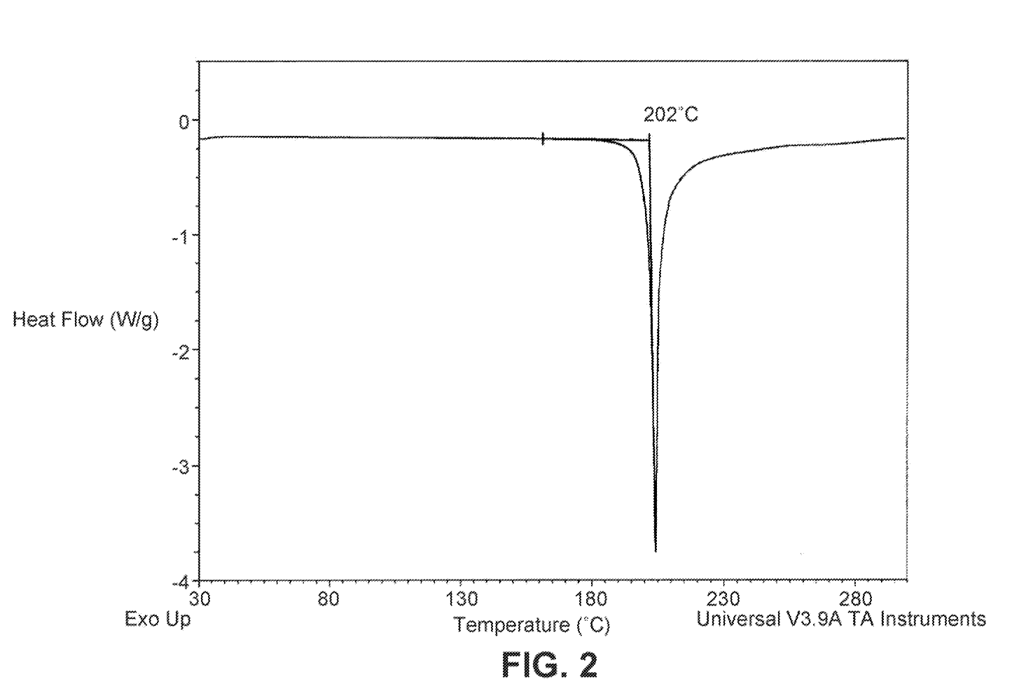

FIG. 3: Phosphate salt Form B x-ray powder diffraction pattern.

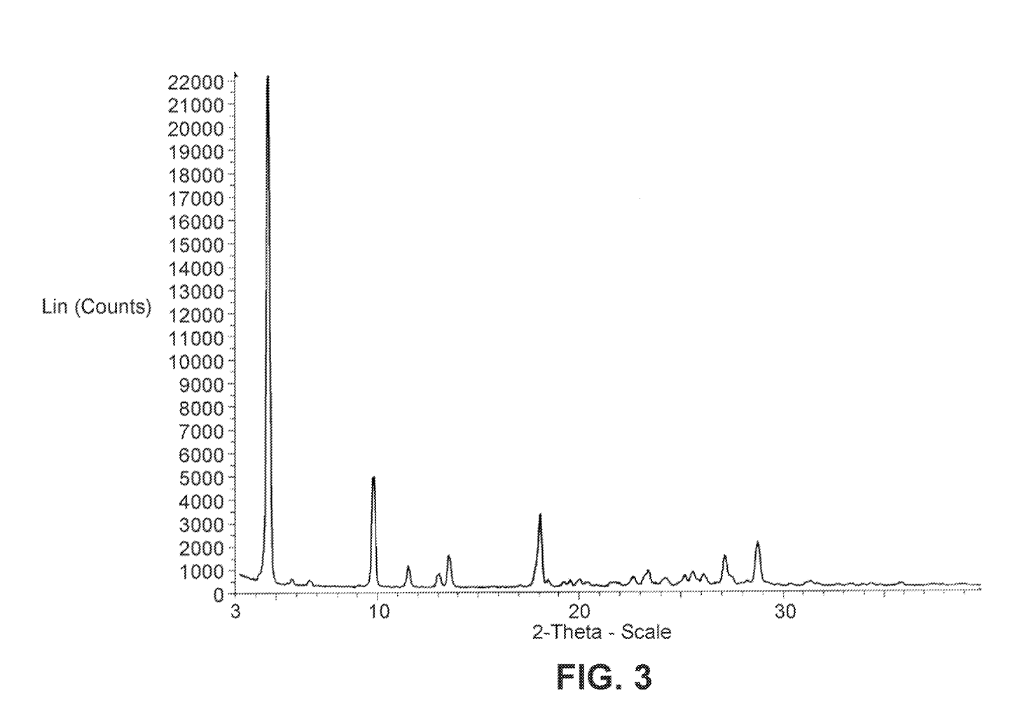

FIG. 4: Mesylate salt Form A x-ray powder diffraction pattern.

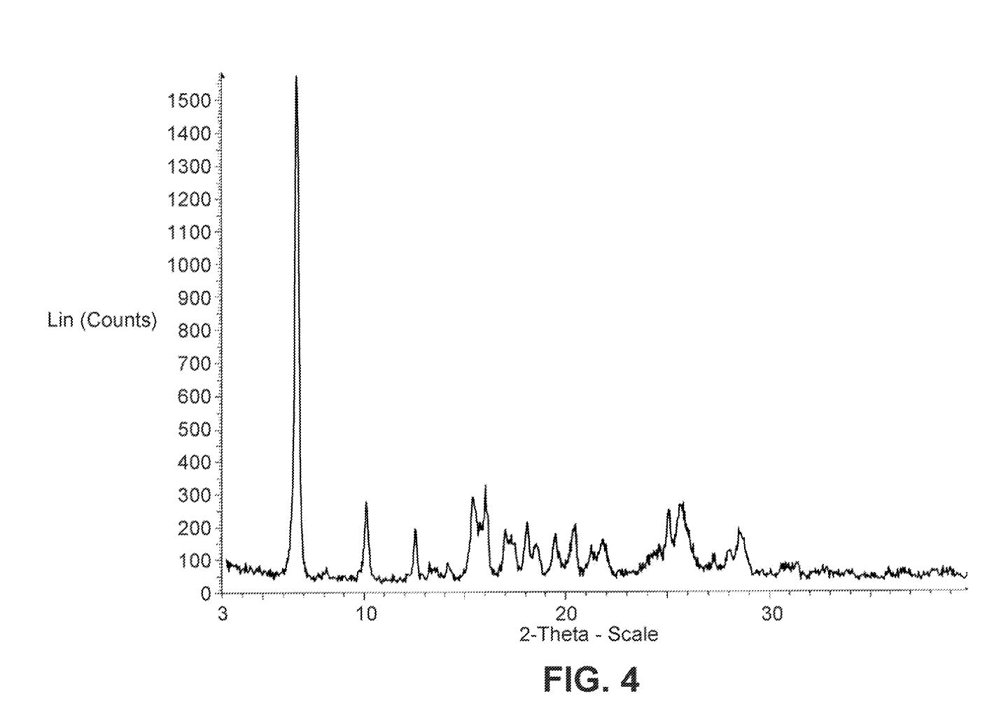

FIG. 5: Mesylate salt Form A differential scanning calorimeter plot.

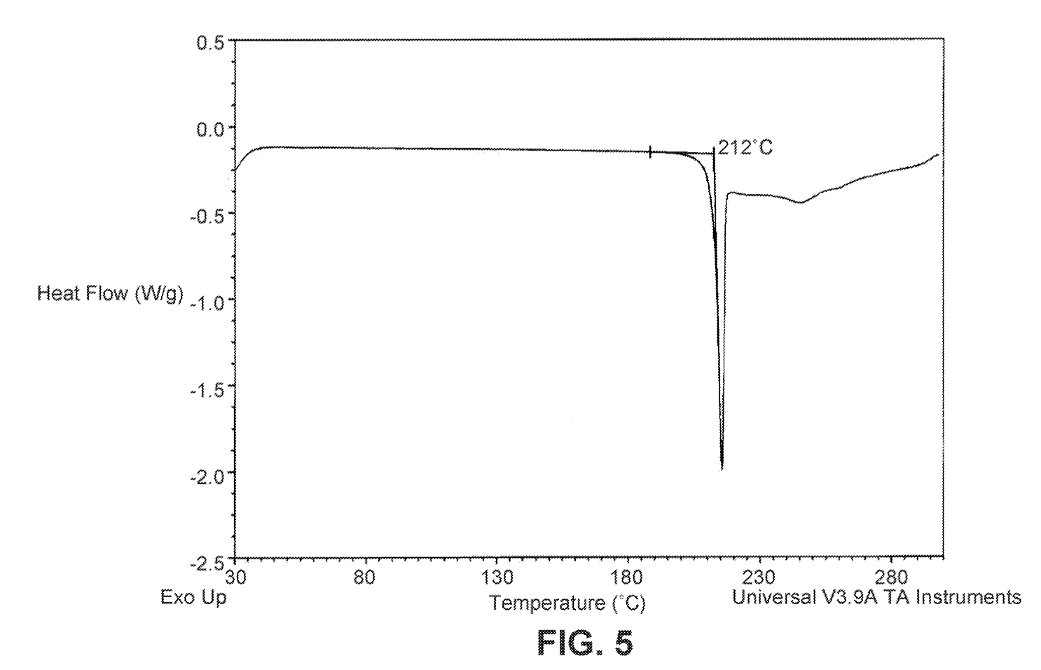

FIG. 6: Mesylate salt Form B x-ray powder diffraction pattern.

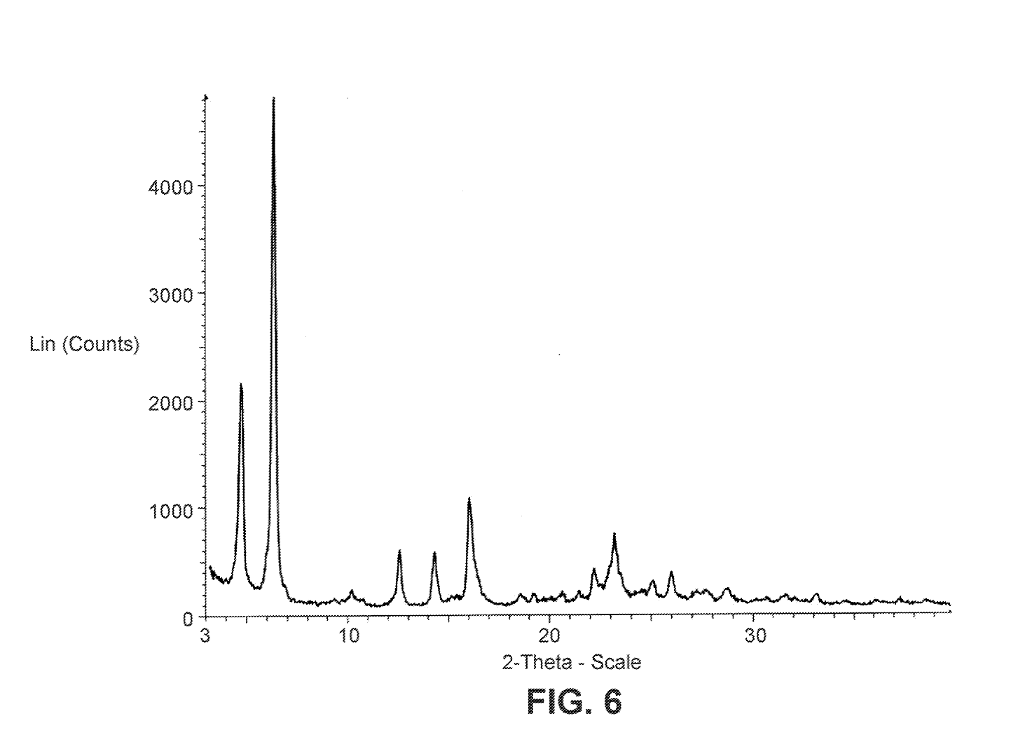

FIG. 7: Mesylate salt Form B differential scanning calorimeter plot.

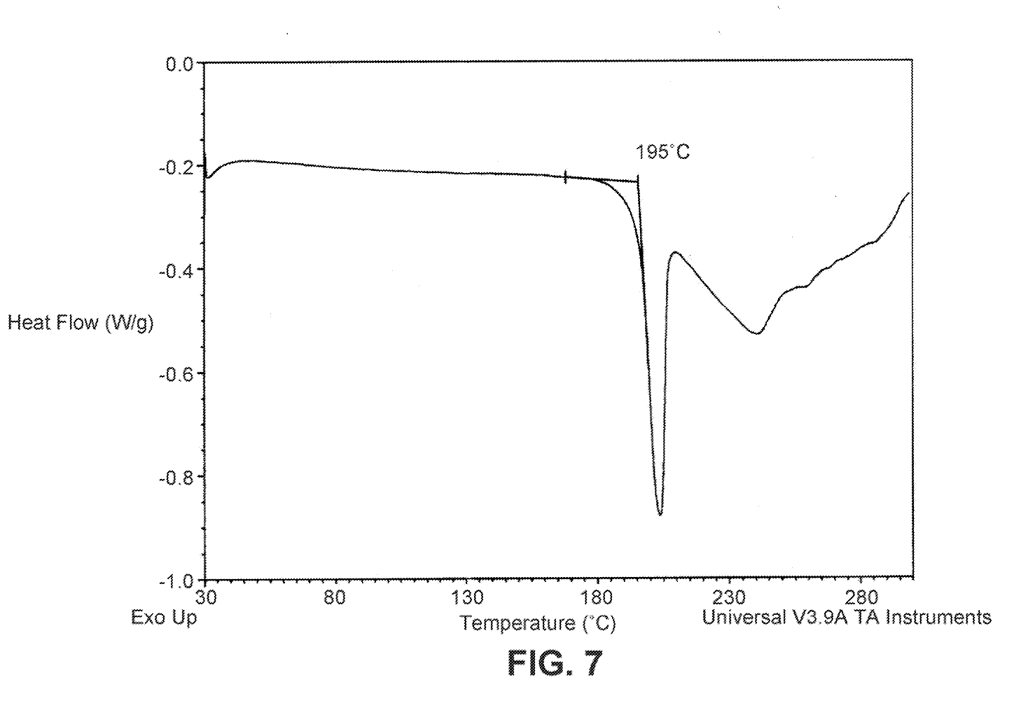

FIG. 8: Mesylate salt Form C x-ray powder diffraction pattern.

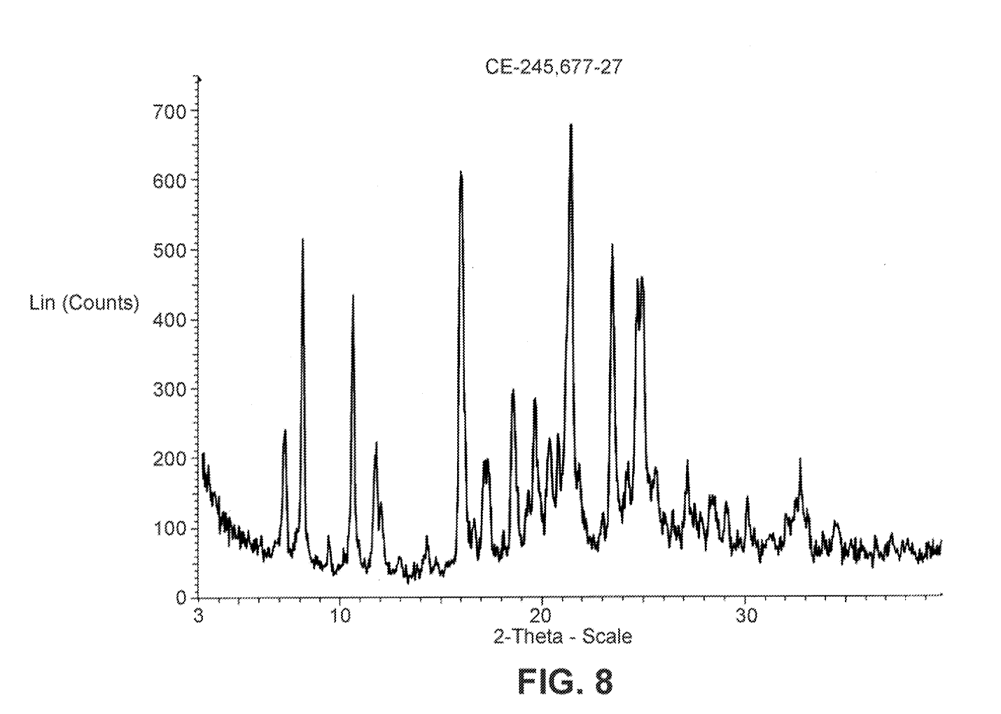

FIG. 9: Mesylate salt Form C differential scanning calorimeter plot.

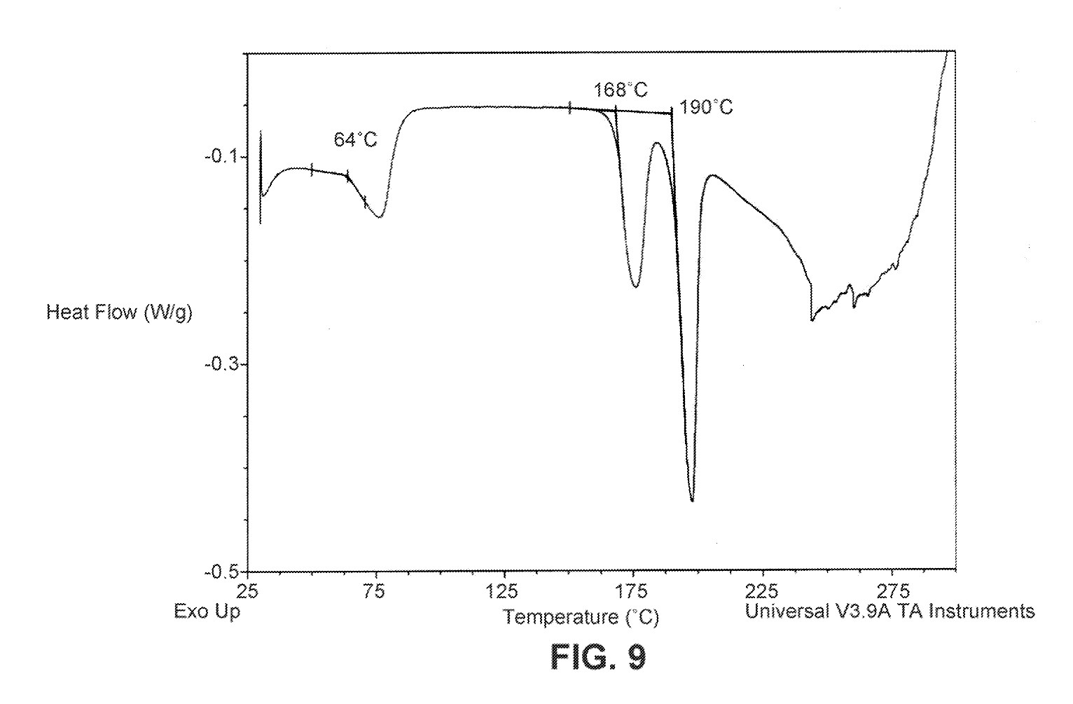

FIG. 10: Besylate salt Form A x-ray powder diffraction pattern.

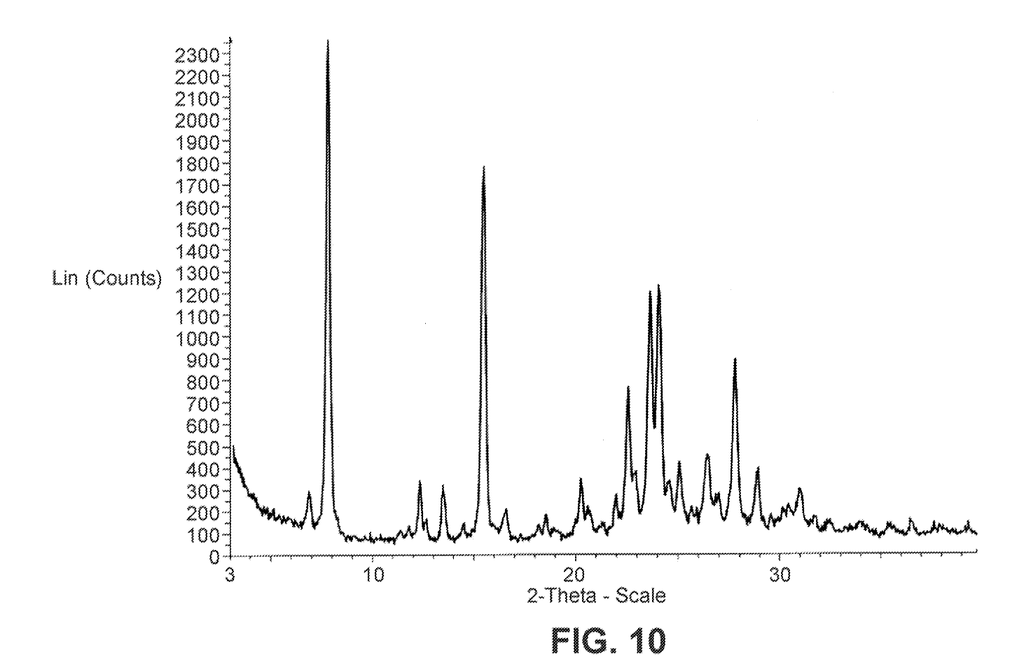

FIG. 11: Besylate salt Form A differential scanning calorimeter plot.

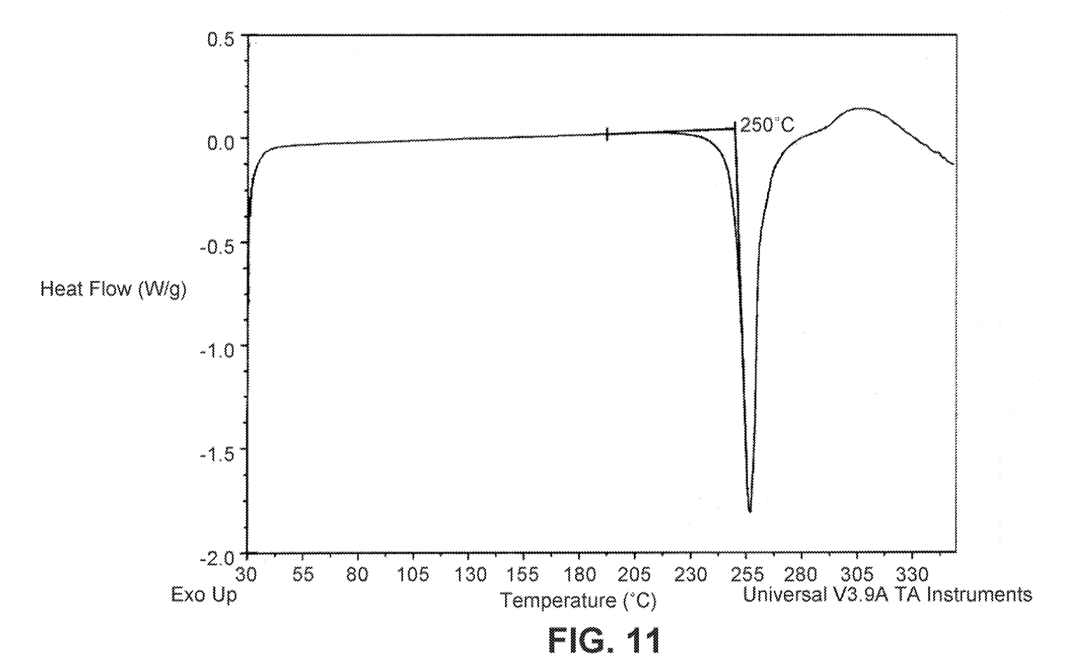

FIG. 12: Tosylate salt Form A x-ray powder diffraction pattern.

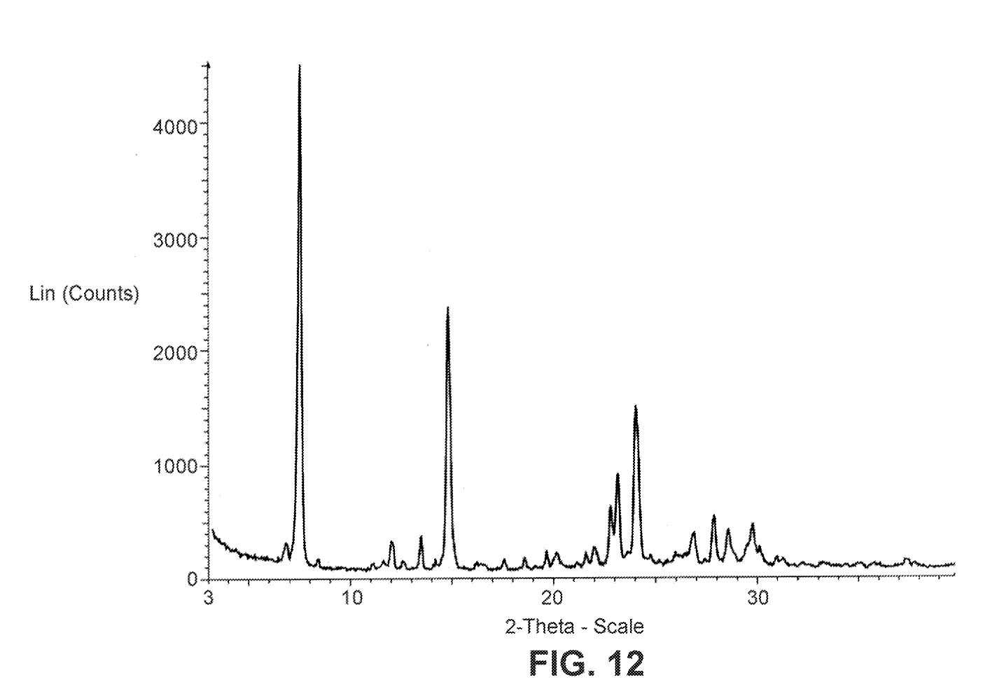

FIG. 13: Tosylate salt Form A differential scanning calorimeter plot.

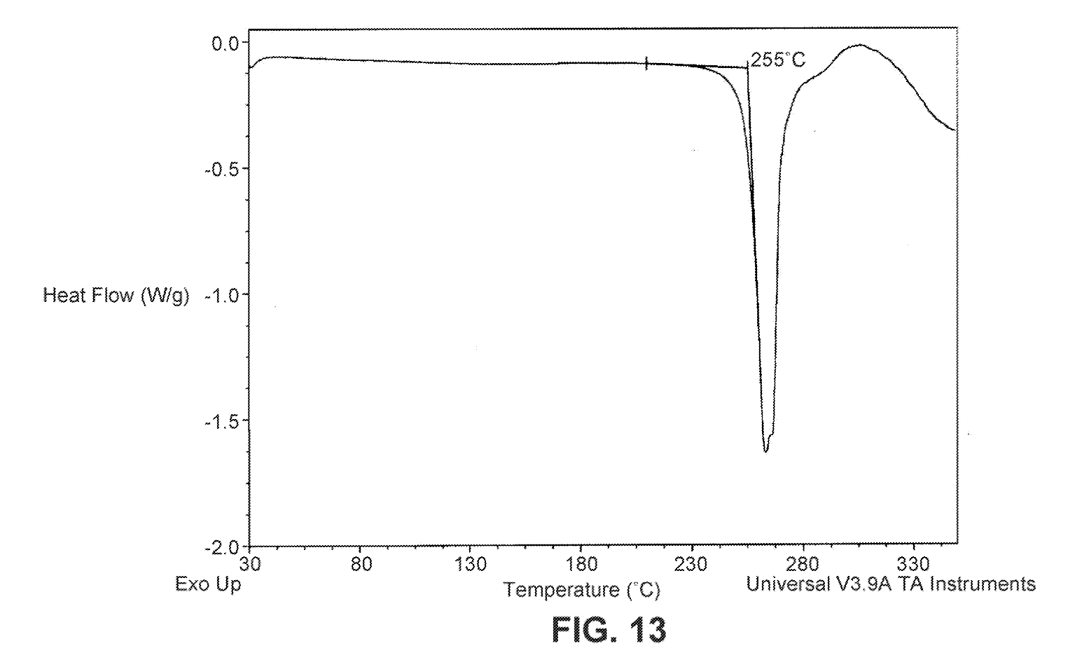

FIG. 14: Hydrochloride salt Form A x-ray powder diffraction pattern.

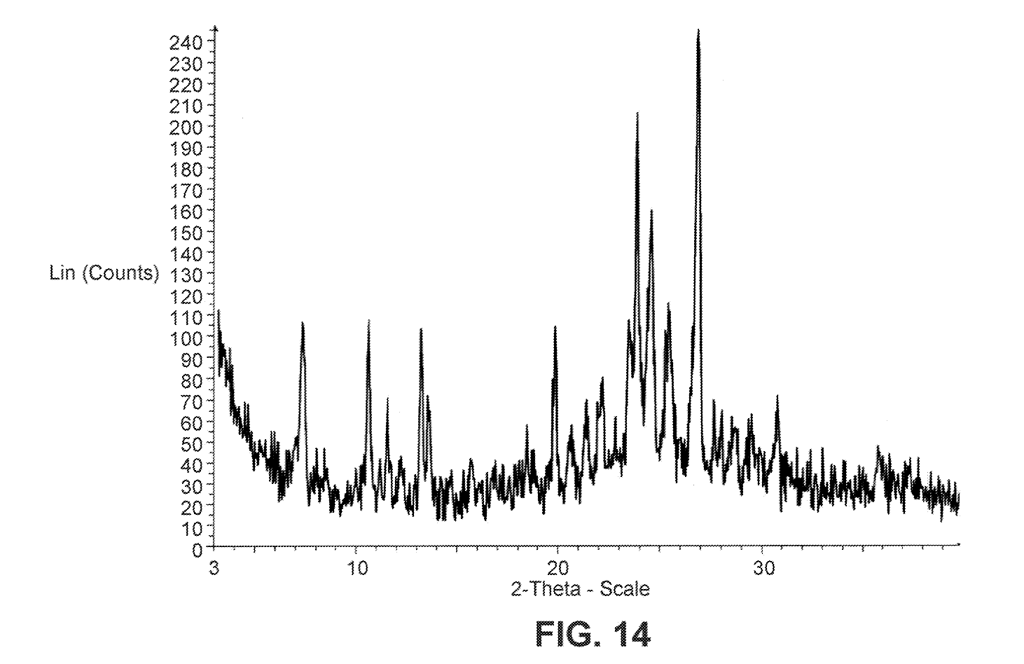

### DETAILED DESCRIPTION OF THE INVENTION

### 1-[5-(4-Amino-7-isopropyl-7H-pyrrolo[2,3-d]pyrimidine-5carbonyl)-2-methoxy-phenyl]-3-(2,4-dichloro-phenyl)-urea

1-[5-(4-Amino-7-isopropyl-7H-pyrrolo[2,3-d]pyrimidine-5-carbonyl)-2-methoxy-phenyl]-3-(2,4-dichloro-phenyl)-urea can be made according to the methods described in International Patent Publication WO 04/056830, published Jul. 8, 2004. Alternatively, 1-[5-(4-amino-7-isopropyl-7H-pyrrolo[2,3-d]pyrimidine-5-carbonyl)-2-methoxy-phenyl]-3-(2,4-dichloro-phenyl)-urea can be made according to the examples described below.

As stated above, the present invention relates to novel crystalline and non-crystalline forms and formulations of 1-[5-(4-amino-7-isopropyl-7H-pyrrolo[2,3-d]pyrimidine-5-carbonyl)-2-methoxy-phenyl]-3-(2,4-dichloro-phenyl)-urea.

One form of the active agent of the present invention includes salts of 1-[5-(4-amino-7-isopropyl-7H-pyrrolo[2,3-d]pyrimidine-5-carbonyl)-2-methoxy-phenyl]-3-(2,4-dichloro-phenyl)-urea. 1-[5-(4-Amino-7-isopropyl-7H-pyrrolo[2,3-d]pyrimidine-5-carbonyl)-2-methoxy-phenyl]-3-(2,4-dichloro-phenyl)-urea is basic in nature and is thus capable of forming a wide variety of different salts with various inorganic and organic acids. Although such salts must be pharmaceutically acceptable for administration to mammals preferably humans, it is often desirable in practice to initially isolate 1-[5-(4-amino-7-isopropyl-7H-pyrrolo[2,3-d]pyrimidine-5-carbonyl)-2-methoxy-phenyl]-3-(2,4-dichloro-phenyl)-urea from the reaction mixture as a pharmaceutically unacceptable salt and then simply convert the later back to the free base compound by treatment with an alkaline reagent and subsequently convert the latter free base to a pharmaceutically acceptable acid addition salt. The acid addition salt of 1-[5-(4-amino-7-isopropyl-7H-pyrrolo[2,3-d]pyrimidine-5-carbonyl)-2-methoxy-phenyl]-3-(2,4-dichloro-phenyl)-urea of this invention is readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid. Specific preparations of salts are described below.

Other forms of 1-[5-(4-amino-7-isopropyl-7H-pyrrolo[2,3-d]pyrimidine-5-carbonyl)-2-methoxy-phenyl]-3-(2,4-dichloro-phenyl)-urea include any acceptable crystalline and non-crystalline solvate, hydrate, isomorph, polymorph, or prodrug of the freebase or aforementioned salts of 1-[5-(4-amino-7-isopropyl-7H-pyrrolo[2,3-d]pyrimidine-5-carbonyl)-2-methoxy-phenyl]-3-(2,4-dichloro-phenyl)-urea.

The most preferred form of 1-[5-(4-amino)-7-isopropyl-7H-pyrrolo[2,3-d]pyrimidine-5-carbonyl)-2-methoxy-phenyl]-3-(2,4-dichloro-phenyl)-urea for formulation in the solid amorphous dispersion, preferably the SDD, is its freebase.

1-[5-(4-Amino-7-isopropyl-7H-pyrrolo[2,3-d]pyrimidine-5-carbonyl)-2-methoxy-phenyl]-3-(2,4-dichloro-phenyl)-urea is an inhibitor/antagonist of various enzymes/receptors. It is active against a variety of kinase targets which are involved in angiogenesis/vasculogenesis, oncogenic and protooncogenic signal transduction and cell cycle regulations. As such, 1-[5-(4-Amino-7-isopropyl-7H-pyrrolo[2,3-d]pyrimidine-5-carbonyl)-2-methoxy-phenyl]-3-(2,4-dichloro-phenyl)-urea is useful in the prevention and treatment of a variety of human hyperproliferative disorders such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, and other hyperplastic conditions such as benign hyperplasia of the prostate (e.g., BPH). It is, in addition, expected that 1-[5-(4-amino-7-isopropyl-7H-pyrrolo[2,3-d]pyrimidine-5-carbonyl)-2-methoxy-phenyl]-3-(2,4-dichloro-phenyl)-urea possesses activity against a range of leukemias and lymphoid malignancies.

1-[5-(4-Amino-7-isopropyl-7H-pyrrolo[2,3-d]pyrimidine-5-carbonyl)-2-methoxy-phenyl]-3-(2,4-dichloro-phenyl)-urea is also useful in the treatment of additional disorders in which aberrant ligand/receptor expression, interaction, activation or signal events related to various protein kinases, are involved. Such disorders include those of neuronal, glial, astrocytal, hypothalamic, and other glandular macrophagal, epithelia, stromal, and blastocoelic naturein which aberrant function, expression, activation or signaling of a protein kinase are involved. In addition, 1-[5-(4-amino-7-isopropyl-7H-pyrrolo[2,3-d]pyrimidine-5-carbonyl)-2-methoxy-phenyl]-3-(2,4-dichloro-phenyl)-urea may have therapeutic utility in inflammatory, angiogenic and immunologic disorders involving both identified and as yet unidentified kinases that are inhibited by 1-[5-(4-amino-7-isopropyl-7H-pyrrolo[2,3-d]pyrimidine-5-carbonyl)-2-methoxy-phenyl]-3-(2,4-dichloro-phenyl)-urea.

The present invention relates to solid amorphous dispersion compositions of 1-[5-(4-amino-7-isopropyl-7H-pyrrolo[2,3-d]pyrimidine-5-carbonyl)-2-methoxy-phenyl]-3-(2,4-dichloro-phenyl)-urea and at least one concentration-enhancing polymer. More specifically, the solid amorphous dispersion compositions of the present invention provide enhancements in aqueous concentration in an environment of use and in bioavailability compared with other conventional compositions. The compositions, active agent, suitable polymers, and optional excipients are discussed in more detail as follows.

Spray Dry Dispersion Compositions of 1-[5-(4-amino-7-isopropyl-7H-pyrrolo[2,3-d]pyrimidine-5-carbonyl)-2-methoxy-phenyl]-3-(2,4-dichloro-phenyl)-urea and Concentration-enhancing Polymers

The solid amorphous dispersion compositions, preferably a spray dry dispersion, of the present invention comprise dispersions of the active agent and at least one concentration-enhancing polymer. The active agent in the dispersion may be crystalline or amorphous. Preferably, at least a major portion of the active agent in the composition is amorphous. By “amorphous” is meant simply that the active agent is in a non-crystalline state. As used herein, the term “a major portion” of the active agent means that at least 60 percent of the active agent in the composition is in the amorphous form, rather than the crystalline form. Preferably, the active agent in the dispersion is substantially amorphous. As used herein, “substantially amorphous” means that the amount of the active agent in the crystalline form does not exceed about 25 percent. More preferably, the active agent, in the dispersion is “almost completely amorphous” meaning that the amount of active agent in the crystalline form does not exceed about 10 percent. Amounts of crystalline 1-[5-(4-amino-7-isopropyl-7H-pyrrolo[2,3-d]pyrimidine-5-carbonyl)-2-methoxy-phenyl]-3-(2,4-dichloro-phenyl)-urea may be measured by powder X-ray diffraction, Scanning Electron Microscope (SEM) analysis, differential scanning calorimetry (DSC), or any other standard quantitative measurement.

The SDD composition may contain from about 1 to about 80 weight percent 1-[5-(4-amino-7-isopropyl-7H-pyrrolo[2,3-d]pyrimidine-5-carbonyl)-2-methoxy-phenyl]-3-(2,4-dichloro-phenyl)-urea, depending an the dose of active agent and the effectiveness of the concentration-enhancing polymer. Enhancement of aqueous 1-[5-(4-amino-7-isopropyl-7H-pyrrolo[2,3-d]pyrimidine-5-carbonyl)-2-methoxy-phenyl]-3-(2,4-dichloro-phenyl)-urea concentrations and relative bioavailability are typically best at low inhibitor levels, typically less than about 25 to 40 wt percent. However, due to the practical limit of the dosage form size, higher inhibitor levels are often preferred and in many cases perform well.

The amorphous inhibitor can exist within the solid amorphous dispersion as a pure phase, as a solid solution of inhibitor homogeneously distributed throughout the polymer or any combination of these states or those states that lie intermediate between them. Preferably, the dispersion is in the form of a “solid solution,” meaning that amorphous inhibitor is homogeneously distributed throughout the concentration-enhancing polymer, and that the compound present in relatively pure amorphous domains within the solid amorphous dispersion is relatively small, on the order of less than 20 weight percent, and preferably less than 10 wt percent of the total amount of inhibitor. Such solid solutions may also be termed substantially homogeneous. Solid solutions of amorphous inhibitor and polymer generally are more physically stable and have improved concentration-enhancing properties and, in turn improved bioavailability, relative to dispersions that are not solid solutions.

While the dispersion may have some inhibitor-rich domains, it is preferred that the dispersion itself have a single glass transition temperature (T_{g}) which demonstrates that the dispersion is substantially homogeneous. This contrasts with a simple physical mixture of pure amorphous 1-[5-(4-amino-7-isopropyl-7H-pyrrolo[2,3-d]pyrimidine-5-carbonyl)-2-methoxy-phenyl]-3-(2,4-dichloro-phenyl)-urea particles and pure amorphous polymer particles which generally display two distinct T_{g}s, one that of the inhibitor and one that of the polymer. T_{g} as used herein is the characteristic temperature where a glassy material, upon gradual heating, undergoes a relatively rapid (e.g., 10 to 100 seconds) physical change from a glass state to a rubber state. The T_{g} of an amorphous material such as a polymer, drug or dispersion can be measured by several techniques, including by a dynamic mechanical analyzer (DMA), a dilatometer, dielectric analyzer, and by a differential scanning calorimeter (DSC). The exact values measured by each technique can vary somewhat but usually fall within 10° to 30° C. of each other. Regardless of the technique used, when an amorphous dispersion exhibits a single T_{g}, this indicates that the dispersion is substantially homogenous. Dispersions of the present invention that are substantially homogeneous generally are more physically stable and have improved concentration-enhancing properties and, in turn improved bioavailability, relative to nonhomogeneous dispersions.

The compositions comprising the active agent and concentration-enhancing polymer provide enhanced concentration of the dissolved inhibitor in in vitro dissolution tests. It has been determined that enhanced drug concentration in in vitro dissolution tests in Model Fasted Duodenal (MFD) solution or Phosphate Buffered Saline (PBS) is a good indicator of in vivo performance and bioavailability. An appropriate PBS solution is an aqueous solution comprising 20 mM sodium phosphate (Na₂HPO₄), 47 mM potassium phosphate (KH₂PO₄), 87 mM sodium chloride (NaCl), and 0.2 mM potassium chloride (KCl), adjusted to pH 6.5 with sodium hydroxide (NaOH). An appropriate MFD solution is the same PBS solution wherein additionally is present 7.3 mM sodium taurocholic acid and 1.4 mM of 1-palmitoyl-2-oleyl-sn-glycero-3-phosphocholine. In particular, a composition of the present invention can be dissolution-tested by adding it to MFD or PBS solution and agitating to promote dissolution. Generally, the amount of composition added to the solution in such a test is an amount that, if all the drug in the composition dissolved, would produce an inhibitor concentration that is at least about 5 fold and preferably between 20 to 50 fold the equilibrium solubility of the active agent alone in the test solution. To demonstrate even higher levels of dissolved inhibitor concentration, addition of even larger amounts of the composition is desirable.

In one aspect, the compositions of the present invention provide a Maximum Drug Concentration (MDC) that is at least about 20-fold the equilibrium concentration of a control composition comprising an equivalent quantity of inhibitor but free from the polymer. In other words, if the equilibrium concentration provided by the control composition is 1 μg/mL, then a composition of the present invention provides an MDC of at least about 20 μg/mL. The control composition is conventionally the undispersed inhibitor alone (e.g., typically, the crystalline inhibitor alone in its most thermodynamically stable crystalline form) or the inhibitor plus a weight of inert diluent equivalent to the weight of polymer in the test composition. It is to be understood that the control composition is free from solubilizers or other components which would materially affect the solubility of the inhibitor, and that the inhibitor is in solid form in the control composition. Preferably, the MDC of inhibitor achieved with the compositions of the present invention is at least about 25 fold, more preferably at least about 20 fold, the equilibrium concentration of the control composition. Surprisingly, the present invention may achieve extremely large enhancements in aqueous concentration.

Alternatively, the compositions of the present invention provide in an aqueous use environment a concentration versus time Area Under The Curve (AUC), for any period of at least 90 minutes between the time of introduction into the use environment and about 270 minutes following introduction to the use environment, that is at least 10 fold that of a control composition comprising an equivalent quantity of undispersed inhibitor. Preferably, the compositions of the present invention provide in an aqueous use environment a concentration versus time AUC, for any period of at least 90 minutes between the time of introduction into the use environment and about 270 minutes following introduction to the use environment, that is at least about 20 fold, more preferably at least about 50 fold and even more preferably at least about 60 fold that of a control composition as described above. Such large enhancements in aqueous concentration versus time AUC values are surprising given the extremely low aqueous solubility and hydrophobicity of free base crystalline 1-[5-(4-amino-7-isopropyl-7H-pyrrolo[2,3-d]pyrimidine-5-carbonyl)-2-methoxy-phenyl]-3-(2,4-dichloro-phenyl)-urea.

As described above, the term “use environment” can be either the in vivo environment of the GI tract of a mammal, particularly a human, or the in vitro environment of a test solution, such as phosphate buffered saline (PBS) or Model Fasted Duodenal (MFD) solution.

A typical in vitro test to evaluate enhanced drug concentration in aqueous solution can be conducted by (1) adding with agitation a sufficient quantity of control composition, typically the inhibitor alone, to the in vitro test medium, typically MFD or PBS solution, to achieve equilibrium concentration of the inhibitor; (2) adding with agitation a sufficient quantity of test composition (e.g., the inhibitor and polymer) in an equivalent test medium, such that if all the inhibitor dissolved, the theoretical concentration of inhibitor would exceed the equilibrium concentration of the inhibitor by a factor of at least 5, and preferably a factor of at least 20; and (3) comparing the measured MDC and/or aqueous concentration versus time AUC of the test composition in the test medium with the equilibrium concentration, and/or the aqueous concentration versus time AUC of the control composition. In conducting such a dissolution test, the amount of test composition or control composition used is an amount such that if all of the inhibitor dissolved the inhibitor concentration would be at least 10 fold and preferably at least 50 fold that of the equilibrium concentration.

The concentration of dissolved inhibitor is typically measured as a function of time by sampling the test medium and plotting inhibitor concentration in the test medium vs. time so that the MDC can be ascertained. The MDC is taken to be the maximum value of dissolved inhibitor measured over the duration of the test. The aqueous concentration of the inhibitor versus time AUC is calculated by integrating the concentration versus time curve over any 90-minute time period between the time of introduction of the composition into the aqueous use environment (time equals zero) and 270 minutes following introduction to the use environment (time equals 270 minutes). Typically, when the composition reaches its MDC rapidly, less than about 30 minutes, the time interval used to calculate AUC is from time equals zero to time equals 90 minutes. However, if the AUC over any 90-minute time period described above of a composition meets the criterion of this invention, then the composition is a part of this invention.

A 0.36 mg free base active agent and 1.44 mg active agent in spray dry dispersion in 1.8 mL of MFD assayed according to the conditions described above yielded a MDC of 3 μg/mL and an AUC of 100 μg×min/mL and 80 μg/mL and an AUC of 5,900 μg×min/mL, respectively.

To avoid large inhibitor particulates which would give an erroneous determination, the test solution is either filtered or centrifuged. “Dissolved inhibitor” is typically taken as that material that either passes a 0.45 μm syringe filter or, alternatively, the material that remains in the supernatant following centrifugation. Filtration can be conducted using a 13 mm, 0.45 μm polyvinylidine difluoride syringe filter sold by Scientific Resources under the trademark TITAN®. Centrifugation is typically carried out in a polypropylene microcentrifuge tube by centrifuging at 13,000 G for 60 seconds. Other similar filtration or centrifugation methods can be employed and useful results obtained. For example, using other types of microfilters may yield values somewhat higher or lower (±10-40 percent) than that obtained with the filter specified above but will still allow identification of preferred dispersions. It is recognized that this definition of “dissolved inhibitor” encompasses not only monomeric solvated inhibitor molecules but also a wide range of species such as polymer/inhibitor assemblies that have submicron dimensions such as inhibitor aggregates, aggregates of mixtures of polymer and inhibitor, micelles, polymeric micelles, colloidal particles or nanocrystals, polymer/inhibitor complexes, and other such inhibitor containing species that are present in the filtrate or supernatant in the specified dissolution test.

Alternatively, the compositions of the present invention, when dosed orally to a human or other mammal, provide an AUC inhibitor concentration in the blood (herein referred to as a plasma AUC) that is at least about 4 fold that observed when a control composition comprising all equivalent quantity of undispersed drug is dosed. It is noted that such compositions can also be said to have a relative bioavailability of about 4. Preferably, the compositions of the present invention, when dosed orally to a human or other mammal, provide an AUC inhibitor concentration in the blood that is at least about 6 fold, more preferably at least about 10 fold, and even more preferably at least about 20 fold that observed when a control composition comprising an equivalent quantity of undispersed drug is dosed. It is to be understood that when dosed in vivo, the dosing vehicle does not contain any solubilizer or other components which would materially affect the solubility of the inhibitor, and that the inhibitor is in solid form in the control composition. An exemplary dosing vehicle would be a suspension solution of water containing 0.5 wt percent hydroxypropyl cellulose (such as METHOCEL) and 0.16 wt percent of the surfactant polyoxyethylene 20 sorbitan monooleate (such as TWEEN 80). Thus, the compositions of the present invention can be evaluated in either in vitro or in vivo tests, or both.

A spray dry dispersion prepared according to the methods of the invention has the plasma pharmacokinenetic properties described in Table 1 below.

TABLE 1

| | | Dose (mg | | | |
|---|---|---|---|---|---|
| | | Active | | | AUC₀₋₂₄ |
| Formulation | Species | Agent/kg) | T_{max.} (hr) | Cₘₐₓ (ng/mL) | (ng * h/mL) |
| Crystalline Freebase | Rat (n = 4) | 100 | 4.5 ± 1.0 | 191 ± 153 | 1300 ± 940 |
| 25 percent | Rat (n = 4) | 100 | 2.0 ± 0 | 9260 ± 2190 | 88900 ± 2880 |
| HPMCAS-HG SDD | | | | | |
| Crystalline Freebase | Dog (n = 3) | 30 | 4 ± 2 | 347 ± 556 | 2814 ± 4552 |
| 25 | Dog (n = 3) | 30 | 2.7 ± 1.2 | 2064 ± 969 | 13243 ± 5344 |
| percentHPMCAS-HG | | | | | |
| SDD | | | | | |

Relative bioavailability of inhibitor in the dispersions of the present invention can be tested in vivo in animals or humans using conventional methods for making such a determination. An in vivo test, such as a crossover study, may be used to determine whether a composition of inhibitor and concentration-enhancing polymer provides an enhanced relative bioavailability compared with a control composition comprised of inhibitor but no polymer as described above. In an in vivo crossover study a “test composition” of inhibitor and polymer is dosed to half a group of test subjects and, after an appropriate washout period (e.g., one week) the same subjects are dosed with a “control composition” that comprises an equivalent quantity of inhibitor as the “test composition” (but with no polymer present). The other half of the group is dosed with the control composition first, followed by the test composition. The relative bioavailability is measured as the concentration in the blood (serum or plasma) versus time area under the curve (AUC) determined for the test group divided by the AUC in the blood provided by the control composition. Preferably, this test/control ratio is determined for each subject, and then the ratios are averaged over all subjects in the study. In vivo determinations of AUC can be made by plotting the serum or plasma concentration of drug along the ordinate (y axis) against time along the abscissa (x-axis). It is to be understood by those skilled in the art that such in vivo tests are conventionally carried out under fasted conditions.

Thus, as noted above, one embodiment of the present invention is one in which the relative bioavailability of the test composition is at least about 4 relative to a control composition comprised of inhibitor but with no polymer as described above. (That is, the in vivo AUC provided by the test composition is at least about 4 fold the in vivo AUC provided by the control composition.) A preferred embodiment of the invention is one in which the relative bioavailability of the test composition is at least about 6, and even more preferably at least about 10 relative to a control composition composed of the inhibitor but with no polymer present, as described above. The determination of AUCs is a well-known procedure and is described, for example, in Welling, “Pharmacokinetics Processes and Mathematics,” ACS Monograph 185 (1986).

### Concentration-Enhancing Polymers

Concentration-enhancing polymers suitable for use in the compositions of the present invention should be inert, in the sense that they do not chemically react with 1-[5-(4-amino-7-isopropyl-7H-pyrrolo[2,3-d]pyrimidine-5-carbonyl)-2-methoxy-phenyl]-3-(2,4-dichloro-phenyl)-urea in an adverse manner, are pharmaceutically acceptable, and have at least some solubility in aqueous solution at physiologically relevant pHs (e.g., 1-8). The polymer can be neutral or ionizable, and should have an aqueous-solubility of at least 0.1 mg/mL over at least a portion of the pH range of 1-8.

The polymer is a “concentration-enhancing polymer,” meaning that it meets at least one, and more preferably both, of the following conditions. The first condition is that the concentration-enhancing polymer increases the MDC of 1-[5-(4-amino-7-isopropyl-7H-pyrrolo[2,3-d]pyrimidine-5-carbonyl)-2-methoxy-phenyl]-3-(2,4-dichloro-phenyl)-urea in the environment of use relative to a control composition consisting of an equivalent amount of 1-[5-(4-amino-7-isopropyl-7H-pyrrolo[2,3-d]pyrimidine-5-carbonyl)-2-methoxy-phenyl]-3-(2,4-dichloro-phenyl)-urea but no polymer. That is, once the composition is introduced into an environment of use, the polymer increases the aqueous concentration of 1-[5-(4-amino-7-isopropyl-7H-pyrrolo[2,3-d]pyrimidine-5-carbonyl)-2-methoxy-phenyl]-3-(2,4-dichloro-phenyl)-urea relative to the control composition. Preferably, the polymer increases the MDC of 1-[5-(4-amino-7-isopropyl-7H-pyrrolo[2,3-d]pyrimidine-5-carbonyl)-2-methoxy-phenyl]-3-(2,4-dichloro-phenyl)-urea in aqueous solution by at least 5-fold relative to a control composition, preferably by at least 15-fold, and more preferably by at least 50-fold. Such large enhancements may be necessary in order for 1-[5-(4-amino-7-isopropyl-7H-pyrrolo[2,3-d]pyrimidine-5-carbonyl)-2-methoxy-phenyl]-3-(2,4-dichloro-phenyl)-urea to achieve effective blood levels through oral dosing. The second condition is that the concentration-enhancing polymer increases the AUC of 1-[5-(4-amino-7-isopropyl-7H-pyrrolo[2,3-d]pyrimidine-5-carbonyl)-2-methoxy-phenyl]-3-(2,4-dichloro-phenyl)-urea in the environment of use relative to a control composition consisting of the active compound but where no polymer is present as described above. That is, in the environment of use, the composition comprising 1-[5-(4-amino-7-isopropyl-7H-pyrrolo[2,3-d]pyrimidine-5-carbonyl)-2-methoxy-phenyl]-3-(2,4-dichloro-phenyl)-urea and the concentration-enhancing polymer provides an area under the concentration versus time curve (AUC) for any period of 90 minutes between the time of introduction into the use environment and about 270 minutes following introduction to the use environment that is at least 10-fold that of a control composition comprising an equivalent quantity of the active agent but without polymer. Preferably, the AUC provided by the composition is at least 50-fold that of the control composition.

Concentration-enhancing polymers suitable for use in the present invention may be cellulosic or non-cellulosic. The polymers may be neutral or ionizable in aqueous solution. Of these, ionizable and cellulosic polymers are preferred, with ionizable cellulosic polymers being more preferred.

A preferred class of polymers comprises polymers that are “amphiphilic” in nature, meaning that the polymer has hydrophobic and hydrophilic portions. The hydrophobic portion may comprise groups such as aliphatic or aromatic hydrocarbon groups. The hydrophilic portion may comprise either ionizable or non-ionizable groups that are capable of hydrogen bonding such as hydroxyls, carboxylic acids, esters, amines or amides.

Amphiphilic and/or ionizable polymers are preferred because such polymers tend to have relatively strong interactions with 1-[5-(4-amino-7-isopropyl-7H-pyrrolo[2,3-d]pyrimidine-5-carbonyl)-2-methoxy-phenyl]-3-(2,4-dichloro-phenyl)-urea and may promote the formation of the various types of polymer/drug assemblies in the use environment as described previously. In addition, the repulsion of the like charges of the ionized groups of such polymers may serve to limit the size of the polymer/drug assemblies to the nanometer or submicron scale. For example, while not wishing to be bound by a particular theory, such polymer/drug assemblies may comprise hydrophobic clusters of the active agent surrounded by the polymer with the polymer's hydrophobic regions turned inward towards the active agent and the hydrophilic regions of the polymer turned outward toward the aqueous environment. Alternatively, the ionized functional groups of the polymer may associate, for example, via ion pairing or hydrogen bonds, with ionic or polar groups of the active agents. In the case of ionizable polymers, the hydrophilic regions of the polymer would include the ionized functional groups. Such polymer/drug assemblies in solution may well resemble charged polymeric micellar-like structures. In any case, regardless of the mechanism of action, the inventors have observed that such amphiphilic polymers, particularly ionizable cellulosic polymers, have been shown to improve the MDC and/or AUC of 1-[5-(4-amino-7-isopropyl-7H-pyrrolo[2,3-d]pyrimidine-5-carbonyl)-2-methoxy-phenyl]-3-(2,4-dichloro-phenyl)-urea in aqueous solution relative to control compositions free from such polymers.

Surprisingly, such amphiphilic polymers can greatly enhance the maximum concentration of 1-[5-(4-amino-7-isopropyl-7H-pyrrolo[2,3-d]pyrimidine-5-carbonyl)-2-methoxy-phenyl]-3-(2,4-dichloro-phenyl)-urea obtained when it is dosed to a use environment. In addition, such amphiphilic polymers interact with 1-[5-(4-amino-7-isopropyl-7H-pyrrolo[2,3-d]pyrimidine-5-carbonyl)-2-methoxy-phenyl]-3-(2,4-dichloro-phenyl)-urea to prevent the precipitation or crystallization of the active compound from solution despite its concentration being substantially above its equilibrium concentration. In particular, when the preferred compositions are solid amorphous dispersions of the 1-[5-(4-amino-7-isopropyl-7H-pyrrolo[2,3-d]pyrimidine-5-carbonyl)-2-methoxy-phenyl]-3-(2,4-dichloro-phenyl)-urea and the concentration-enhancing polymer, the compositions provide a greatly enhanced drug concentration, particularly when the dispersions are substantially homogeneous. The maximum drug concentration may be 5-fold and often more than 50-fold the equilibrium concentration of the crystalline 1-[5-(4-amino-7-isopropyl-7H-pyrrolo[2,3-d]pyrimidine-5-carbonyl)-2-methoxy-phenyl]-3-(2,4-dichloro-phenyl)-urea. Such enhanced concentrations of 1-[5-(4-amino-7-isopropyl-7H-pyrrolo[2,3-d]pyrimidine-5-carbonyl)-2-methoxy-phenyl]-3-(2,4-dichloro-phenyl)-urea in turn lead to substantially enhanced relative bioavailability for the active compound.

One class of polymers suitable for use with the present invention comprises neutral non-cellulosic polymers. Exemplary polymers include: vinyl polymers and copolymers having substituents of hydroxyl, alkylacyloxy, or cyclicamido; polyvinyl alcohols that have at least a portion of their repeat units in the unhydrolyzed (vinyl acetate) form; polyvinyl alcohol polyvinyl acetate copolymers; polyvinyl pyrrolidone; polyoxyethylene-polyoxypropylene copolymers, also known as poloxamers; and polyethylene polyvinyl alcohol copolymers.

Another class of polymers suitable for use with the present invention comprises ionizable non-cellulosic polymers. Exemplary polymers include: carboxylic acid-functionalized vinyl polymers, such as the carboxylic acid functionalized polymethacrylates and carboxylic acid functionalized polyacrylate such as the EUDRAGITS manufactured by Rohm Tech Inc., of Maiden, Mass.; amine-functionalized polyacrylates and polymethacrylates; proteins; and carboxylic acid functionalized starches such as starch glycolate.

Non-cellulosic polymers that are amphiphilic are copolymers of a relatively hydrophilic and a relatively hydrophobic monomer. Examples include acrylate and methacrylate copolymers, and polyoxyethylene-polyoxypropylene copolymers. Exemplary commercial grades of such copolymers include the EUDRAGITS, which are copolymers of methacrylates and acrylates, and the PLURONICS supplied by BASE, which are polyoxyethylene-polyoxypropylene copolymers.

A preferred class of polymers comprises ionizable and neutral cellulosic polymers with at least one ester- and/or ether-linked substituent in which the polymer has a degree of substitution of at least 0.1 for each substituent.

It should he noted that in the polymer nomenclature used herein, ether-linked substituents are recited prior to “cellulose” as the moiety attached to the ether group; for example, “ethylbenzoic acid cellulose” has ethoxybenzoic acid substituents. Analogously, ester-linked substituents are recited after “cellulose” as the carboxylate; for example, “cellulose phthalate” has one carboxylic acid of each phthalate moiety ester-linked to the polymer and the other carboxylic acid unreacted.

It should also be noted that a polymer name such as “cellulose acetate phthalate” (CAP) refers to any of the family of cellulosic polymers that have acetate and phthalate groups attached via ester linkages to a significant fraction of the cellulosic polymers hydroxyl groups. Generally, the degree of substitution of each substituent group can range from 0.1 to 2.9 as long as the other criteria of the polymer are met. “Degree of substitution” refers to the average number of the three hydroxyls per saccharide repeat unit on the cellulose chain that have been substituted. For example, if all of the hydroxyls on the cellulose chain have been phthalate substituted, the phthalate degree of substitution is 3. Also included within each polymer family type are cellulosic polymers that have additional substituents added in relatively small amounts that do not substantially alter the performance of the polymer.

Amphiphilic cellulosics comprise polymers in which the parent cellulosic polymer has been substituted at any or all of the 3 hydroxyl groups present on each saccharide repeat unit with at least one relatively hydrophobic substituent.

Hydrophobic substituents may be essentially any substituent that, if substituted to a high enough level or degree of substitution, can render the cellulosic polymer essentially aqueous insoluble. Examples of hydrophobic substituents include ether-linked alkyl groups such as methyl, ethyl, propyl, butyl, etc.; or ester-linked alkyl groups such as acetate, propionate, butyrate/-etc.; and ether- and/or ester-linked aryl groups such as phenyl, benzoate, or phenylate. Hydrophilic regions of the polymer can be either those portions that are relatively unsubstituted, since the unsubstituted hydroxyls are themselves relatively hydrophilic, or those regions that are substituted with hydrophilic substituents. Hydrophilic substituents include ether- or ester-linked nonionizable groups such as the hydroxy alkyl substituents hydroxyethyl, hydroxypropyl, and the alkyl ether groups such as ethoxyethoxy or methoxyethoxy. Particularly preferred hydrophilic substituents are those that are ether or ester-linked ionizable groups such as carboxylic acids, thiocarboxylic acids, substituted phenoxy groups, amines, phosphates or sulfonates.

One class of cellulosic polymers comprises neutral polymers, meaning that the polymers are substantially non ionizable in aqueous solution. Such polymers contain non ionizable substituents, which may be either ether-linked or ester-linked. Exemplary ether-linked non-ionizable substituents include: alkyl groups, such as methyl, ethyl, propyl, butyl, etc.; hydroxy alkyl groups such as hydroxymethyl, hydroxyethyl, hydroxypropyl, etc.; and aryl groups such as phenyl. Exemplary ester-linked non-ionizable substituents include: alkyl groups, such as acetate, propionate, butyrate, etc.; and aryl groups such as phenylate. However, when aryl groups are included, the polymer may need to include a sufficient amount of a hydrophilic substituent so that the polymer has at least some water solubility at any physiologically relevant pH of from 1 to 8.

Exemplary non-ionizable polymers that may be used as the polymer include: hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

A preferred set of neutral cellulosic polymers are those that are amphiphilic. Exemplary polymers include hydroxypropyl methyl cellulose and hydroxypropyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer.

A preferred class of cellulosic polymers comprises polymers that are at least partially ionizable at physiologically relevant pH and include at least one ionizable substituent, which may be either ether-linked or ester-linked.

Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid such as ethoxynicotinic acid, and the various isomers of picolinic acid such as ethoxypicolinic acid, etc.; thiocarboxylic acids, such as thioacetic acid; substituted phenoxy groups, such as hydroxyphenoxy, etc.; amines, such as aminoethoxy, diethylaminoethoxy, trimethylaminoethoxy, etc.; phosphates, such as phosphate ethoxy; and sulfonates, such as sulphonate ethoxy. Exemplary ester linked ionizable substituents include: carboxylic acids, such as succinate, citrate, phthalate, terephthalate, isophthalate, trimellitate, and the various isomers of pyridinedicarboxylic acid, etc.; thiocarboxylic acids, such as thiosuccinate; substituted phenoxy groups, such as amino salicylic acid; amines, such as natural or synthetic amino acids, such as alanine or phenylalanine; phosphates, such as acetyl phosphate; and sulfonates, such as acetyl sulfonate. For aromatic-substituted polymers to also have the requisite aqueous solubility, it is also desirable that sufficient hydrophilic groups such as hydroxypropyl or carboxylic acid functional groups be attached to the polymer to render the polymer aqueous soluble at least at pH values where any ionizable groups are ionized. In some cases, the aromatic group may itself be ionizable, such as phthalate or trimellitate substituents. Exemplary cellulosic polymers that are at least partially ionized at physiologically relevant pHs include: hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, carboxymethylethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate and ethyl picolinic acid cellulose acetate.

Exemplary ionizable cellulosic polymers that meet the definition of amphiphilic, having hydrophilic and hydrophobic regions, include polymers such as cellulose acetate phthalate and cellulose acetate trimellitate where the cellulosic repeat units that have one or more acetate substituents are hydrophobic relative to those that have no acetate substituents or have one or more ionized phthalate or trimellitate substituents.

A particularly desirable subset of cellulosic ionizable polymers are those that possess both a carboxylic acid functional aromatic substituent and an alkylate substituent and thus are amphiphilic. Exemplary polymers include cellulose acetate phthalate, methyl cellulose acetate phthalate, ethyl cellulose acetate phthalate, hydroxypropyl cellulose acetate phthalate, hydroxylpropyl methyl cellulose phthalate, hydroxypropyl methyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Another particularly desirable subset of cellulosic ionizable polymers are those that are amphiphilic and possess a non-aromatic carboxylate substituent. Exemplary polymers include hydroxypropyl methyl cellulose acetate succinate, hydroxypropyl methyl cellulose succinate, hydroxypropyl cellulose acetate succinate, hydroxyethyl methyl cellulose lo acetate succinate, hydroxyethyl methyl cellulose succinate, hydroxyethyl cellulose acetate succinate, and carboxymethyl ethyl cellulose.

While, as listed above, a wide range of polymers may be used to form dispersions of 1-[5-(4-amino-7-isopropyl-7H-pyrrolo[2,3-d]pyrimidine-5-carbonyl)-2-methoxy-phenyl]-3-(2,4-dichloro-phenyl)-urea, the inventors have found that relatively hydrophobic polymers have shown the best performance as demonstrated by high MDC and AUC values.

In particular, cellulosic polymers that are aqueous insoluble in their nonionized state but are at least sparingly aqueous soluble in their ionized state perform particularly well. A particular subclass of such polymers are the so-called enteric” polymers which include, for example, certain grades of hydroxypropyl methyl cellulose phthalate and cellulose acetate trimellitate. Dispersions formed from such polymers generally show very large enhancements, on the order of 5-fold to over 500-fold, in the maximum drug concentration achieved in dissolution tests relative to that for a crystalline drug control. In addition, non-enteric grades of such polymers as well as closely related cellulosic polymers are expected to perform well due to the similarities in physical properties with 1-[5-(4-amino-7-isopropyl-7H-pyrrolo[2,3-d]pyrimidine-5-carbonyl)-2-methoxy-phenyl]-3-(2,4-dichloro-phenyl)-urea.

Thus, especially preferred polymers are hydroxypropyl methyl cellulose acetate succinate (HPMCAS), hydroxypropyl methyl cellulose phthalate (HPMCP), cellulose acetate phthalate (CAP), cellulose acetate trimellitate (CAT), methyl cellulose acetate phthalate, hydroxypropyl cellulose acetate phthalate, cellulose acetate terephthalate cellulose acetate isophthalate, and carboxymethyl ethyl cellulose. The most preferred ionizable cellulosic polymers are hydroxypropyl methyl cellulose acetate succinate, hydroxypropyl methyl cellulose phthalate, cellulose acetate phthalate, cellulose acetate trimellitate, and carboxymethyl ethyl cellulose.

Hydroxypropyl methyl cellulose acetate succinate (HPMCAS) is available in several analytical grades and substituent profiles. Common commercial profiles for HPMCAS are described in the following Table. In addition to L, M and H grades, this cellulosic also is provided as a “G” grade which refers to a granule or pellet formulation or an “F” grade which refers to fine or powder formulation.

| | Composition of Shin Etsu's AQOAT | | |
|---|---|---|---|
| | Polymers (wt percent) | | |
| Substituent | L Grades | M Grades | H Grades |
| Methoxyl Content | 20.0-24.0 | 21.0-25.0 | 22.0-26.0 |
| Hydroxypropoxyl Content | 5.0-9.0 | 5.0-9.0 | 6.0-10.0 |
| Acetyl Content | 5.0-9.0 | 7.0-11.0 | 10.0-14.0 |
| Succinoyl Content | 14.0-18.0 | 10.0-14.0 | 4.0-8.0 |

One particularly effective polymer for forming dispersions of the present invention is carboxymethyl ethyl cellulose (CMEC). Dispersions made from 1-[5-(4-Amino-7-isopropyl-7H-pyrrolo[2,3-d]pyrimidine-5-carbonyl)-2-methoxy-phenyl]-3-(2,4-dichloro-phenyl)-urea and CMEC typically have high glass-transition temperatures at high relative humidities, due to the high glass-transition temperature of CMEC. As discussed below, such high T_{g}s result in solid amorphous dispersions with excellent physical stability. In addition, because all of the substituents on CMEC are attached to the cellulose backbone through ether linkages, CMEC has excellent chemical stability. Additionally, commercial grades of CMEC, such as that provided by Freund Industrial Company, Limited (Tokyo, Japan), are amphiphilic, leading to high degrees of concentration enhancement. Finally, 1-[5-(4-Amino-7-isopropyl-7H-pyrrolo[2,3-d]pyrimidine-5-carbonyl)-2-methoxy-phenyl]-3-(2,4-dichloro-phenyl)-urea has a high solubility in CMEC allowing for formation of physically stable dispersions with high drug loadings.

Another preferred class of polymers consists of neutralized acidic polymers. By “neutralized acidic polymer” is meant any acidic polymer for which a significant fraction of the “acidic moieties” or “acidic substituents” have been “neutralized”; that is, exist in their deprotonated form. By “acidic polymer” is meant any polymer that possesses a significant number of acidic moieties. In general, a significant number of acidic moieties would be greater than or equal to about 0.1 milliequivalents of acidic moieties per 30 gram of polymer. “Acidic moieties” include any functional groups that are sufficiently acidic that, in contact with or dissolved in water, can at least partially donate a hydrogen cation to water and thus increase the hydrogen-ion concentration. This definition includes any functional group or “substituent,” as it is termed when the functional group is covalently attached to a polymer that has a pKₐ of less than about 10. Exemplary classes of functional groups that are included in the above description include carboxylic acids, thiocarboxylic acids, phosphates, phenolic groups, and sulfonates. Such functional groups may make up the primary structure of the polymer such as for polyacrylic acid, but more generally are covalently attached to the backbone of the parent polymer and thus are termed “substituents.”

The “degree of neutralization,” α, of a polymer substituted with monoprotic acids (such as carboxylic acids) is defined as the fraction of the acidic moieties on the polymer that have been neutralized; that is, deprotonated by a base. Typically, for an acidic polymer to be considered a “neutralized acidic polymer,” α must be at least about 0.001 (or 0.1 percent), preferably about 0.01 (1 percent) and more preferably at least about 0.1 (10 percent). Such small degrees of neutralization may be acceptable because often the effective pH of the polymer changes dramatically with small increases in the degree of neutralization. Nonetheless, even greater degrees of neutralization are even more preferred. Thus, α is preferably at least 0.5 (meaning that at least 50 percent of the acidic moieties have been neutralized) and is more preferably at least 0.9 (meaning that at least 90 percent of the acidic moieties have been neutralized).

Neutralized acidic polymers are described in more detail in commonly assigned pending U.S. patent application Ser. No. 10/175,566 entitled “Pharmaceutical Compositions of Drugs and Neutralized Acidic Polymers” filed Jun. 17, 2002 and published as US 2003-0054038, the relevant disclosure of which is incorporated by reference.

When the neutralized form of the acidic polymer comprises a multivalent cationic species such as Ca²⁺, Mg²⁺, A³⁺, Fe²⁺, Fe³⁺, or a diamine, such as ethylene diamine, the cationic species may interact with two or more neutralized acidic moieties on more than one polymer chain, resulting in an ionic crosslink between the polymer chains. An acidic polymer may be considered “tonically crosslinked” if the number of milliequivalents of multivalent cationic species per gram of polymer is at least 5 percent preferably at least 10 percent the number of milliequivalents of acidic moieties (of the polymer) per gram of polymer. Alternatively, an acidic polymer may be considered “tonically crosslinked” if sufficient multivalent cationic species are present such that the neutralized acidic polymer has a higher T_{g} than the same polymer containing essentially no multivalent cationic species. Drug mobility in dispersions formed from such tonically cross-linked polymers is particularly low relative to dispersions formed from the acidic form of the same polymers. Such tonically crosslinked polymers may be formed by neutralization of the acidic polymer using any base where the cationic counterion of the base is divalent. Thus, calcium hydroxide, magnesium acetate or ethylene diamine may be added to an acidic polymer such as cellulosic acetate phthalate or hydroxypropyl methyl cellulose acetate succinate to form a neutralized, tonically crosslinked, acidic cellulosic polymer. Low drug mobility in such polymers may be indicated by high T_{g} values or, more typically, a decrease in the magnitude of the heat capacity increase in the vicinity of the T_{g} or, in some cases, the absence of any apparent T_{g} when the dispersion is subjected to differential thermal analysis. Thus, when the polymer is essentially completely neutralized, no T_{g} is apparent when the neutralized polymer is subjected to differential thermal analysis. Such tonically cross-linked polymers may provide improved physical stability for the drug in the dispersion relative to non-tonically crosslinked neutralized acidic polymer. While specific polymers have been discussed as being suitable for use in the compositions of the present invention, blends of such polymers may also be suitable. Thus the term “polymer” is intended to include blends of polymers in addition to a single species of polymer.

To obtain the best performance, particularly upon storage for long times prior to use, it is preferred that the active agent remain, to the extent possible, in the amorphous state. The inventors have found that this may best be achieved by two distinct methods. In the first method, the glass-transition temperature, T_{g}, of the amorphous 1-[5-(4-amino-7-isopropyl-7H-pyrrolo[2,3-d]pyrimidine-5-carbonyl)-2-methoxy-phenyl]-3-(2,4-dichloro-phenyl)-urea material is substantially above the storage temperature of the composition. In particular, it is preferable that the T_{g} of the amorphous state of 1-[5-(4-amino-7-isopropyl-7H-pyrrolo[2,3-d]pyrimidine-5-carbonyl)-2-methoxy-phenyl]-3-(2,4-dichloro-phenyl)-urea be at least 40° C. and preferably at least 60° C. For those aspects of the invention in which the composition is a solid, substantially amorphous dispersion of 1-[5-(4-amino-7-isopropyl-7H-pyrrolo[2,3-d]pyrimidine-5-carbonyl)-2-methoxy-phenyl]-3-(2,4-dichloro-phenyl)-urea in the concentration-enhancing polymer and in which the compound itself has a relatively low T_{g} (about 70° C. or less), it is preferred that the concentration-enhancing polymer have a T_{g} of at least 40° C., preferably at least 70° C. and more preferably greater than 100° C. Exemplary high T_{g} polymers include HPMCAS, HPMCP, CAP, CAT, CMEC and other cellulosics that have alkylate or aromatic substituents or both alkylate and aromatic substituents.

In a second method, the concentration-enhancing polymer is chosen such that the amorphous 1-[5-(4-Amino-7-isopropyl-7H-pyrrolo[2,3-d]pyrimidine-5-carbonyl)-2-methoxy-phenyl]-3-(2,4-dichloro-phenyl)-urea is highly soluble in the concentration-enhancing polymer. In general, the concentration-enhancing polymer and 1-[5-(4-Amino-7-isopropyl-7H-pyrrolo[2,3-d]pyrimidine-5-carbonyl)-2-methoxy-phenyl]-3-(2,4-dichloro-phenyl)-urea concentration are chosen such that the solubility of 1-[5-(4-Amino-7-isopropyl-7H-pyrrolo[2,3-d]pyrimidine-5-carbonyl)-2-methoxy-phenyl]-3-(2,4-dichloro-phenyl)-urea is roughly equal to or greater than the concentration of the active compound in the concentration-enhancing polymer. It is often preferred that the 1-[5-(4-Amino-7-isopropyl-7H-pyrrolo[2,3-d]pyrimidine-5-carbonyl)-2-methoxy-phenyl]-3-(2,4-dichloro-phenyl)-urea composition be chosen such that both methods—high T_{g} and high solubility—are satisfied.

In addition, the preferred polymers listed above, that is amphiphilic cellulosic polymers, tend to have greater concentration-enhancing properties relative to the other polymers of the present invention. For 1-[5-(4-amino-7-isopropyl-7H-pyrrolo[2,3-d]pyrimidine-5-carbonyl)-2-methoxy-phenyl]-3-(2,4-dichloro-phenyl)-urea, the amphiphilic cellulosic with the best concentration-enhancing properties are generally those that have ionizable substituents as well as hydrophobic substituents such as methoxy, ethoxy and acetate. In vivo tests of compositions with such polymers tend to have higher MDC and AUC values than compositions with other polymers of the invention.

### Preparation of Compositions

Dispersions of the active agent and concentration-enhancing polymer may be made according to any known process which results in at least a major portion (at least 60 percent) of the inhibitor being in the amorphous state. Exemplary mechanical processes include milling and extension; melt processes include high temperature fusion, solvent modified fusion and melt-congeal processes; and solvent processes include non-solvent precipitation, spray coating and spray-drying. See, for example, U.S. Pat. No. 5,456,923, U.S. Pat. No. 5,939,099 and U.S. Pat. No. 4,801,460 which describe formation of dispersions via extrusion processes; U.S. Pat. No. 5,340,591 and U.S. Pat. No. 4,673,564 which describe forming dispersions by milling processes; and U.S. Pat. No. 5,684,040 U.S. Pat. No. 4,894,235 and U.S. Pat. No. 5,707,646 which describe the formation of dispersions via melt/congeal processes, the disclosures of which are incorporated by reference. Although the dispersions of the present invention may be made by any of these processes, the dispersions generally have their maximum bioavailability and stability when the inhibitor is dispersed in the polymer such that it is substantially amorphous and substantially homogeneously distributed throughout the polymer.

In general, as the degree of homogeneity of the dispersion increases, the enhancement in the aqueous concentration of the active agent and relative bioavailability increases as well. Given the extremely low aqueous solubility and bioavailability of the freebase of 1-[5-(4-amino-7-isopropyl-7H-pyrrolo[2,3-d]pyrimidine-5-carbonyl)-2-methoxy-phenyl]-3-(2,4-dichloro-phenyl)-urea, is preferred for the dispersions to be as homogeneous as possible to achieve therapeutically effective levels of the inhibitor. Thus, most preferred are dispersions having a single glass transition temperature, which indicates a high degree of homogeneity. Dispersions with more than one Tg, indicating at least partial amorphous phase separation, may also function well, particularly when neither amorphous phase is comprised only of amorphous drug, but rather also contains a significant amount of concentration-enhancing polymer.

In one embodiment, the solid amorphous dispersion of inhibitor and concentration-enhancing polymer may be formed via a melt-congeal or melt-extrusion process. In such processes, a molten mixture comprising the inhibitor and concentration-enhancing polymer is rapidly cooled such that the molten mixture solidifies to form a solid amorphous dispersion. By “molten mixture” is meant that the mixture comprising the inhibitor and concentration-enhancing polymer is heated sufficiently that it becomes sufficiently fluid that the drug substantially disperses in one or more of the concentration-enhancing polymer and other excipients. Generally, this requires that the mixture be heated to about 10° C. or more above the lower of the melting point of the lowest melting point component in the composition and the melting point of the drug. The inhibitor can exist in the molten mixture as a pure phase, as a solution of inhibitor homogeneously distributed throughout the molten mixture, or any combination of these states or those states that lie intermediate between them. The molten mixture is preferably substantially homogeneous so that the inhibitor is dispersed as homogeneously as possible throughout the molten mixture. When the temperature of the molten mixture is below the melting point of both the inhibitor and the concentration-enhancing polymer, the molten excipients, concentration-enhancing polymer, and inhibitor are preferably sufficiently soluble in each other that a substantial portion of the inhibitor disperses in the concentration-enhancing polymer or excipients. It is often preferred that the mixture be heated above the lower of the melting point of the concentration-enhancing polymer and the inhibitor.

Generally, the processing temperature may vary from 50° C. up to about 200° C. or higher, depending on the melting point of the polymer, which is a function of the polymer grade selected. However, the processing temperature should not be so high that an unacceptably high level of degradation of the drug or polymer occurs. In some cases, the molten mixture should be formed under an inert atmosphere to prevent degradation of the drug and/or polymer at the processing temperature. When relatively high temperatures are used, it is often preferable to minimize the time that the mixture is at the elevated temperature to minimize degradation.

The molten mixture may also comprise an excipient that will reduce the melting temperature of the composition (either the drug and/or the polymer), allowing processing at lower temperature. When such excipients have low volatility and substantially remain in the mixture upon solidification, they generally can comprise up to 30 wt percent of the molten mixture. For example, a plasticizer may be added to the composition to reduce the melting temperature of the polymer. Examples of plasticizers include water, triethylcitrate, triacetin, and dibutyl sebacate. Volatile agents that dissolve or swell the polymer, such as acetone, water, methanol, and ethyl acetate, may also be added in low quantities to reduce the melting point of the composition. When such volatile excipients are added, at least a portion, up to essentially all, of such excipients may evaporate in the process of or following conversion of the molten mixture to a solid mixture. In such cases, the processing may be considered to be a combination of solvent processing and melt-congealing or melt-extrusion. Removal of such volatile excipients from the molten mixture can be accomplished by breaking up or atomizing the molten mixture into small droplets and contacting the droplets with a fluid such that the droplets both cool and lose all or part of the volatile excipient. Examples of other excipients that can be added to the composition to reduce the processing temperature include low molecular weight polymers or oligomers, such as polyethylene glycol, polyvinylpyrrolidone, and poloxamers, fats and oils, including mono-, di- and triglycerides; natural and synthetic waxes, such as carnauba wax, beeswax, microcrystalline wax, castor wax, and paraffin wax; long-chain alcohols, such as cetyl alcohol and stearyl alcohol; and long-chain fatty acids, such as stearic acid. As mentioned above, when the excipient added is volatile, it may be removed from the mixture while still molten or following solidification to form the solid amorphous dispersion.

Virtually any process may be used to form the molten mixture. One method involves melting the concentration-enhancing polymer in a vessel and then adding the active agent to the molten polymer. Another method involves melting the inhibitor in a vessel and then adding the concentration-enhancing polymer. In yet another method, a solid blend of the inhibitor and concentration-enhancing polymer may be added to a vessel and the blend heated to form the molten mixture.

Once the molten mixture is formed, it may be mixed to ensure the inhibitor is homogeneously distributed throughout the molten mixture. Such mixing may be done using mechanical means, such as overhead mixers, magnetically driven mixers and stir bars, planetary mixers, and homogenizers. Optionally, when the molten mixture is formed in a vessel, the contents of the vessel can be pumped out of the vessel and through an in-line or static mixer and then returned to the vessel. The amount of shear used to mix the molten mixture should be sufficiently high to ensure uniform distribution of the drug in the molten mixture. The molten mixture can be mixed from a few minutes to several hours, the mixing time being dependent on the viscosity of the mixture and the solubility of the drug and any optional excipients in the concentration-enhancing polymer.

An alternative method of preparing the molten mixture is to use two vessels, melting the inhibitor in the first vessel and the concentration-enhancing polymer in a second vessel. The two melts are then pumped through an in-line static mixer or extruder to produce the molten mixture that is then rapidly solidified.

Alternatively, the molten mixture can be generated using an extruder, such as a single-screw or twin-screw extruder, both well known in the art. In such devices, a solid feed of the composition is fed to the extruder whereby the combination of heat and shear forces produce a uniformly mixed molten mixture which can then be rapidly solidified to form the solid amorphous dispersion. The solid feed can be prepared using methods well known in the art for obtaining solid mixtures with high content uniformity. Alternatively, the extruder may be equipped with two feeders, allowing the inhibitor to be fed to the extruder through one feeder and the polymer through the other. Other excipients to reduce the processing temperature as described above may be included in the solid feed, or in the case of liquid excipients, such as water, may be injected into the extruder using methods well-known in the art.

The extruder should be designed such that it produces a molten mixture with the drug uniformly distributed throughout the composition. The various zones in the extruder should be heated to appropriate temperatures to obtain the desired extrudate temperature as well as the desired degree of mixing or shear, using procedures well known in the art.

When the active agent has a high solubility in the concentration-enhancing polymer, a lower amount of mechanical energy will be required to form the dispersion. In such cases, when the melting point of the undispersed inhibitor is greater than the melting point of the undispersed concentration-enhancing polymer, the processing temperature may be below the melting temperature of the undispersed inhibitor but greater than the melting point of the polymer, since the inhibitor will dissolve into the molten polymer. When the melting point of the undispersed inhibitor is less than the melting point of the undispersed concentration-enhancing polymer, the processing temperature may be above the melting point of the undispersed inhibitor but below the melting point of the undispersed concentration-enhancing polymer since the molten inhibitor will dissolve in the polymer or be absorbed into the polymer.

When the inhibitor has a low solubility in the polymer, a higher amount of mechanical energy may be required to form the dispersion. Here, the processing temperature may need to be above the melting point of the inhibitor and the polymer. As mentioned above, alternatively, a liquid or low-melting point excipient may be added that promotes melting or the mutual solubility of the concentration-enhancing polymer and inhibitor. A high amount of mechanical energy may also be needed to mix the inhibitor and the polymer to form a dispersion. Typically, the lowest processing temperature and an extruder design that imparts the lowest amount of mechanical energy (e.g., shear) that produces a satisfactory dispersion (substantially amorphous and substantially homogeneous) is chosen in order to minimize the exposure of the inhibitor to harsh conditions.

Once the molten mixture of inhibitor and concentration-enhancing polymer is formed, the mixture should be rapidly solidified to form the solid amorphous dispersion. By “rapidly solidified” is meant that the molten mixture is solidified sufficiently fast such that substantial phase separation of the drug and polymer does not occur. Typically, this means that the mixture should be solidified in less than about 10 minutes, preferably less than about 5 minutes, more preferably less than about 1 minute. If the mixture is not rapidly solidified, phase separation can occur, resulting in the formation of inhibitor-rich phases and polymer-rich phases. Over time, the active agent in the inhibitor-rich phases can crystallize. Such compositions are therefore not substantially amorphous or substantially homogeneous and tend not to perform as well as those compositions that are rapidly solidified and are substantially amorphous and substantially homogeneous. Solidification often takes place primarily by cooling the molten mixture to at least about 10° C. and preferably at least about 30° C. below its melting point. As mentioned above, solidification can be additionally promoted by evaporation of all or part of one or more volatile excipients or solvents. To promote rapid cooling and evaporation of volatile excipients, the molten mixture is often formed into a high surface area shape such as a rod or fiber or droplets. For example, the molten mixture can be forced through one or more small holes to form long thin fibers or rods or may be fed to a device, such as an atomizer, such as a rotating disk, that breaks the molten mixture up into droplets from 1 μm to 1 cm in diameter. The droplets are then contacted with a relatively cool fluid such as air or nitrogen to promote cooling and evaporation.

A useful tool for evaluating and selecting conditions for forming substantially homogeneous, substantially amorphous dispersions via a melt-congeal or extrusion process is the differential scanning calorimeter (DSC). While the rate at which samples can be heated and cooled in a DSC is limited, it does allow for precise control of the thermal history of a sample. For example, the inhibitor and concentration-enhancing polymer may be dry blended and then placed into the DSC sample pan. The DSC can then be programmed to heat the sample at the desired rate, hold the sample at the desired temperature for a desired time, and then rapidly cool the sample to ambient or lower temperature. The sample can then be re-analyzed on the DSC to verify the sample was transformed into a substantially homogeneous, substantially amorphous dispersion (e.g., the sample has a single Tg). Using this procedure, the temperature and time required to achieve a substantially homogeneous, substantially amorphous dispersion for the active agent and a given concentration-enhancing polymer can be determined.

The preferred method for forming substantially amorphous and substantially homogeneous dispersions is by “solvent processing,” which consists of dissolution of the inhibitor and one or more polymers in a common solvent. “Common” here means that the solvent, which can be a mixture of compounds, will simultaneously dissolve the drug and the polymer(s). After both the inhibitor and the polymer have been dissolved, the solvent is rapidly removed by evaporation or by mixing with a non-solvent. Exemplary processes are spray-drying, spray-coating (pan-coating, fluidized bed coating, etc.), and precipitation by rapid mixing of the polymer and drug solution with CO₂, water, or some other non-solvent. Preferably, removal of the solvent results in a solid dispersion which is substantially homogeneous. As described previously, in such substantially homogeneous dispersions, the inhibitor is dispersed as homogeneously as possible throughout the polymer and can be thought of as a solid solution of inhibitor dispersed in the polymer(s). When the resulting dispersion constitutes a solid solution of inhibitor in polymer, the dispersion may be thermodynamically stable, meaning that the concentration of inhibitor in the polymer is at or below its equilibrium value, or it may be considered a supersaturated solid solution where the inhibitor concentration in the dispersion polymer(s) is above its equilibrium value.

The solvent may be removed through the process of spray-drying. The term spray-drying is used conventionally and broadly refers to processes involving breaking up liquid mixtures into small droplets (atomization) and rapidly removing solvent from the mixture in a container (spray-drying apparatus) where there is a strong driving force for evaporation of solvent from the droplets. The strong driving force for solvent evaporation is generally provided by maintaining the partial pressure of solvent in the spray-drying apparatus well below the vapor pressure of the solvent at the temperature of the drying droplets. This is accomplished by either (1) maintaining the pressure in the spray-drying apparatus at a partial vacuum (e.g., 0.01 to 0.50 atm); (2) mixing the liquid droplets with a warm drying gas; or (3) both. In addition, at least a portion of the heat required for evaporation of solvent may be provided by heating the spray solution.

Solvents suitable for spray-drying can be any organic compound in which the inhibitor and polymer are mutually soluble. Preferably, the solvent is also volatile with a boiling point of 150° C. or less. In addition, the solvent should have relatively low toxicity and be removed from the dispersion to a level that is acceptable according to The International Committee on Harmonization (ICH) guidelines. Removal of solvent to this level may require a processing step such as tray-drying subsequent to the spray-drying or spray-coating process. Preferred solvents include alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, and 1,1,1-trichloroethane. Lower volatility solvents such as dimethyl acetamide or dimethylsulfoxide can also be used. Mixtures of solvents such as 50 percent methanol and 50 percent acetone, can also be used, as can mixtures with water as long as the polymer and inhibitor are sufficiently soluble to make the spray-drying process practicable. Generally, due to the hydrophobic nature of the inhibitor, non-aqueous solvents are preferred meaning that the solvent comprises less than about 10 wt percent water, and preferably less than 1 wt percent water.

Generally, the temperature and flow rate of the drying gas is chosen so that the polymer/drug-solution droplets are dry enough by the time they reach the wall of the apparatus that they are essentially solid, and so that they form a fine powder and do not stick to the apparatus wall. The actual length of time to achieve this level of dryness depends on the size of the droplets. Droplet sizes generally range from 1 μm to 500 μm in diameter, with 5 μm to 100 μm being more typical. The large surface-to-volume ratio of the droplets and the large driving force for evaporation of solvent leads to actual drying times of a few seconds or less, and more typically less than 0.1 second. This rapid trying is often critical to the particles maintaining a uniform, homogeneous dispersion instead of separating into drug-rich and polymer-rich phases. As above, to get large enhancements in concentration and bioavailability it is often necessary to obtain as homogeneous of a dispersion as possible. Solidification times should be less than 100 seconds, preferably less than a few seconds, and more preferably less than 1 second. In general, to achieve this rapid solidification of the inhibitor/polymer solution, it is preferred that the size of droplets formed during the spray-drying process is less than about 100 μm in diameter. The resultant solid particles thus formed are generally less than about 100 μm in diameter.

Following solidification, the solid powder typically stays in the spray-drying chamber for about 5 to 60 seconds, further evaporating solvent from the solid powder. The final solvent content of the solid dispersion as it exits in the dryer should be low, since this reduces the mobility of inhibitor molecules in the dispersion, thereby improving its stability. Generally, the solvent content of the dispersion as it leaves the spray-drying chamber should be less than 10 wt percent and preferably less than 2 wt percent. In some cases, it may be preferable to spray a solvent or a solution of a polymer or other excipient into the spray-drying chamber to form granules, so long as the dispersion is not adversely affected.

Spray-drying processes and spray-drying equipment are described generally in Perry's Chemical Engineers' Handbook, Sixth Edition (R. H. Perry, D. W. Green, J. O. Maloney, eds.) McGraw Hill Book Co. 1984, pages 2054 to 2057. More details on spray-drying processes and equipment are reviewed by Marshall “Atomization and Spray-Drying,” 50 Chem. Eng. Prog. Monogr. Series 2 (1954).

The amount of concentration-enhancing polymer relative to the amount of inhibitor present in the dispersions of the present invention depends on the inhibitor and polymer and may vary widely from a inhibitor-to-polymer weight ratio of from 0.01 to about 4 (e.g., 1 wt percent inhibitor to 80 wt percent inhibitor). However, in most cases it is preferred that the inhibitor-to-polymer ratio is greater than about 0.05 (4.8 wt percent inhibitor) and less than about 2.5 (71 wt percent inhibitor). Often the enhancement in inhibitor concentration or relative bioavailability that is observed increases as the inhibitor-to-polymer ratio decreases from a value of about 1 (50 wt percent inhibitor to a value of about 0.11 (10 wt percent inhibitor). In some cases it has been found that the bioavailability of dispersions with a inhibitor-to-polymer ratio of about 0.33 (25 wt percent inhibitor) have higher bioavailability when dosed orally than dispersions with a inhibitor-to-polymer ratio of 0.11 (10 wt percent inhibitor).

In addition, the amount of concentration-enhancing polymer that can be used in a dosage form is often limited by the total mass requirements of the dosage form. For example, when oral dosing to a human is desired, at low inhibitor to-polymer ratios, the total mass of drug and polymer may be unacceptably large for delivery of the desired dose in a single tablet or capsule. Thus, it is often necessary to use inhibitor-to-polymer ratios that are less than optimum in specific dosage forms to provide a sufficient inhibitor dose in a dosage form that is small enough to be easily delivered to a use environment.

### Excipients and Dosage Forms

Although the key ingredients present in the compositions of the present invention are simply the inhibitor 1-[5-(4-amino-7-isopropyl-7H-pyrrolo[2,3-d]pyrimidine-5-carbonyl)-2-methoxy-phenyl]-3-(2,4-dichloro-phenyl)-urea and the concentration-enhancing polymer(s), the inclusion of other excipients in the composition may be useful. These excipients may be utilized with the inhibitor and polymer composition in order to formulate the composition into tablets, capsules, suspensions, powders for suspension, creams, transdermal patches, depots, and the like. The composition of inhibitor and polymer can be added to other dosage form ingredients in essentially any manner that does not substantially alter the inhibitor. The excipients may be either physically mixed with the dispersion and/or included within the dispersion.

One very useful class of excipients is surfactants. Suitable surfactants include fatty acid and alkyl sulfonates; commercial surfactants such as benzalkonium chloride (HYAMINE® 1622, available from Lonza, Inc., Fairlawn, N.J.); dioctyl sodium sulfosuccinate, DOCUSATE SDDIUM® (available from Mallinckrodt Spec. Chem., St. Louis, Mo.); polyoxyethylene sorbitan fatty acid esters (TWEEN®, available from ICI Americas Inc., Wilmington, Del.; LIPOSORB® P-20 available from Lipochem Inc., Patterson, N.J.; CAPMUL® POE-0 available from Abitec Corp., Janesville, Wis.), and natural surfactants such as sodium taurocholic acid, 1 palmitoyl-2-oleoyl-sn-glycero-3-phosphocholine, lecithin, and other phospholipids and mono- and diglycerides. Such materials can advantageously be employed to increase the rate of dissolution by facilitating wetting, thereby increasing the maximum dissolved concentration, and also to inhibit crystallization or precipitation of drug by interacting with the dissolved drug by mechanisms such as complexation, formation of inclusion complexes, formation of micelles or adsorbing to the surface of solid drug, crystalline or amorphous. These surfactants may comprise up to about 5 wt percent of the composition.

The addition of pH modifiers such as acids, bases, or buffers may also be beneficial, retarding the dissolution of the composition (e.g., acids such as citric acid or succinic acid when the concentration-enhancing polymer is anionic) or, alternatively, enhancing the rate of dissolution of the composition (e.g., bases such as sodium acetate or amines when the polymer is anionic).

Conventional matrix materials, complexing agents, solubilizers, fillers, disintegrating agents (disintegrants), or binders may also be added as part of the composition itself or added by granulation via wet or mechanical or other means. These materials may comprise up to 90 wt percent of the composition.

Examples of matrix materials, fillers, or diluents include lactose, mannitol, xylitol, microcrystalline cellulose, calcium diphosphate, and starch.

Examples of disintegrants include sodium starch glycolate, sodium alginate, carboxy methyl cellulose sodium, methyl cellulose, and croscarmellose sodium.

Examples of binders include methyl cellulose, microcrystalline cellulose, starch, and gums such as guar gum, and tragacanth.

Examples of lubricants include magnesium stearate and calcium stearate.

Other conventional excipients may be employed in the compositions of this invention, including those excipients well-known in the art. Generally, excipients such as pigments, lubricants, flavorants, and so forth may be used for customary purposes and in typical amounts without adversely affecting the properties of the compositions. These excipients may be utilized in order to formulate the composition into tablets, capsules, suspensions, powders for suspension, creams, transdermal patches, and the like.

The compositions of the present invention may be delivered by a wide variety of routes, including, but not limited to, oral, nasal, rectal, and pulmonary. Generally, the oral route is preferred.

Compositions of this invention may also be used in a wide variety of dosage forms for administration of anticancer compounds. Exemplary dosage forms are powders or granules that may be taken orally either dry or reconstituted by addition of water or other liquids to form a paste, slurry, suspension or solution; tablets; capsules; multiparticulates; and pills. Various additives may be mixed, ground, or granulated with the compositions of this invention to form a material suitable for the above dosage forms.

The compositions of the present invention may be formulated in various forms such that they are delivered as a suspension of particles in a liquid vehicle. Such suspensions may be formulated as a liquid or paste at the time of manufacture, or they may be formulated as a dry powder with a liquid, typically water, added at a later time but prior to oral administration. Such powders that are constituted into a suspension are often termed sachets or oral powder for constitution (OPC) formulations. Such dosage forms can be formulated and reconstituted via any known procedure. The simplest approach is to formulate the dosage form as a dry powder that is reconstituted by simply adding water and agitating. Alternatively, the dosage form may be formulated as a liquid and a dry powder that are combined and agitated to form the oral suspension. In yet another embodiment, the dosage form can be formulated as two powders which are reconstituted by first adding water to one powder to form a solution to which the second powder is combined with agitation to form the suspension.

Generally, it is preferred that the dispersion of the active agent be formulated for long-term storage in the dry state as this promotes the chemical and physical stability of the inhibitor. Various excipients and additives are combined with the compositions of the present invention to form the dosage form. For example, it may be desirable to add some or all of the following preservatives such as sulfites (an antioxidant), benzalkonium chloride, methylparaben, propylparaben, benzyl alcohol or sodium benzoate; suspending agents or thickeners such as xanthan gum, starch, guar gum, sodium alginate, carboxymethyl cellulose, sodium carboxymethyl cellulose, methyl cellulose, hydroxypropyl methyl cellulose, polyacrylic acid, silica gel, aluminum silicate, magnesium silicate, or titanium dioxide; anticaking agents or fillers such as silicon oxide, or lactose; flavorants such as natural or artificial flavors; sweeteners such as sugars such as sucrose, lactose, or sorbitol as well as artificial sweeteners such as aspartame or saccharin; wetting agents or surfactants such as various grades of polysorbate, docusate sodium, or sodium lauryl sulfate; solubilizers such as ethanol propylene glycol or polyethylene glycol; coloring agents such as FD and C Red No. 3 or FD and C Blue No. 1; and pH modifiers or buffers such as carboxylic acids (including citric acid, ascorbic acid, lactic acid, and succinic acid), various salts of carboxylic acids, amino acids such as glycine or alanine, various phosphate, sulfate and carbonate salts such as trisodium phosphate, sodium bicarbonate or potassium bisulfate and bases such as amino glucose or triethanol amine.

A preferred additive to such formulations is additional concentration-enhancing polymer which may act as a thickener or suspending agent as well as to enhance the concentration of the inhibitor in the environment of use and may also act to prevent or retard precipitation or crystallization of the inhibitor from solution. Such preferred additives are hydroxyethyl cellulose, hydroxypropyl cellulose, and hydroxypropyl methyl cellulose. In particular, the salts of carboxylic acid functional polymers such as cellulose acetate phthalate, hydroxypropyl methyl cellulose acetate succinate, and carboxymethyl cellulose are useful in this regard. Such polymers may be added in their salt forms or the salt form may be formed in situ during reconstitution by adding a base such as trisodium phosphate and the acid form of such polymers.

In some cases, the overall dosage form or particles, granules or beads that make up the dosage form may have superior performance if coated with an enteric polymer to prevent or retard dissolution until the dosage form leaves the stomach. Exemplary enteric coating materials include hydroxypropyl methyl cellulose acetate succinate, hydroxypropyl methyl cellulose phthalate, cellulose acetate phthalate, cellulose acetate trimellitate, carboxylic acid-functionalized polymethacrylates, and carboxylic acid-functionalized polyacrylate.

Compositions of this invention may be administered in a controlled release dosage form. In one such dosage form, the composition of the inhibitor and polymer is incorporated into an erodible polymeric matrix device. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible polymeric matrix imbibes water and forms an aqueous-swollen gel or “matrix” that entraps the dispersion of inhibitor and polymer. The aqueous-swollen matrix gradually erodes, swells, disintegrates or dissolves in the environment of use, thereby controlling the release of the dispersion to the environment of use. Examples of such dosage forms are disclosed more fully in commonly assigned pending U.S. patent application Ser. No. 09/495,059 filed Jan. 31, 2000 which claimed the benefit of priority of Provisional Patent Application Ser. No. 60/119,400, filed Feb. 10, 1999, the relevant disclosure of which is herein incorporated by reference.

Alternatively, the compositions of the present invention may be administered by or incorporated into a non-erodible matrix device.

Alternatively, the compositions of the invention may be delivered using a coated osmotic-controlled release dosage form. This dosage form has two components: (a) the core which contains an osmotic agent and the dispersion of the active agent and concentration-enhancing polymer; and (b) a non-dissolving and non-eroding coating surrounding the core, the coating controlling the influx of water to the core from an aqueous environment of use so as to cause drug release by extension of some or all of the core to the environment of use. The osmotic agent contained in the core of this device may be an aqueous-swellable hydrophilic polymer, osmogen, or osmagent. The coating is preferably polymeric, aqueous-permeable, and has at least one delivery port. Examples of such dosage forms are disclosed more fully in commonly assigned pending U.S. patent application Ser. No. 09/495,061 filed Jan. 31, 2000 which claimed the benefit of priority of Provisional Patent Application Ser. No. 60/119,406, filed Feb. 10, 1999, the relevant disclosure of which is herein incorporated by reference.

Alternatively, the compositions may be delivered via a coated hydrogel-controlled release form having at least two components: (a) a core comprising the dispersion of the present invention and a hydrogel, and (b) a coating through which the dispersion has passage when the dosage form is exposed to a use environment. Examples of such dosage forms are more fully disclosed in commonly assigned European Patent EP0378404, the relevant disclosure of which is herein incorporated by reference.

Alternatively, the drug mixture of the invention may be delivered via a coated hydrogel-controlled release dosage form having at least three components: (a) a composition containing the dispersion, (b) a water-swellable composition wherein the water-swellable composition is in a separate region within a core formed by the drug-containing composition and the water-swellable composition, and (c) a coating around the core that is water-permeable, water-insoluble, and has at least one delivery port therethrough. In use, the core imbibes water through the coating, swelling the water-swellable composition and increasing the pressure within the core, and fluidizing the dispersion containing composition. Because the coating remains intact, the dispersion-containing composition is extruded out of the delivery port into an environment of use. Examples of such dosage forms are more fully disclosed in commonly assigned pending U.S. patent application Ser. No. 09/745,095, filed Dec. 20, 2000, which claims priority to Provisional Application Ser. No. 60/171,968, filed Dec. 23, 1999, the relevant disclosure of which is herein incorporated by reference.

Alternatively, the compositions may be administered as multiparticulates. Multiparticulates generally refer to dosage forms that comprise a multiplicity of particles that may range in size from about 10 μm to about 2 mm, more typically about 100 μm to 1 mm in diameter. Such multiparticulates may be packaged, for example, in a capsule such as a gelatin capsule or a capsule formed from an aqueous-soluble polymer such as HPMCAS, HPMC or starch or they may be dosed as a suspension or slurry in a liquid.

Such multiparticulates may be made by any known process, such as wet- and dry-granulation processes, extrusion/spheronization, roller-compaction, or by spray-coating seed cores. For example, in wet- and dry-granulation processes, the composition of the inhibitor and concentration-enhancing polymer is prepared as described above. This composition is then granulated to form multiparticulates of the desired size. Other excipients, such as a binder (e.g., microcrystalline cellulose), may be blended with the composition to aid in processing and forming the multiparticulates. In the case of wet granulation, a binder such as microcrystalline cellulose may be included in the granulation fluid to aid in forming a suitable multiparticulate.

In any case, the resulting particles may themselves constitute the multiparticulate dosage form or they may be coated by various film-forming materials such as enteric polymers or water-swellable or water-soluble polymers, or they may be combined with other excipients or vehicles to aid in dosing to patients.

### Methods of Treatment and Testing

One aspect of this invention is directed to a method for treating hyperproliferative diseases, such as cancers so a mammal (including a human being) by administering to a mammal in need of such treatment an anti-hyperproliferative effective amount of a composition of the present invention.

The crystalline and non-crystalline forms and formulations of 1-[5-(4-Amino-7-isopropyl-7H-pyrrolo[2,3-d[pyrimidine-5-carbonyl)-2-methoxy-phenyl]-3-(2 4-dichloro-phenyl)-urea are selective inhibitors of the tyrosine kinases, Tie-2, TrkA and related family member TrkB. The potentcy of these forms and formulations of the present invention at the tyrosine kinases may be determined using the following assays. 1-[5-(4-amino-7-isopropyl-7H-pyrrolo[2,3-d]pyrimidine-5-carbonyl)-2-methoxy-phenyl]-3-(2,4-dichloro-phenyl)-urea has an IC₅₀ of 144 ng/mL utilizing a fully activated recombinant GST-TIE2 construct. Assessment of the inhibition of the inactive (unphosphorylated) TIE2 kinase by 1-[5-(4-amino-7-isopropyl-7H-pyrrolo[2,3-d]pyrimidine-5-carbonyl)-2-methoxy-phenyl]-3-(2,4-dichloro-phenyl)-urea resulted in a Ki value of 11.8 ng/mL. In a cell-based assay, 1-[5-(4-amino-7-isopropyl-7H-pyrrolo[2,3-d]pyrimidine-5-carbonyl)-2-methoxy-phenyl]-3-(2,4-dichloro-phenyl)-urea inhibits the TIE-2 kinase activity of a recombinant human erbB-TIE2 chimeric receptor (erbB extracellular domain with TIE2 intracellular domain) that was overexpressed in C6 rat glioma cells (IC₅₀ equals 2.4 ng/mL) see Schindler, et al., 2000; Noble, et al., 2004.

The in vitro activity of the active agent in inhibiting the Tie-2 receptor may be determined by the following procedure.

Inhibition of Tie-2 tyrosine kinase activity is measured in 96-well Maxisorp plates (Nunc) coated with poly-Glu-Tyr (PGT 4:1 Sigma) by the addition of 100 μL/well of a 25 μg/mL solution of PGT in PBS. Plates are incubated at 37° C. overnight, and transferred to 4° C. until use. Prior to compound testing, appropriate dilutions of compounds are made in 96-well polypropylene plates. The active agents could then be diluted to 60-fold the desired final concentrations in DMSO, and subsequently diluted to 4-fold the desired final concentrations in phosphorylation buffer-DTT (PB-DTT), a buffer composed of 50 mM HEPES, pH 7.4, 125 mM NaCl, 24 mM MgCl₂, and 2 mM of freshly added dithiothreitol (DTT; Sigma). The PGT-coated plates are removed from 4° C., and washed 5 times with TBST, a wash buffer composed of 1× Tris-buffered saline made from powder (Sigma) containing 0.1 percent polyoxyethylenesorbitan monolaurate (Tween-20, Sigma). Twenty-five μL of each form or formulation of the active agent dilution per well could then be added to the washed PGT-coated plate. Plates could then receive 50 μL/well of a solution of 200 mM ATP (Sigma), freshly diluted in PB-DTT from a frozen 50 mM stock solution. Control wells could receive 50 μL/well PB-DTT lacking ATP. Reactions would then be initiated by the addition of 25 μL of purified GST-Tie-2 fusion protein in PB-DTT. GST-Tie-2 would be previously isolated from insect cells infected with GST-Tie-2 baculoviruses, and used at concentrations determined to provide OD₄₅₀ signals of approximately 1.0 in the presence of ATP and the absence of chemical inhibitors. Reactions would then be allowed to proceed for 15 minutes at ambient temperatures with shaking, and terminated by washing 5 times with TBST. To detect phosphotyrosine, the wash buffer would be removed, and each well receives 75 μL of a horseradish peroxidase-conjugated monoclonal antibody to phosphotyrosine (HRP-PY20; Signal Transduction Labs), diluted 1:2000 in block buffer, a buffer composed of wash buffer and 5 percent bovine serum albumin (BSA: Sigma). Plates would be incubated for 30 minutes with shaking at ambient temperature, and washed 5 times with wash buffer. The bound HRP-PY20 antibody can be detected by the addition of 70 μL/well TMB microwell substrate (KPL), and color development terminated by the addition of an equal volume of 0.9 M H₂SO₄. The background signal from wells lacking ATP can be subtracted from all ATP-stimulated wells, and IC₅₀ values calculated.

The cell assay utilizes NIH/3T3 fibroblasts expressing a chimeric receptor composed of the extracellular domain of the human EGFR, and the intracellular domain of human Tie-2. To measure cellular activity, fifteen thousand cells are seeded into 96-well U-bottom plates (Falcon) in Dulbecco's Modified Essential Medium (DMEM) containing 2 mM L-glutamine, 0.1 U/mL penicillin, 0.1 μg/mL streptomycin and 10 percent fetal calf serum (FCS; all supplements from Gibco). Cells are allowed to attach for six hours at 37° C. 5 percent CO₂, at which time the medium is replaced with 190 μL/well starvation medium (fresh medium containing 0.1 percent FCS). The cell plates are returned to the incubator until the next day. Prior to compound testing, appropriate dilutions of compounds are made in 96-well polypropylene plates. The initial dilution series begins with the addition of 15 μL of a 4 mM compound stock solution in DMSO to 45 μL DMSO; the resulting concentration of 1 mM is diluted in a serial 1:4 fashion in DMSO to give concentrations of 1000, 250, 62.5, 15.63. 3.91, 0.98. 0.25 and 0 μM. In a separate 96-well plate, 20 μL of each compound dilution is then added to 80 μL of starvation medium to give compound concentrations of 200, 50, 12.5, 3.13, 0.78, 0.20, 0.049 and 0 μM. In a final DMSO concentration of 20 percent. To dose cells, 10 μL of the various compound dilutions are added to the plates containing cells, to give final compound concentrations of 10, 2.5, 0.63, 0.16, 0.039, 0.01, 0.002 and 0 μM in 1 percent DMSO. Cell plates are allowed to incubate with compounds for 60 minutes at 37° C., 5 percent CO₂. To activate the chimeric receptors, recombinant EGF (Sigma) is added to a final concentration of 200 ng/mL, and plates are incubated for an additional 10 minutes at 37° C., 5 percent CO₂. Medium is then removed, and the cells are fixed for 5 minutes on ice with 100 μL/well cold methanol containing 200 μM NaVO₄. The fixative is removed and plates are allowed to dry at ambient temperature. Phosphotyrosine levels are measured in a time-resolved immunoassay with DELFIA Eu-N¹-labeled Anti-Phosphotyrosine Antibody (PT66) from Perkin Elmer™. The antibody is diluted to a final concentration of 0.5 μg/mL in DELFIA Assay Buffer (Perkin Elmer™), and 100 μL/well is added for 60 minutes at ambient temperature with shaking. The antibody solution is removed, and plates are washed six times using 300 μL/well DELFIA Wash Buffer (Perkin Elmer™). After the final wash, 100 μL/well of DELFIA Enhancement Solutiton (Perkin Elmer™) is added to each well. The DELFIA Enhancement Solution (Perkin Elmer™) acts to dissociate the Europium ions, which form highly fluorescent chelates. After incubation at ambient temperatures for 5 minutes with shaking, the plates are read on a Victor 2 Multilabel HTS Counter (Perkin Elmer™). The background signal from mock-stimulated wells is subtracted from the EGF-stimulated wells, and IC₅₀ values are calculated.

The in vitro activity of the crystalline and non-crystalline forms and formulations of 1-[5-(4-Amino-7-isopropyl-7H-pyrrolo[2,3-d]pyrimidine-5-carbonyl)-2-methoxy-phenyl]-3-(2,4-dichloro-phenyl)-urea in inhibiting the TrkA receptor may be determined by the following procedure.

The ability of the forms and formulations of the active agent of the present invention to inhibit tyrosine kinase activity of TrkA may be measured using a recombinant enzyme in an assay that measures the ability of compounds to inhibit the phosphorylation of the exogenous substrate, polyGluTyr (PGT, Sigma™, 4:1). The kinase domain of the human NGF/TrkA receptor is expressed in Sf9 insect cells as a glutathione S-transferase (GST)-fusion protein using the baculovirus expression system. The protein is purified from the lysates of these cells using glutathione agarose affinity columns. The enzyme assay is performed in 96-well plates that are coated with the PGT substrate (1.0 ug PGT per well). The final concentration of ATP in the plates is 40 uM. Test forms and formulations of the active agent are first diluted in dimethylsulfoxide (DMSO) and then serial-diluted in a 96-well plate. When added to the PGT plates, the final concentration of DMSO in the assay is 0.06 percent. The recombinant enzyme is diluted in phosphorylation buffer (50 mM HEPES pH 7.4. 0.14M NaCl, 2.2 mM MgCl₂, 2.5 mM MnCl₂, 0.1 mM DTT, 0.2 mM Na₃VO₄). The reaction is initiated by the addition of the recombinant enzyme to the ATP and to the test compounds. After a 30-minute incubation at room temperature with shaking, the reaction is stopped with 0.5M EDTA, pH 8.0, and then aspirated. The plates are washed with wash buffer (1× imidazole wash buffer). The amount of phosphorylated PGT is quantitated by incubation with a HRP-conjugated (HRP is horseradish peroxidase) PY-54 antibody (Transduction Labs), developed with ABTS substrate, and the reaction is quantitated on a Wallac Vistor² plate reader at 405 nm. Inhibition of the kinase enzymatic activity by the test compound is detected as a reduced absorbance, and the concentration of the compound that is required to inhibit the signal by 50 percent is reported as the IC₅₀ value for the form or formulation of the active agent.

To measure the ability of crystalline and non-crystalline forms and formulations of 1-[5-(4-amino-7-isopropyl-7H-pyrrolo[2,3-d]pyrimidine-5-carbonyl)-2-methoxy-phenyl]-3-(2,4-dichloro-phenyl)-urea to inhibit TrkA tyrosine kinase activity for the full length protein that exists in a cellular context, the porcine aortic endothelial (PAE) cells transfected with the human TrkA may be used. Cells are plated and allowed to attach to 96-well dishes in the same media (Ham's F12) with 10 percent FBS (fetal bovine serum). Test forms or formulations of the active agent, dissolved in DMSO, are serial-diluted in 96-well assay blocks with serum free media containing 0.1 percent fatty-acid free bovine serum albumin (BSA). The cells are then washed, re-fed with serum-free media with and without test compounds, and allowed to incubate for 2 hours. At the end of the 2 hours, incubation, NGF (150 ng/ml final) is added to the media for a 10-minute incubation. The cells are washed and lysed in Tris-lysis buffer (50 mM Tris, pH 7.4, 150 mM NaCl, 1 percent NP-40, 10 percent glycerol, 2 mM Na₃VO₄, 0.5 mM EDTA, complete protease inhibitor cocktail tablets without EDTA). TBS is used as a diluter solution to mix the cell lysates. The extent of phosphorylation of TrkA is measured using an ELISA assay. The black, Maxisorb 96-well plates are custom-coated with goat anti-rabbit antibody (Pierce). The Trk(C-14)sc-11 antibody (Santa Cruz) at 0.4 μg/well is bound to the plates for 2 hours in SuperBlock Blocking Buffer in TBS (Pierce). Any unbound antibody is washed off the plates prior to addition of the cell lysate. After a 2-hour incubation of the lysates with the Trk(C-14)sc-11 antibody, the TrkA associated phosphotyrosine is quantitated by development with the HRP-conjugated PY54 antibody and SuperSignal ELISA Femto substrate (Pierce). The ability of the forms and formulations of the active agent to inhibit the NGF-stimulated autophosphorylation reaction by 50 percent, relative to NGF-stimulated controls, is reported as the IC₅₀ value.

The in vitro activity of the crystalline and non-crystalline forms and formulations of 1-[5-(4-amino-7-isopropyl-7H-pyrrolo[2,3-d]pyrimidine-5-carbonyl)-2-methoxy-phenyl]-3-(2,4-dichloro-phenyl)-urea in inhibiting the TrkB receptor may be determined by the following procedure.

The ability of the compounds of the present invention to inhibit tyrosine kinase activity of TrkB may be measured using a recombinant enzyme in an assay that measures the ability of compounds to inhibit the phosphorylation of the exogenous substrate, polyGluTyr (PGT, Sigma™, 4:1). The kinase domain of the human BDNF/TrkB receptor is expressed in Sf9 insect cells as a glutathione S-transferase (GST)-fusion protein using the baculovirus expression system. The protein is purified from the lysates of these cells using glutathione agarose affinity columns. The enzyme assay is performed in 96-well plates that are coated with the PGT substrate (10 ug PGT per well). The ATP is diluted in phosphorylation buffer (50 mM HEPES, pH 7.4, 0.14M NaCl, 0.56 mM MnCl₂, 0.1 mM DTT 0.2 mM Na₃VO₄). The final concentration of ATP in the plates is 300 uM. Test compounds are first diluted in dimethylsulfoxide (DMSO) and then serial-diluted in a 96-well plate. When added to the PGT plates, the final concentration of DMSO in the assay is 0.06 percent. The recombinant enzyme is diluted in phosphorylation buffer without MnCl₂. The reaction is initiated by the addition of the recombinant enzyme to the ATP and to the test compounds. After a 2.5-hour incubation at 30° C. with shaking, the reaction is stopped with 0.5M EDTA, pH 8.0, and then aspirated. The plates are washed with wash buffer (1× imidazole wash buffer). The amount of phosphorylated PGT is quantitated by incubation with a HRP-conjugated antiphosphotyrosine antibody, developed with ABTS substrate, and the reaction is quantitated on a Wallac Victor² plate reader at 405 nm. Inhibition of the kinase enzymatic activity by the test compound is detected as a reduced absorbance, and the concentration of the compound that is required to inhibit the signal by 50 percent is reported as the IC₅₀ value for the test compound.

To measure the ability of the compounds to inhibit TrkB tyrosine kinase activity for the full-length protein that exists in a cellular context, the porcine aortic endothelial (PAE) cells transfected with the human TrkB may be used. Cells are plated and allowed to attach to 96-well dishes in the same media (Ham's F12) with 10 percent FBS (fetal bovine serum). Test compounds, dissolved in DMSO, are serial-diluted in 96-well assay blocks with serum free media containing 0.1 percent fatty-acid free bovine serum albumin (BSA). The cells are then washed, re-fed with serum free media with and without test compounds, and allowed to incubate for 2 hours. At the end of the 2-hour incubation, BDNF (100 ng/ml final) is added to the media for a 10-minute incubation. The cells are washed and lysed in Tris-lysis buffer (50 mM Tris, pH 7.4, 150 mM NaCl, 1 percent NP-40, 10 percent glycerol, 2 mM Na₃VO₄, 0.5 mM EDTA, complete protease inhibitor cocktail tablets without EDTA). TBS is used as a diluter solution to mix the cell lysates. The extent of phosphorylation of TrkB is measured using an ELISA assay. The black, Maxisorb 96-well plates are custom-coated with goat anti-rabbit antibody (Pierce). The L-Trk(C-14)sc-11 antibody (Santa Cruz) at 0.4 μg/well is bound to the plates for 2 hours in SuperBlock Blocking Buffer in TBS (Pierce). Any unbound antibody is washed off the plates prior to addition of the cell lysate. After a 2-hour incubation of the lysates with the Trk(C-14)sc-11 antibody, the TrkB associated phosphotyrosine is quantitated by development with a HRP-conjugated antiphosphotyrosine antibody and SuperSignal ELISA Femto substrate (Pierce). The ability of the compounds to inhibit the BDNF-stimulated autophosphorylation reaction by 50 percent, relative to BDNF-stimulated controls, is reported as the IC₅₀ value for the test compound.

Administration of the compound of the present invention (herein the “active compound”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

A preferred administration is oral administration of a spray dry dispersion or a pharmaceutical composition of a spray dry dispersion.

The amount of the active compound administered will be dependent on the subject being treated, the severity of the disorder or condition, the rate of administration and the judgement of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effects provided that such larger doses are first divided into several small doses for administration throughout the day.

The active compound may be applied as a sole therapy or may involve one or more other anti-tumor substances, for example those selected from, for example, mitotic inhibitors, for example vinblastine; alkylating agents, for example cisplatin, carboplatin and cyclophosphamide; anti-metabolites, for example 5-fluorouracil, cytosine arabinoside and hydroxyurea, or, for example, one of the preferred antimetabolites disclosed in European Patent Application No. 239362 such as N-(5-(N-(3,4-dihydro-2-methyl-4-oxoquinazolin-6-ylmethyl)-N-methylamino]-2-thenoyl)-L-glutamic acid; growth factor inhibitor; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-trifluoromethyl)propionanilide). Such conjoint treatment may be achieved by way of simultaneous, sequential or separate dosing of the individual components of the treatment.

The pharmaceutical composition may, for example, be in a form suitable for oral administration as a tablet, capsule, pill, powder, sustained release formulations, solution, and suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For example, see Remington's Pharmaceutical Sciences, Mack Publishing Company, Easter, Pa. 15^{th} Edition (1975).

The examples and preparations provided below further illustrate and exemplify the compounds of the present invention and methods of preparing such compounds. It is to be understood that the scope of the present invention is not limited in any way by the scope of the following examples and preparations.

Detailed analytical and preparative HPLC chromatography methods referred to in the preparations and examples below are outlined as follows.

Analytical HPLC method 1, 2 and 3: Gilson HPLC equipped with a diode array detector and a MetaChem Polaris 5 um C18-A 20×2.0 mm column; peak detection reported usually in total intensity chromatogram and 210 nm wavelength; solvent A: water with 2 percent acetonitrile and 0.01 percent formic acid, solvent B: acetonitrile with 0.05 percent formic acid; flow rate at 1 mL/minute.

Method 1 gradient: 5 percent to 20 percent solvent B in 1 minute, ramp up to 100 percent solvent B at 2.25 minutes, stay at 100 percent B until 2.5 minutes, and back to 5 percent B at 3.75 minutes.

Method 2 gradient: 5 percent to 20 percent solvent B in 1.25 minutes, ramp up to 50 percent at 2.5 minutes, and up to 100 percent B at 3.25 minutes, stay at 100 percent B until 4.25 minutes, and back to 5 percent B at 4.5 minutes.

Method 3 gradient: stay at 0 percent solvent B until 1.0 minute, ramp up to 20 percent at 2.0 minutes, up to 100 percent B at 3.5 minutes, back to 0 percent B at 3.75 minutes.

Analytical HPLC method 4: Hewlett Packard-1050 equipped with a diode array detector and a 150×4 mm Hewlet Packard ODS Hypersil column; peak detection reported at 254 and 300 nm wavelength; solvent A: water with ammonium acetate/acetic acid buffer (0.2 M), solvent B: acetonitrile; flow rate at 3 mL/minute.

Method 4 gradient: 0 percent to 100 percent B in 10 minutes, hold at 100 percent B for 1.5 minutes.

Preparative HPLC method: Shimadzu HPLC equipped with a diode array detector and a Waters Symmetry or Extera C8 column, 19×50 mm or 30×50 mm; peak detection reported usually at 210 nm wavelength solvent A: water with 2 percent acetonitrile and 0.1 percent formic acid, solvent B: acetonitrile with 0.1 percent formic acid; flow rate between 18 to 40 mL/minute.

General preparative HPLC gradient methods are usually a linear 0 to 5 percent B to 100 percent B over 10 to 25 minutes. Special gradient methods with a narrower gradient window, customized using methods familiar to those skilled in the art, are used for some compounds.

### EXAMPLE 1

### 1-[5-(4-Amino-7-isopropyl-7H-pyrrolo[2,3-d]pyrimidine-5-carbonyl)-2-methoxy-phenyl]-3-(2,4-dichloro-phenyl)-urea•Phosphate

A mixture of 1-[5-(4-Amino-7-isopropyl-7H-pyrrolo[2,3-d]pyrimidine-5-carbonyl-2-methoxy-phenyl]-3-(2,4-dichloro-phenyl)-urea (37.5 g, 73 mmol) and ethyl acetate/ethanol/dichloromethane (10/9/20, 3.9 L) in a 5 liter flask was heated over a steam bath. Additional dichloromethane (500 mL) was added during heating until the starting material was completely dissolved. The solution was cooled for 10-15 minutes and phosphoric acid (7.52 g, 1.685 density, 85 percent by weight, 76.7 mmol) was added. Within a minute solid began to precipitate and the slurry was stirred over night at room temperature. The reaction mixture was reduced to 1.5 L and filtered to yield the title compound as white solid (Polymorph A). MP 202° C. (DSC); Hygroscopicity: 0.8 percent (by weight) at 90 percent relative humidity at ambient temperature (RH); Characteristic X-ray powder diffraction peaks (2-theta, [percent relative intensity]): 4.594 [44.6], 6.222 [100]; Combustion analysis (theoretical/experimental) of monophosphate salt: carbon (47.15/47.16), hydrogen (4.12/3.79), nitrogen (13.75/13.55) chlorine (11.60/11.74), phosphorous (5.07/5.07).

### EXAMPLE 2

### 1-[5-(4-Amino-7-isopropyl-7H-pyrrolo[2,3-d]pyrimidine-5-carbonyl)-2-methoxy-phenyl]-3-(2,4-dichloro-phenyl)-urea•Mesylate (Form B)

A suspension of 1-[5-(4-Amino-7-isopropyl-7H-pyrrolo[2,3-d]pyrimidine-5-carbonyl)-2-methoxy-phenyl]-3-(2,4-dichloro-phenyl)-urea (500 mg, 0.97 mmol) and ethanol (50 mL) was heated over a steam bath. A mixture of ethanol/dichloromethane (9/1.5, 105 mL) was added until the starting material was completely dissolved. The solution was cooled to room temperature and methanesulfonic acid (98 mg, 1.02 mmol) was added dropwise. The mixture was stirred at room temperature (3 d), concentrated in vacuo, without heat, and the precipitate was filtered and stirred in ethyl acetate at 40° C. for 12 h. The solid was filtered and dried to yield the title compound as a white solid (polymorph B). MP 195° C. (DSC); Hygroscopicity: 7.9 percent (by weight) at 90 percent relative humidity at ambient temperature (RH); Characteristic X-ray powder diffraction peaks (2-theta, [percent relative intensity]): 4.594 [44.6], 6.222 [100]; Combustion analysis (theoretical/experimental) of monomesylate salt: carbon (49.27/49.08), hydrogen (4.30/4.04), nitrogen (13.79/13.49), chlorine (11.63/11.37), sulfur (5.26/5.24).

### EXAMPLE 3

### 1-[5-(4-Amino-7-isopropyl-7H-pyrrolo[2,3-d]pyrimidine-5-carbonyl)-2-methoxy-phenyl]-3-(2,4-dichloro-phenyl)-urea•Mesylate (Polymorph A)

A suspension of 1-[5-(4-Amino-7-isopropyl-7H-pyrrolo[2,3-d]pyrimidine-5-carbonyl)-2-methoxy-phenyl]-3-(2,4-dichloro-phenyl)-urea (100 mg, 0.19 mmol) in tetrahydrofuran (6.5 mL) was heated over a steam bath until the starting material was completely dissolved. The solution was cooled to room temperature and methanesulfonic acid (0.19.6 mg, 0.20 mmol) was added. The mixture was stirred at room temperature for 24 hand filtered to yield the title compound as a white solid (Polymorph A). MP 212° C. (DSC); Hygroscopicity: 1.6 percent (by weight) at 90 percent relative humidity at ambient temperature (RH); Characteristic X-ray powder diffraction peaks (2-theta, [percent relative intensity]): 6.554 [100]: Combustion analysis (theoretical/experimental) or monomesylate salt: carbon (49.27/49.37), hydrogen (4.30/4.02), nitrogen (13.79/13.68), chlorine (11.63/11.58), sulfur (5.26/5.49).

### EXAMPLE 4

### 1-[5-(4-Amino-7-isopropyl-7H-pyrrolo[2,3-d]pyrimidine-5-carbonyl)-2-methoxy-phenyl]-3-(2,4-dichloro-phenyl)-urea•Mesylate

1-[5-(4-Amino-7-isopropyl-7H-pyrrolo[2,3-d]pyrimidine-5-carbonyl)-2-methoxy-phenyl]-3-(2,4-dichloro-phenyl)-urea mesylate was added to ethyl acetate to form a slurry and was heated at 40° C. The product was filtered to yield the title compound as a crystalline white 724 mg of white solid (720 mg, Melting Point 212° C.). Analytical calculated for C₂₄NH₆O₃Cl₂•CH₄SO₃ C 49.27, H 4.30, N 13.79; Found: C 49.20, H 4.09, N 13.79 C 49.55, H 3.95, N 13.57.

### EXAMPLE 5

### 1-[5-(4-Amino-7-isopropyl-7H-pyrrolo[2,3-d]pyrimidine-5-carbonyl)-2-methoxy-phenyl]-3-(2,4-dichloro-phenyl)-urea•Mesylate

1-[5-(4-Amino-7-isopropyl-7H-pyrrolo[2,3-d]pyrimidine-5-carbonyl)-2-methoxy-phenyl]-3-(2,4-dichloro-phenyl]-urea mesylate was added to tetrahydrofuran and heated to 40° C. The product was filtered to yield the title compound as a 638 mgs of white solid (640 mg, Melting Point 196-198° C.). Analytical calculated for C₂₄HN₆O₃Cl₂•CH₄SO₃ C 49.27, H 4.30, N 13.79; Found: C 49.44, H 4.12, N 13.63, C 49.13; H 4.04; N 13.58.

### EXAMPLE 6

### 1-[5-(4-Amino-7-isopropyl-7H-pyrrolo[2,3-d]pyrimidine-5-carbonyl)-2-methoxy-phenyl]-3-(2,4-dichloro-phenyl)-urea•Mesylate (Form B)

A suspension of 1-[5-(4-Amino-7-isopropyl-7H-pyrrolo[2,3-d]pyrimidine-5-carbonyl)-2-methoxy-phenyl]-3-(2,4-dichloro-phenyl)-urea (500 mgs; 974 mmols) in ethanol (50 mL) was heated over a steam bath. A mixture of ethanol/dichloromethane 9/1.5, 105 mL) was added until the starting material was completely dissolved. The solution was cooled to room temperature and methanesulfonic acid (0.098 grams, 1.482 density, 99.5 percent by weight, 1.0 mmol) was added dropwise. The mixture was stirred at room temperature (3 d). The resulting slurry was concentrated in vacuo, without heat, and the precipitate was filtered and stirred in ethyl acetate at 40° C. for 12 hours. The solid was filtered to yield the title compound as a white solid (polymorph B).

### EXAMPLE 7

### 1-[5-(4-Amino-7-isopropyl-7H-pyrrolo[2,3-d]pyrimidine-5-carbonyl)-2-methoxy-phenyl]-3-(2,4-dichloro-phenyl)-urea•Mesylate (Polymorph A)

A suspension of 1-[5-(4-Amino-7-isopropyl-7H-pyrrolo[2,3-d]pyrimidine-5-carbonyl)-2-methoxy-phenyl]-3-(2,4-dichloro-phenyl)-urea (0.1 gms) in tetrahydrofuran (6.5 mL) was heated over a steam bath until the starting material was completely dissolved. The solution was cooled to room temperature and methanesulfonic acid (0.020 grams, 1.482 density, 0.20 mmol) was added. The mixture was stirred at room temperature for 7 days and filtered to yield the title compound as a white solid (polymorph A).

### EXAMPLE 8

### 1-[5-(4-Amino-7-isopropyl-7H-pyrrolo[2,3-d]pyrimidine-5-carbonyl)-2-methoxy-phenyl]-3-(2,4-dichloro-phenyl)-urea•Besylate

1-[5-(4-Amino-7-isopropyl-7H-pyrrolo[2,3-d]pyrimidine-5-carbonyl)-2-methoxy-phenyl]-3-(2,4-dichloro-phenyl)-urea (1.85 gm, 3.66 mmol) was dissolved in dichloromethane/ethanol (5/0.7, 57 mL). Benzene sulfonic acid (0.59 g) in dichloromethane (10 mL) was added to the reaction mixture and stirred at room temperature for 1 hour. The reaction mixture was filtered and the filtrate was slurried in ethyl acetate for 6 days. The solid was filtered and dried to yield the title compound (750 mg, 31 percent). MP 250° C. (DSC); Hygroscopicity: 2.0 percent (by weight) at 90 percent relative humidity at ambient temperature (RH); Characteristic X-ray powder diffraction peaks (2-theta, [percent relative intensity]): 4.594 [44.6], 6.222 [100]; Combustion analysis (theoretical/experimental) of monobesylate salt: carbon (53.66/53.38), hydrogen (4.20/3.94), nitrogen (12.51/12.16), chlorine (10.56/10.49), sulfur (4.77/4.60).

### EXAMPLE 9

### 1-[5-(4-Amino-7-isopropyl-7H-pyrrolo[2,3-d]pyrimidine-5-carbonyl)-2-methoxy-phenyl]-3-(2,4-dichloro-phenyl)-urea•Tosylate

1-[5-(4-Amino-7-isopropyl-7H-pyrrolo[2,3-d]pyrimidine-5-carbonyl)-2-methoxy-phenyl]-3-(2,4-dichloro-phenyl)-urea (850 mg, 1.66 mmol), p-toluene sulfonic acid (303 mg, 1.74 mmol), dichloromethane (5.0 mL) and ethyl acetate (20 mL) were stirred at room temperature for 30 minutes. The mixture was partially concentrated in vacuo stirred for an additional 2 hours. The reaction mixture was filtered and the white solid was stirred in ethyl acetate for 5 d, the slurry was filtered and dried to yield the title compound as a white solid (0.82 g, 72 percent). Melting point 280-282° C. Analytical calculated for C₂₄HN₆O₃Cl₂•C₇H₉SO₃ C 54.31, H 4.41, N 12.26; Found; C 54.07, H 4.10, N 12.10; C 54.04, H 4.13, N 12.05. MP 255° C. (DSC); Hygrosopicity: 1.1 percent (by weight) at 90 percent relative humidity at ambient temperature (RH); Characteristic X-ray powder diffraction peaks (2-theta, [percent relative intensity]): 4.594 [44.6], 6.222 [100]; Combustion analysis (theoretical/experimental) of monotosylate salt: carbon (54.31/53.89), hydrogen (4.41/4.19), nitrogen (12.26/12.08), chlorine (10.34/10.46), sulfur (4.68/4.58).

### EXAMPLE 10

### 1-[5-(4-Amino-7-isopropyl-7H-pyrrolo[2,3-d]pyrimidine-5-carbonyl)-2-methoxy-phenyl]-3-(2,4-dichloro-phenyl)-urea

Step 1.: 4-chloro-7-isopropyl-7H-pyrrolo[2,3-d]pyrimidine

4-Chloro-7H-pyrrolo[2,3-d]pyrimidine (7.5 kg; 1 equivalent) was dissolved in 2-methylpyrrolidinone (30.8 L, 4 volumes). Cesium carbonate (31.8 kg, 2 equivalents), dimethylformamide (3.7 L, 0.5 volumes) and 2-iodopropane (16.7 kg, 2 equivalents) were then added to the reactor, keeping the temperature between 20 and 30° C. The reaction was maintained at 20-25° C. for 5 hours. Ethyl acetate (60 L, 7 volumes) was added and the resulting slurry filtered through Celite™ to remove insolubles. The organic layer was extracted three times with brine (60 L, 4 volumes each) and then decolorized with Darco KBB (1.5 kg, 20 wt percent). Water (60 L, 4 volumes) was added to the filtrate, the ethyl acetate was removed by distillation, and the resulting slurry was granulated in water, isolated, and dried under vacuum.

Step 2.: 5-bromo-4-chloro-7-isopropyl-7H-pyrrolo[2,3-d]pyrimidine

The filtrate (4-chloro-7-isopropyl-7H-pyrrolo[2,3-d]pyrimidine (7.0 kg, 1 equivalent)) was dissolved in acetone (35 L, 5 volumes). In a separate vessel, N-bromosuccinimide (6.4 kg, 1 equivalent) was dissolved in acetone (70 L, 10 volumes) and was then transferred over approximately 90 minutes to the solution of 4-chloro-7-isopropyl-7H-pyrrolo[2,3-d]pyrimidine keeping the temperature between 15-25° C. The reaction was held at 15-25° C. for an additional 30 minutes and then the volume of the reaction was reduced to 35 L (5 volumes) by vacuum distillation. A solution of sodium thiosulfate pentahydrate (23.3 kg, approximately 2.75 equivalents) in water (35 L, 5 volumes) was added to produce a biphasic system. The phases were mixed for 30 minutes and then separated. The reaction mixture was then cooled to about 10° C. and water (45 L, 6.7 volumes) was added to crystallize 5-bromo-4-chloro-7-isopropyl-7H-pyrrolo[2,3-d]pyrimidine, which was isolated by filtration, washed with water, and dried.

Step 3.: 4-chloro-7isopropyl-7H-pyrrolo[2,3-d]pyrimidin-5-yl(4-methoxy-3-nitrophenyl)-methanone

1 equivalent (7.1 kg) of 5-bromo-4-chloro-7-isopropyl-7H-pyrrolo[2,3-d]pyrimidine was first dissolved in 35 L (5 volumes) of toluene. This solution was then cooled down to −20° C. 1.05 equivalent of n-butyllithium (10.8 L, 2.5M in hexanes) was then added slowly over about an hour to minimize any exotherm. Upon completion of n-butyllithium addition, the reaction was cooled to between −70° C. and −80° C. 1.2 equivalent of 4-methoxy-3-nitrobenzoyl chloride (7.5 kg) was dissolved in 5 volume of toluene and this solution was added slowly to the cooled solution of lithiated 5-bromo-4-chloro-7-isopropyl-7H-pyrrolo[2,3-d]pyrimidine. Addition rate (over 15 minutes) was maintained so that the temperature in the reactor was maintained below −70° C. The reaction was allowed to proceed at −78° C. for 8 hours at which point a light yellow slurry was formed. The reaction was allowed to warm slowly to ambient temperature. Water (35 L, 5 volumes) was added to quench the reaction, and the product was obtained by filtration.

Step 4.: 4-amino-7-isopropyl-7H-pyrrolo[2,3-d]pyrimidin-5-yl)(4-methoxy-3-nitrophenyl) methanone

The filtrate ((4-chloro-7-isopropyl-7H-pyrrolo[2,3-d]pyrimidin-5-yl)(4-methoxy-3-nitrophenyl)methanone; 4.9 kg) was mixed in with 15 L (3 volumes) of concentrated ammonium hydroxide and 15 L (3 volumes) of tetrahydrofuran. The reaction mixture was then sealed up in a pressure reactor and heated to between 50° C. and 60° C. for around 24 hours. (The maximum pressure was approximately 30 psi.) The reaction was then quenched into 50 L (10 volumes of water, and the product was obtained by filtration.

Step 5.: 3-amino-4-methoxyphenyl)(4-amino-7-isopropyl-7H-pyrrolo[2,3-d]pyrimidin-5-yl)methanone

The product ((4-amino-7-isopropyl-7H-pyrrolo[2,3-d]pyrimidine-5-yl)(4-methoxy-3-nitrophenyl) methanone; 3.9 kg) was mixed with 5 percent palladium on carbon (390 g 10 percent by weight) dissolved in 40 L (10 volumes) of tetrahydrofuran. The reaction mixture was then warmed to between 40° C. and 50° C., 45 psig. The reaction continued for approximately 36 to 48 hours. Upon completion of hydrogenation, the mixture was filtered and 3-amino-4-methoxyphenyl)(4-amino-7-isopropyl-7H-pyrrolo[2,3-d]pyrimidin-5-yl)methanone was isolated by displacing tetrahydrofuran with toluene under vacuum.

Step 6.: 1-[5-Amino-7-isopropyl-7H-pyrrolo[2,3-d]pyrimidine-5-carbonyl)-2-methoxy-phenyl]-3-(2,4-dichloro-phenyl)-urea

1 equivalent of the product ((3-amino-4-methoxyphenyl)(4-amino-7-isopropyl-7H-pyrrolo[2,3-d]pyrimidin-5-yl)methanone; 3.1 kg) was mixed in with 3.1 L (1 volume) of pyridine and 12.4 L (4 volumes) of ethyl acetate. The reaction mixture was cooled to between 5° C. and 10° C. 1.01 equivalent (1.84 kg) of 2,4-dichloro-1-isocyanatobenzene (solid) was then added in positions to control exotherm.

Upon completion of addition, HPLC assay showed possibly some starting material remained. Additional 0.1 (184 g) equivalents of 2,4-dichloro-1-isocyanatobenzene was added. Product was then obtained by filtration.

The product (1-[5-(4-Amino-7-isopropyl-7H-pyrrolo[2,3-d]pyrimidine-5-carbonyl)-2-methoxy-phenyl]-3-(2,4-dichloro-phenyl)-urea) was dissolved in 35 L (10 volumes) of pyridine and then filtered. The solution was then charged to 70 L (20 volumes) of filtered water and 1-[5-(4-Amino-7-isopropyl-7H-pyrrolo[2,3-d]pyrimidine-5-carbonyl)-2-methoxy-phenyl]-3-(2,4-dichloro-phenyl)-urea precipitated, and the solid was isolated by filtration and washed with 7 L (2 volumes) of filtered water.

Alternatively, 1-[5-(4-Amino-7-isopropyl-7H-pyrrolo[2,3-d]pyrimidine-5-carbonyl)-2-methoxy-phenyl]-3-(2,4-dichloro-phenyl)-urea was mixed in a combination of 17.5 L (5 volumes) of ethanol and 3.5 L (1 volume) of acetone. The slurry was stirred at between 25° C. and 30° C. for around 48 hours. Product was then obtained by filtration, washed with 7 L (2 volumes) of ethanol. Isolated solid was dried in vacuum oven between 50° C. and 60° C.

3.2 kg or product was obtained.

### EXAMPLE 11

### 1-[5-(4-Amino-7-isopropyl-7H-pyrrolo[2,3-d]pyrimidine-5-carbonyl)-2-methoxy-phenyl]-3-(2,4-dichloro-phenyl)-urea

Step 1: 4-chloro-7H-pyrrolo[2,3-d]pyrimidine

30.5 kg of N,N-diisopropylethylamine (Hunig's base; 1.1 equiv.) and 29 Kg 7H-pyrrolo[2,3-d]pyrimidin-4-ol (1 equiv.) was added to 145 L of toluene (5 L/Kg), an 65.8 Kg phosphoryl trichloride (2.0 equiv.) at a rate so that the temperature did not exceed 30° C. The reaction was heated to 115° C. for a minimum of 3 hours and then cooled to 25° C. and transferred to a solution of 435 L (15 L/Kg):29 L (1 L/Kg) water:tetrahydrofuran (THF). During transfer, the reaction temperature did not exceed 50° C. 50 percent NaOH (36 Kg 4.2 equiv.) was then added at a rate so that the temperature did not exceed 40° C. and the reaction was stirred for 1 hour. The reaction was concentrated to 145 L (5 L/Kg), and the resulting 4-chloro-7H-pyrrolo[2,3-d]pyrimidine was granulated for 2 hours, filtered and washed with water (116 L, 4 L/Kg).

Step 2: 5-bromo-4-chloro-7-isopropyl-7H-pyrrolo[2,3-d]

29.5 Kg of 4-chloro-7H-pyrrolo[2,3-d]pyrimidine (1 equiv.) was added to 30 LDMF (1 L/Kg) and 450 L of 2-methyltetrahydrofuran (2-MeTHF; 15 L/Kg), 2-iodopropane (49 kg, 1.5 equiv.) and cesium carbonate (94 kg, 1.5 equiv.). The reaction was heated to 80° C. for 3 hours. Ethyl acetate (148 L, 5 L/Kg) was then added and cesium carbonate was filtered away from reaction and rinsed with ethyl acetate (236 L, 8 L/Kg). The mother liquor was washed with 1 N HCl (295 L, 10 L/Kg) and brine (295 L, 10 L/Kg), and then concentrated to remove the ethyl acetate. The solution was then added to a slurry of N-Bromosuccinimide (NBS; 34 kg, 1.9 equiv.) in 2-MeTHF (295 L, 10 L/Kg) at a rate such that the temperature did not exceed 30° C. The reaction was stirred for 1 hour and quenched with saturated sodium thiosulfate (295 L, 10 L/Kg)). After separation of the organic from aqueous layer, water (295 L, 10 L/Kg) was added and the 2-MeTHF was concentrated off. The resulting 5-bromo-4-chloro-7-isopropyl-7H-pyrrolo[2,3-d]pyrimidine was granulated in water, filtered and dried.

Step 3: 4-chloro-7-isopropyl-7H-pyrrolo[2,3-d]pyrimidin-5-yl)(4-methoxy-3-nitrophenyl)methanone

5-Bromo-4-chloro-7-isopropyl-7H-pyrrolo[2,3-d]pyrimidine CE-265,894 (1 equiv.) was added to toluene (10 L/Kg) and the reaction was cooled to 0° C. or −20° C. and 2.5 M n-butyl lithium (n-BuLi (1.1 equiv.)) was added at a rate so that the temperature did not go above 5° C. or −18° C. The reaction was stirred for 1 hour and quenched with N,4-dimethoxy-N-methyl-3-nitrobenzamide (Weinreb amide; 1 equiv.) in toluene (10 L/Kg) at a rate so that the temperature did not exceed 10° C. or −10° C., (N,4-dimethoxy-N-methyl-3-nitrobenzamide was made by slowly adding 32 kg triethylamine (TEA; 2.0 equiv.) to 34 Kg 4-methoxy-3-nitrobenzoyl chloride (1 equiv.), 340 L methylene chloride (10 L/Kg) and 30.7 Kg N-methoxymethylamine•HCl (2.0 equiv.) so that the temperature did not exceed 30° C. The reaction was stirred for 3 hours and then washed with water (340 L, 10 L/Kg), saturated sodium bicarbonate (340 L, 10 L/Kg), saturated ammonium chloride (340 L, 10 L/Kg) and brine (340 L, 10 L/Kg). The product was crystallized from IPE, filtered, washed with IPE (170 L, 5 L/Kg) and dried.) The reaction was stirred for 1 hour at 5°-15° C. and quenched with water (10 L/Kg). The slurry was granulated, filtered, washed with water (5 L/Kg), and dried. Alternatively, 2 M isopropylmagnesiusm chloride (iPrMgCl; 1.4 equiv.) was added to 5-bromo-4-chloro-7-isopropyl-7H-pyrrolo[2,3-d]pyrimidine CE-265,894 (1 equiv.) in toluene (10 L/Kg) at a rate so that the temperature did not exceed 30° C. The reactor was stirred for 3 hours and at anion formation completion was quenched with Weinreb amide N,4-dimethoxy-N-methyl-3-nitrobenzamide PF-419,852 (1 equiv.) in toluene (10 L/Kg) at a rate so that the temperature did not go above 30° C. The reaction was stirred for 3 hours and quenched with water (10 L/Kg). The slurry was granulated, filtered, washed with water (5 L/Kg) and dried.

Step 4: (4-Amino-7-isopropyl-7H-pyrrolo[2,3-d]pyrimidin-5-yl)(4-methoxy-3-nitrophenyl)methanone

8.7 Kg of 4-chloro-7-isopropyl-7H -pyrrolo[2,3-d]pyrimidin-5-yl)(4-methoxy-3-nitrophenyl)methanone (1 equiv.), was added to 26-44 L of 28 percent ammonium hydroxide (3-5 L/Kg) and 26-44 LTHF (26-44 L, 3-5 L/Kg). The reaction was heated to 50° C.-60° C., and then 87 L of water (10 L/Kg) was added and the product (4-amino-7-isopropyl-7H-pyrrolo[2,3-d]pyrimidin-5-yl)(4-methoxy-3-nitrophenyl)methanone) was granulated, filtered, washed with water and dried.

Step 5: (3-amino-4-methoxyphenyl)(4-amino-7-isopropyl-7H-pyrrolo[2,3-d]pyrimidin-5-yl)methanone

6.9 Kg of (4-amino-7-isopropyl-7H-pyrrolo[2,3-d]pyrimidin-5-yl)(4-methoxy-3-nitrophenyl)methanone PF-2,373,207 (6.9 kg, 1 equiv.) and 0.69 Kg of 10 percent palladium on carton (Pd/C; 10 percent by weight) was added to 138 L of THF (20 L/Kg). The reaction was heated to 40° C. for 2 hours and then to 60° C. The catalyst was filtered and rinsed with 21 L of THF (3 L/Kg). The THF was displaced with 69 L of toluene (10 L/Kg) and the product was granulated, filtered, washed with toluene, and dried.

Step 6: 1-[5-(4-Amino-7-isopropyl-7H-pyrrolo[2,3-d]pyrimidine-5-carbonyl)-2-methoxy-phenyl]-3-(2,4-dichloro-phenyl)-urea

4.8 Kg of (3-Amino-4-methoxyphenyl)(4-amino-7-isopropyl-7H-pyrrolo[2,3-d]pyrimidin-5-yl)methanone (1 equiv.) was added to 48 L of pyridine (10 L/Kg). The reaction was stirred and 2.8 Kg of 2,4-dichloro-1-isocyanatobenzene (1 equiv.) was added in seven portions so that the temperature did not exceed 25° C. The product, 1-[5-(4-Amino-7-isopropyl-7H-pyrrolo[2,3-d]pyrimidine-5-carbonyl)-2-methoxy-phenyl]-3-(2,4-dichloro-phenyl)-urea, was quenched into water (96 L, 20 L/Kg), and the slurry was stirred for 8 hours. The material was filtered and the polymorph was converted with a 40° C. water repulp (48 L, 10 L/Kg). The material was filtered again and then repulped with THF (48 L, 10 L/Kg). The material was filtered, washed with THF (24 L, 5 L/Kg) and dried. The final amount of material isolated was 3.9 kg (overall process yield was 3.5 percent).

### EXAMPLE 12

Step 1: 4-chloro-7H-pyrrolo[2,3-d]pyrimidine:

18.5 Kg of 7H-pyrrolo[2,3-d]pyrimidin-4-ol (1 equivalentsalent) and 42 Kg POCl₃ (2.0 equivalents) were added to 93 L toluene (5 L/Kg), N,N-diisopropylethylamine (Hunig's base; 19.4 kg, 1.1 equivalents) was then added at a rate so that the temperature did not exceed 30° C. The reaction was heated to reflux (˜115° C.) for a minimum of 3 hours and then cooled to 25° C. and transferred to a solution of 278 L (15 L/Kg):19 L (1 L/Kg) water:tetrahydrofuran (THF). During transfer, the reaction temperature did not exceed 50° C. 50 percent NaOH (23 kg, 4.2 equivalents or to pH 7) was then added at a rate so that the temperature did not exceed 40° C. and the reaction was stirred for 1 hour. The solution was then concentrated and 4-chloro-7H-pyrrolo[2,3-d]pyrimidine was granulated for 2 hours, filtered and washed with water (74 L, 4 L/Kg). The material was repulped in water (185 L, 10 L/Kg).

Step 2: 5-bromo-4-chloro-7-isopropyl-7H-pyrrolo[2,3-d]pyrimidine:

15 Kg 4-chloro-7H-pyrrolo[2,3-d]pyrimidine (1 equivalent) was added to 30 L dimethylformamide (DMF; 2 L/Kg), 150 L 2-methyltetrahydrofuran (2-MeTHF; 10 L/Kg), 25 Kg 2-iodopropane (1.5 equivalents) and 48 Kg cesium carbonate (1.5 equivalents). The reaction was heated to reflux (80° C.) for 3 hours before 300 L ethyl acetate (20 L/Kg) was added and cesium carbonate was filtered away from the reaction and rinsed with ethyl acetate (120 L, 8 L/Kg). The mother liquor was washed with 1 N HCl (150 L, 10 L/Kg) and brine (150 L, 10 L/Kg), and then concentrated to remove ethyl acetate. The solution was further displaced into 2-MeTHF (75 L, 5 L/Kg), and then added to a slurry of N-Bromosuccinimide (NBS; 21 kg, 1.2 equivalents) in 2-MeTHF (150 L, 10 L/Kg) at a rate such that the temperature did not exceed 30° C. The reaction was stirred for 1 hour before an additional 12.2 Kg NBS (0.7 equivalents) was added. The reaction was quenched with saturated sodium thiosulfate (150 L, 10 L/Kg) and then 150 L water (10 L/Kg) was added. After separation of the organic from the aqueous layer, water (150 L, 10 L/Kg) was added and the 2-MeTHF was concentrated off. The material was diluted with ethyl acetate (150 L, 10 L/Kg), the layers separated, and then the material was darco treated. The product, 5-bromo-4-chloro-7-isopropyl-7H-pyrrolo[2,3-d]pyrimidine, was concentrated again and displaced into heptanes (75 L, 5 L/Kg), filtered and dried.

Step 3: (4-chloro-7-isopropyl-7H-pyrrolo[2,3-d]pyrimidin-5-yl)(4-methoxy-3-nitrophenyl)methanone:

16.8 Kg 5-bromo-4-chloro-7-isopropyl-7H-pyrrolo[2,3-d]pyrimidine (1 equivalent) was added to 135 L THF (8 L/Kg), 171 L 2 M isopropylmagnesium chloride (iPrMgCl; 1.4 equivalents) was added at a rate so that the temperature did not exceed 35° C. The reaction was stirred for 3 hours and then quenched with N,4-dimethoxy-N-methyl-3-nitrobenzamide (Weinreb amide; 15 kg, 1 equivalent) in toluene (168 L, 10 L/Kg) at a rate so that the temperature did not exceed 30° C. [N,4-dimethoxy-N-methyl-3-nitrobenzamide was synthesized by adding 25 Kg 4-methoxy-3-nitrobenzoic acid (1.0 equivalent) and 22.6 Kg N,N′-carbonyldiimidazole (CDI; 1.2 equivalents) to 250 L dichloromethane (10 L/Kg). The reaction was held at 20° C.-30° C. for a minimum of 3 hours and then N-methoxymethylamine hydrochloride (17 kg, 1.5 equivalents) was added. The reaction was cooled to 15° C. and triethylamine (TFA; 16.4 kg, 1.4 equivalents) was added at a rate such that the temperature did not exceed 25° C. The reaction was stirred for a minimum of 3 hours and quenched into water (250 L, 10 L/Kg). The resulting product, N,4-dimethoxy-N-methyl-3-nitrobenzamide, was washed with 1 N HCl (250 L, 10 L/kg) and then sodium bicarbonate (250 L, 10 L/Kg). The material was displaced into IPE (250 L, 10 L/Kg) and concentrated to 100 L (4 L/Kg). N,4-dimethoxy-N-methyl-3-nitrobenzamide was granulated for a minimum of 3 hours, filtered and dried. The reaction was stirred for 4 hours and quenched with water (168 L, 10 L/Kg). The reaction was pH'd to 5-7 with concentrated HCl (2.95 Kg, 1.34 equivalents) and concentrated until the distillation stopped. (4-chloro-7-isopropyl-7H-pyrrolo[2,3-d]pyrimidin-5-yl)(4-methoxy-3-nitrophenyl)methanone was granulated, filtered, washed with IPO (4 L, 0.22 L/Kg) and dried.

Step 4: (4-amino-7-isopropyl-7H-pyrrolo[2,3-d]pyrimidin-5-yl)(4-methoxy-3-nitrophenyl)methanone:

15.9 Kg (4-chloro-7-isopropyl-7H-pyrrolo[2,3-d]pyrimidin-5-yl)(4-methoxy-3-nitrophenyl)methanone (1 equivalents) was added to 48 L 28 percent ammonium hydroxide (3 L/Kg) and 80 L THF (5 L/Kg). The reaction was heated to 50° C.-55° C. and then 159 L of water (10 L/Kg) was added and (4-amino-7-isopropyl-7H-pyrrolo[2,3-d]pyrimidin-5-yl)(4-methoxy-3-nitrophenyl)methanone was granulated, filtered, washed with water (80 L, 5 L/Kg) and dried. A reslurry was performed in THF (32-48 L, 2-3 L/Kg) to remove impurities.

Step 5: (3-amino-4-methoxyphenyl)(4-amino-7-isopropyl-7H-pyrrolo[2,3-d]pyrimidin-5-yl)methanone:

4.5 Kg (4-amino-7-isopropyl-7H-pyrrolo[2,3-d]pyrimidin-5-yl)(4-methoxy-3-nitrophenyl)methanone (1 equivalent) and 0.45 Kg 10 percent palladium on carbon (Pd/C; 10 percent by weight) was added to 90 L THF (20 L/Kg). The reaction was heated to 40° C. for 2 hours and then to 60° C. The catalyst was filtered off and rinsed with THF (90 L, 20 L/Kg). The THF was displaced with toluene (45 L, 10 L/Kg) and (3-amino-4-methoxyphenyl)(4-amino-7-isopropyl-7H-pyrrolo[2,3-d]pyrimidin-5-yl)methanone was granulated, filtered, washed with toluene (23 L, 5 L/Kg) and dried.

Step 6: 1-[5-(4-Amino-7-isopropyl-7H-pyrrolo[2,3-d]pyrimidine-5-carbonyl)-2-methoxy-phenyl]-3-(2,4-dichloro-phenyl)-urea:

4.7 Kg (3-amino-4-methoxyphenyl)(4-amino-7-isopropyl-7H-pyrrolo[2,3-d]pyrimidin-5-yl)methanone (1 equivalent) was added to 47 ml dry pyridine (10 L/Kg). The reaction was stirred and 2.7 Kg 2,4-dichloro-1-isocyanatobenzene (1 equivalent) was added in seven portions so that the temperature remained around 28° C. The reaction was stirred at 28° C. for 1 hour and quenched into water (94 L, 20 L/Kg). The slurry was stirred for 8 hours. The material was filtered and dried, and then THF repulped (47 L, 10 L/Kg, stir for 2 hours, concentrate to 14 L, 3 L/Kg at 40° C., stir for 2 hours). 1-[5-(4-Amino-7-isopropyl-7H-pyrrolo[2,3-d]pyrimidine-5-carbonyl)-2-methoxy-phenyl]-3-(2,4-dichloro-phenyl)-urea was filtered, washed with 10° C. THF (24 L, 5 L/Kg) and dried. The final amount of material isolated was 6.2 kg.

### EXAMPLE 13

### 1-[5-(4-Amino-7-isopropyl-7H-pyrrolo[2,3-d]pyrimidine-5-carbonyl)-2-methoxy-phenyl]-3-(2,4-dichloro-phenyl)-urea

The following process was used to form a spray-dried dispersion containing 25 wt percent substance and 75 wt percent HPMCAS-HG. First, a 25,600 g spray solution was formed containing 2 wt percent 1-[5-(4-amino-7-isopropyl-7H-pyrrolo[2,3-d]pyrimidine-5-carbonyl)-2-methoxy-phenyl]-3-(2,4-dichloro-phenyl)-urea, 6 wt percent HPMCAS-HG polymer, and 92 wt percent tetrahydrofuran (technical grade) as follows. The 1-[5-(4-amino-7-isopropyl-7H-pyrrolo[2,3-d]pyrimidine-5-carbonyl)-2-methoxy-phenyl]-3-(2,4-dichloro-phenyl)-urea and tetrahydrofuran were combined in a container and mixed for at least 1 hour, allowing the 1-[5-(4-amino-7-isopropyl-7H-pyrrolo[2,3-d]pyrimidine-5-carbonyl)-2-methoxy-phenyl]-3-(2,4-dichloro-phenyl)-urea to dissolve. Next, HPMCAS-HG was added directly to this mixture, and the mixture stirred for a minimum of 2 hours. The resulting mixture had a slight haze after the entire amount of polymer had been added due to the granular form of HPMCAS containing some insoluble material (about 700 μm in size, <0.1 wt percent of polymer). This mixture was then passed through an in-line filter between the spray-solution tank and the spray nozzle to prevent obstruction of the pressure nozzle orifice, thus forming the spray solution.

The spray-dried dispersion was then formed using the following procedure. The spray solution as pumped using a high-pressure pump (a Bran Luebbe, model N-P31) to a spray drier (a Niro type XP Portable Spray-Dryer with a Liquid-Feed Process Vessel) (“PSD-1”), equipped with a pressure nozzle (Schlick 3.0), The PSD-1 was equipped with 9-inch and 4-inch chamber extensions. The spray drier was also equipped with a DPH gas disperser for introduction of the drying gas to the spray drying chamber. The DPH gas disperser minimizes hot surfaces to which the SDD particles might be exposed, minimizing SDD sticking and melting during spray-drying. The DPH lid cooling water was utilized to further minimize risk of buildup due to thermal sticking at the gas inlet. The spray solution was pumped to the spray drier at about 150 g/minute at a pressure of about 750 psi. Drying gas (e.g., nitrogen) was introduced to the spray drier through the DPH lid at an inlet temperature of about 120±10° C. The evaporated solvent and wet drying gas exited the spray drier at a temperature of 60±5° C.

The spray-dried dispersion formed by this process was collected in a cyclone mounted on the outlet duct from the drying chamber, and had a bulk specific volume 5.7 cm³/gm, with a mean particle diameter of 17 μm. Consistent hammering (at least every 10 minutes) of the drying chamber was performed to minimize the accumulation of dry powder in the spray dryer.

The dispersion formed using the above procedure was post-dried using a Gruenberg convection tray dryer with a powder depth of about 1 cm operating at 40° C. for a minimum of 8 hours. Following drying, the dispersion was equilibrated with ambient air and humidity (e.g., 20° C./50 percent RH).

Typical properties of the dispersion after secondary drying were as follows.

TABLE 1
Physical Properties Of 25 percent 1-[5-(4-amino-7-isopropyl-7H-
pyrrolo[2,3-d]pyrimidine-5-carbonyl)-2-methoxy-phenyl]-3-(2,4-
dichloro-phenyl)-urea; HPMCAS-HG Spray Dry Dispersion

| Parameter | Value |
|---|---|
| Morphology | Collapsed and |
| | uncollapsed |
| | spheres |
| Mean particle diameter (μm) | 17 |
| DV₁₀, DV₅₀, DV₉₀ (μm) | 5, 15, 31 |
| Span (D₉₀-D₁₀)/D₅₀ | 1.74 |
| Bulk specific volume (cc/g) | 5.7 |
| Tapped specific volume (cc/g) | 3.2 |
| Hausner ratio | 1.78 |
| Glass-transition temperature (T_{g})(° C.) | 116 |
| Crystallinity | No crystals |
| | detected by |
| | PXRD or SEM |
| Water content (wt percent) | 1.9 |

In the Table above, DV₁₀ means that vol % of the particles had a diameter that was smaller than D₁₀; DV₅₀ means that vol % of the particles had a diameter that was smaller than D₅₀, and DV₉₀ means that vol % of the particles had a diameter that was smaller than D₉₀.

### TIE-2 Metabolites

Administration of active agent to dog and rat resulted in identification of certain metabolites that are identified in the Table below. The present invention relates to each of these individual metabolites.

| | |
|---|---|
| C₁₇H₁₉N₅O₂HRMS 325.37 | 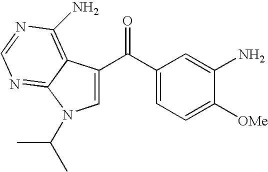 |
| C₃₁H₂₅Cl₄N₇O₄HRMS 699.07 | 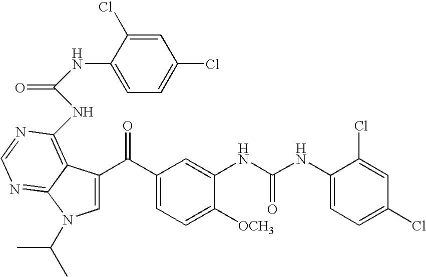 |
| C₁₃H₈Cl₄N₂O 1,3-bis(2,4- dichlorophenyl)urea HRMS 350.03 | 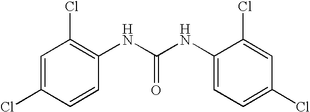 |
| C₂₃H₂₀Cl₂N₆O₃HRMS 499.35 | 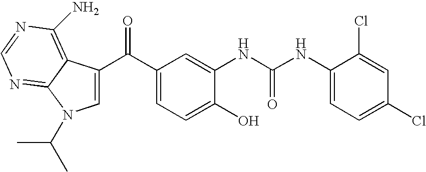 |
| C₂₃H₂₁Cl₂N₇O₂HRMS 497.11 | 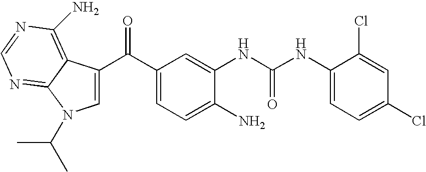 |
| C₂₃H₂₁Cl₂N₇O₂HRMS 497.11 | 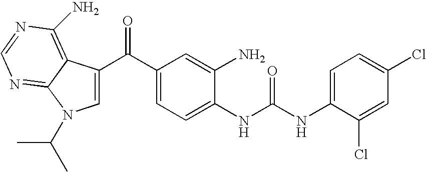 |

### Method for Collecting Powder X-ray Diffraction for 1-[5-(4-amino-7-isopropyl-7H-pyrrolo[2,3-d]pyrimidine-5-carbonyl)-2-methoxy-phenyl]-3-(2,4-dichloro-phenyl)-urea salts

Powder x-ray diffraction patterns were collected using a Bruker D5000 diffractometer (Madison, Wis.) equipped with a copper radiation source, fixed slits (1.0 mm, 1.0 mm, and 0.6 mm) and a Kevex solid-state detector. Data was collected in the theta-theta, goniometer configuration from a flat plate sample holder at the Copper wavelength Kα₁=1.54056 and Kα₂=1.54439 from 3.0 to 40.0 degrees two-theta using a step size of 0.040 degrees and a step time of one second. The results are summarized in the following tables.

TABLE 1
List of powder x-ray diffraction reflections for the
phosphate salt Form A

| Angle | Intensity |
|---|---|
| 2-Theta ± 0.2° | Percent |
| 5.3° | 100 |
| 6.4 | 24.5 |
| 7.6 | 19.6 |
| 9.0° | 31 |
| 10.5 | 18.2 |
| 12.8 | 54.4 |
| 14.2 | 17.6 |
| 15.9° | 45.1 |
| 16.6 | 14.3 |
| 18.2 | 28.2 |
| 19.5 | 19.3 |
| 21.4 | 10.6 |
| 23.2 | 40 |
| 24.1 | 23 |
| 25.1 | 10.9 |
| 25.6 | 21.9 |
| 28.9 | 27.8 |

Reflections having the greatest relative intensity at 5.3, 9.0, 12.8, 15.9, and 23.2 degrees two-theta for the phosphate salt Form A. Superscript (u) denotes unique reflections of Form A.

TABLE 2
List of powder x-ray diffraction reflections for the phosphate
salt Form B

| Angle | Intensity |
|---|---|
| 2-Theta ± 0.2° | Percent |
| 4.5° | 100 |
| 5.6 | 2.2 |
| 6.5 | 1.9 |
| 9.7 | 21.8 |
| 11.5° | 4.7 |
| 12.9 | 3.1 |
| 13.5° | 6.7 |
| 18.0 | 14.7 |
| 18.4 | 1.8 |
| 19.2 | 1.4 |
| 19.5 | 1.7 |
| 20.0 | 1.8 |
| 20.4 | 1.4 |
| 21.5 | 1.2 |
| 21.7 | 1.3 |
| 22.6 | 2.4 |
| 23.4 | 3.6 |
| 24.3 | 2.1 |
| 25.2 | 2.6 |
| 25.6 | 3.3 |
| 26.2 | 2.8 |
| 27.2 | 6.5 |
| 27.6 | 2.3 |
| 28.3 | 1.6 |
| 28.8 | 9.1 |
| 31.4 | 1.4 |

Reflections having the greatest relative intensity at 4.5, 9.7, 13.5, 18.0, and 28.8 degrees two-theta for the phosphate salt Form B. Superscript (u) denotes unique reflections of Form B.

TABLE 3
List of powder x-ray diffraction reflections for the mesylate
salt Form A

| Angle | Intensity |
|---|---|
| 2-Theta ± 0.2° | Percent |
| 6.6° | 100 |
| 8.0 | 4.3 |
| 10.0 | 17.1 |
| 12.5 | 11.7 |
| 13.2 | 5.2 |
| 13.4 | 4.2 |
| 14.1 | 5.1 |
| 15.4° | 18 |
| 16.0 | 20.4 |
| 17.0 | 11.7 |
| 17.3 | 10.5 |
| 18.0° | 13.1 |
| 18.5 | 8.8 |
| 19.4 | 10.7 |
| 20.4 | 12.6 |
| 21.2 | 6.3 |
| 21.6 | 9.7 |
| 25.1 | 15.5 |
| 25.7 | 16.4 |
| 27.3 | 6.7 |
| 28.1 | 7.5 |
| 28.6 | 11.7 |

Reflections having the greatest relative intensity at 6.6, 10.0, 12.5, 15.4, and 16.0 degrees two-theta for the mesylate salt Form A. Superscript (u) denotes unique reflections of Form A.

TABLE 4
List of powder x-ray diffraction reflections for the mesylate
salt From B

| Angle | Intensity |
|---|---|
| 2-Theta ± 0.2° | Percent |
| 4.6° | 44.6 |
| 6.2° | 100 |
| 10.1 | 4.4 |
| 12.5 | 12 |
| 14.2 | 11.6 |
| 16.0 | 22 |
| 18.5 | 3.5 |
| 19.2 | 3.7 |
| 20.6 | 3.9 |
| 21.4 | 4 |
| 22.2° | 8.3 |
| 23.2 | 14.9 |
| 24.6 | 4 |
| 25.1 | 5.9 |
| 26.0 | 7.5 |
| 27.7 | 4.1 |
| 28.8 | 4.5 |

Reflections having the greatest relative intensity at 4.6, 6.2, 12.5, 14.2, and 23.2 degrees two-theta for the mesylate salt Form B. Superscript (u) denotes unique reflections of Form B.

TABLE 5
List of powder x-ray diffraction reflections for the mesylate
salt Form C

| Angle | Intensity |
|---|---|
| 2-Theta ± 0.2° | Percent |
| 7.1° | 34.9 |
| 8.0° | 75.8 |
| 9.3 | 12.6 |
| 10.5 | 63.8 |
| 11.7 | 32.3 |
| 12.0 | 19.6 |
| 14.3 | 12.5 |
| 16.0 | 89.9 |
| 16.6 | 16.1 |
| 17.1 | 28 |
| 17.3 | 28.6 |
| 18.6 | 43.5 |
| 19.3 | 21.8 |
| 19.6° | 41.5 |
| 20.4 | 32.8 |
| 20.8 | 33.9 |
| 21.2 | 55.3 |
| 21.5 | 100 |
| 21.8 | 27.6 |
| 23.0 | 17.3 |
| 23.5 | 74.5 |
| 24.3 | 27.9 |
| 24.8 | 67.2 |
| 25.0 | 67.6 |
| 25.7 | 26.7 |
| 26.5 | 17.7 |
| 27.2 | 28.3 |
| 27.9 | 17.3 |
| 28.4 | 19.8 |
| 29.2 | 19.5 |
| 30.2 | 20.5 |
| 32.1 | 16.9 |
| 32.6 | 20.2 |
| 32.9 | 28.4 |
| 33.2 | 16.6 |
| 34.6 | 15.4 |

Reflections having the greatest relative intensity at 7.1, 8.0, 10.5, 16.0, and 21.5 degrees two-theta for the mesylate salt Form B. Superscript (u) denotes unique reflections of Form C.

TABLE 6
List of powder x-ray diffraction reflections for the besylate
salt Form A.

| Angle | Intensity |
|---|---|
| 2-Theta ± 0.2° | Percent |
| 6.8 | 11.8 |
| 7.7 | 100 |
| 12.2 | 13.9 |
| 13.4 | 12.9 |
| 15.4 | 75.2 |
| 20.3 | 14.1 |
| 20.6 | 8.8 |
| 22.0 | 11.2 |
| 22.6 | 31.9 |
| 23.0 | 15.6 |
| 23.7 | 50.6 |
| 24.1 | 51.8 |
| 24.6 | 13.7 |
| 25.1 | 17.4 |
| 26.5 | 18.8 |
| 27.0 | 11.3 |
| 27.9 | 37.2 |
| 29.0 | 16.3 |
| 31.1 | 12.1 |

Reflections having the greatest relative intensity at 7.7, 15.4, 23.7, 24.1, and 27.9 degrees two-theta for the mesylate salt Form A.

TABLE 7
List of powder x-ray diffraction reflections for the tosylate
salt Form A.

| Angle | Intensity |
|---|---|
| 2-Theta ± 0.2° | Percent |
| 6.7 | 6.5 |
| 7.4 | 100 |
| 11.9 | 6.8 |
| 13.4 | 8 |
| 14.8 | 53.6 |
| 16.2 | 2.6 |
| 19.7 | 4.7 |
| 20.2 | 4.3 |
| 21.6 | 4.3 |
| 22.0 | 5.4 |
| 22.8 | 13.4 |
| 23.2 | 19.7 |
| 24.1 | 33 |
| 26.9 | 8.3 |
| 27.9 | 11.6 |
| 28.6 | 8.8 |
| 29.5 | 5.8 |
| 29.8 | 10 |

Reflections having the greatest relative intensity at 7.4, 11.9, 14.8, 22.8, 23.2, and 24.1 degrees two-theta for the tosylate salt Form A.

### Method for Collecting Differential Scanning Calorimetry Data for 1-[5-(4-amino-7-isopropyl-7H-pyrrolo[2,3-d]pyrimidine-5-carbonyl)-2-methoxy-phenyl]-3-(2,4-dichloro-phenyl)-urea salts

Thermal phase transition data was collected using a TA Instruments (New Castle, Del.) differential scanning calorimeter Q1000. Calibration of the temperature axis and cell constant was accomplished with Indium (about 5 mg, 99.99 percent pure, peak maximum at 156.6° C., heat of fusion of 28.4 J/g). Crimped aluminum sample pans with a pinhole in the lid were loaded with one to two milligrams of sample and then scanned from room temperature to 300° C. at 5° C./minute. Calibration and sample analysis utilized empty aluminum sample pans as a reference and a dry nitrogen purge gas flow rate of 50 mL/minute. Onset temperatures are determined by the baseline tangent-peak tangent method.

TABLE 8
Differential scanning calorimetry analysis.

| | A. Solid-State | Onset Temperature |
|---|---|---|
| Anion | Form | (±2° C.) |
| Phosphoric Acid | Form A | 202° C. |
| Methane sulfonic acid | Form A | 202° C. |
| Methane sulfonic acid | Form B | 195° C. |
| Methane sulfonic acid | Form C | 50-70° C. |
| hemihydrate | | 168° C., 190° C. |
| Benzene sulfonic acid | Form A | 250° C. |
| Toluene sulfonic acid | Form A | 255° C. |

1. A pharmaceutical composition comprising a solid amorphous dispersion of 1-[5-(4-amino-7-isopropyl-7H-pyrrolo[2,3-d]pyrimidine-5-carbonyl)-2-methoxy-phenyl]-3-(2,4-dichloro-phenyl)-urea and a concentration enhancing polymer.

2. A pharmaceutical composition comprising a solid amorphous dispersion of 1-[5-(4-amino-7-isopropyl-7H-pyrrolo[2,3-d]pyrimidine-5-carbonyl)-2-methoxy-phenyl]-3-(2,4-dichloro-phenyl)-urea and a concentration-enhancing polymer, wherein said 1-[5-(4-amino-7-isopropyl-7H-pyrrolo[2,3-d]pyrimidine-5-carbonyl)-2-methoxy-phenyl]-3-(2,4-dichloro-phenyl)-urea comprises between 10 to 40 percent by weight of said solid amorphous dispersion.

3. A pharmaceutical composition comprising a solid amorphous dispersion of 1-[5-(4-amino-7-isopropyl-7H-pyrrolo[2,3-d]pyrimidine-5-carbonyl)-2-methoxy-phenyl]-3-(2,4-dichloro-phenyl)-urea and a concentration-enhancing polymer, wherein said 1-[5-(4-amino-7-isopropyl-7H-pyrrolo[2,3-d]pyrimidine-5-carbonyl)-2-methoxy-phenyl]-3-(2,4-dichloro-phenyl)-urea is between 10 to 40 percent by weight of said solid amorphous dispersion, and wherein said concentration-enhancing polymer is a cellulosic.

4. A pharmaceutical composition comprising a solid amorphous dispersion of 1-[5-(4-amino-7-isopropyl-7H-pyrrolo[2,3-d]pyrimidine-5-carbonyl)-2-methoxy-phenyl]-3-(2,4-dichloro-phenyl)-urea and a concentration-enhancing polymer, wherein said 1-[5-(4-amino-7-isopropyl-7H-pyrrolo[2,3-d]pyrimidine-5-carbonyl)-2-methoxy-phenyl]-3-(2,4-dichloro-phenyl)-urea is between 10 to 40 percent by weight of said solid amorphous dispersion, and wherein said concentration-enhancing polymer is hydroxypropyl methylcellulose acetate succinate.

5. The pharmaceutical composition of any one of the previous claims wherein said 1-[5-(4-Amino-7-isopropyl-7H-pyrrolo[2,3-d]pyrimidine-5-carbonyl)-2-methoxy-phenyl]-3-(2,4-dichloro-phenyl)-urea is substantially amorphous and said dispersion is substantially homogeneous.

6. The pharmaceutical composition of any one of the previous claims wherein said dispersion has a single glass transition temperature.

7. A pharmaceutical composition comprising a solid amorphous dispersion of 1-[5-(4-amino-7-isopropyl-7H-pyrrolo[2,3-d]pyrimidine-5-carbonyl)-2-methoxy-phenyl]-3-(2,4-dichloro-phenyl)-urea and a concentration-enhancing polymer, wherein said concentration-enhancing polymer is present in said solid amorphous dispersion in a sufficient amount so that said composition provides concentration enhancement of said 1-[5-(4-amino-7-isopropyl-7H-pyrrolo[2,3-d]pyrimidine-5-carbonyl)-2-methoxy-phenyl]-3-(2,4-dichloro-phenyl)-urea in a use environment relative to a control composition consisting essentially of an equivalent amount of said 1-[5-(4-amino-7-isopropyl-7H-pyrrolo[2,3-d]pyrimidine-5-carbonyl)-2-methoxy-phenyl]-3-(2,4-dichloro-phenyl)-urea alone, and wherein said concentration-enhancing polymer is hydroxypropyl methylcellulose acetate succinate.

8. A pharmaceutical composition of any one of the previous claims wherein said concentration-enhancing polymer composition when administered at least once during a 24 hour period in an oral dosage form of between 5 mg and 500 mg of 1-[5-(4-amino-7-isopropyl-7H-pyrrolo[2,3-d]pyrimidine-5-carbonyl)-2-methoxy-phenyl]-3-(2,4-dichloro-phenyl)-urea to a human, has a Cₘₐₓ plasma level as determined in a fasting rat at a dose of 100 mg 1-[5-(4-amino-7-isopropyl-7H-pyrrolo[2,3-d]pyrimidine-5-carbonyl)-2-methoxy-phenyl]-3-(2,4-dichloro-phenyl)-urea per kg, between 20000 ng base/ml to 1000 ng base/ml over said 24 hour period.

9. A pharmaceutical composition of any one of the previous claims wherein said concentration-enhancing polymer composition when administered at least once during a 24 hour period in an oral dosage form of between 5 mg and 500 mg of 1-[5-(4-amino-7-isopropyl-7H-pyrrolo[2,3-d]pyrimidine-5-carbonyl)-2-methoxy-phenyl]-3-(2,4-dichloro-phenyl)-urea to a human, has a Cₘₐₓ plasma level as determined in a fasting dog at a dose of 30 mg 1-[5-(4-amino-7-isopropyl-7H-pyrrolo[2,3-d]pyrimidine-5-carbonyl)-2-methoxy-phenyl]-3-(2,4-dichloro-phenyl)-urea per kg, between 15000 ng base/ml to 1000 ng base/ml over said 24 hour period.

10. A pharmaceutical composition of any one of the previous claims wherein said concentration-enhancing polymer composition when administered at least once during a 24 hour period in an oral dosage form of between 5 mg and 500 mg of 1-[5-(4-amino-7-isopropyl-7H-pyrrolo[2,4-d]pyrimidine-5-carbonyl)-2-methoxy-phenyl]-3-(2,4-dichloro-phenyl)-urea to a human, has a AUC₀₋₂₄ plasma level as determined in a fasting rat at a dose of 100 mg 1-[5-(4-amino-7-isopropyl-7H-pyrrolo[2,3-d]pyrimidine-5-carbonyl)-2-methoxy-phenyl]-3-(2,4-dichloro-phenyl)-urea per kg, between 150000 ng base×hr/mL and 5000 ng base×hr/mL.

11. A pharmaceutical compositions of any one of the previous claims wherein said concentration-enhancing polymer composition when administered at least once during a 24 hour period in an oral dosage form of between 5 mg and 500 mg of 1-[5-(4-amino-7-isopropyl-7H-pyrrolo[2,3-d]pyrimidine-5-carbonyl)-2-methoxy-phenyl]-3-(2,4-dichloro-phenyl)-urea to a human, has a AUC₀₋₂₄ plasma level as determined in a fasting dog at a dose of 30 mg 1-[5-(4-amino-7-isopropyl-7H-pyrrolo[2,3-d]pyrimidine-5-carbonyl)-2-methoxy-phenyl]-3-(2,4-dichloro-phenyl)-urea per kg, between 100000 ng base×hr/mL and 8000 ng base×hr/mL.

12. A pharmaceutical composition of any one of the previous claims wherein said concentration-enhancing polymer composition when administered at least once during a 24 hour period in an oral dosage form of between 5 mg and 500 mg of 1-[5-(4-amino-7-isopropyl-7H-pyrrolo[2,3-d]pyrimidine-5-carbonyl)-2-methoxy-phenyl]-3-(2,4-dichloro-phenyl)-urea to a human, has a Tₘₐₓ plasma level as determined in a fasting rat at a dose of 100 mg 1-[5-(4-amino-7-isopropyl-7H-pyrrolo[2,3-d]pyrimidine-5-carbonyl)-2-methoxy-phenyl]-3-(2,4-dichloro-phenyl)-urea per kg, less then 3 hours and 30 minutes.

13. A pharmaceutical composition of any one of the previous claims wherein said concentration-enhancing polymer composition when administered at least once during a 24 hour period in an oral dosage form of between 5 mg and 500 mg of 1-[5-(4-amino-7-isopropyl-7H-pyrrolo[2,3-d]pyrimidine-5-carbonyl)-2-methoxy-phenyl]-3-(2,4-dichloro-phenyl)-urea to a human, has a Tₘₐₓ plasma level as determined in a fasting dog at a dose of 30 mg 1-[5-(4-amino-7-isopropyl-7H-pyrrolo[2,3-d]pyrimidine-5-carbonyl)-2-methoxy-phenyl]-3-(2,4-dichloro-phenyl)-urea per kg, less then 3 hours and 30 minutes.

14. The pharmaceutical composition of any one of the previous claims wherein said solid amorphous dispersion is mixed with additional concentration-enhancing polymer.

15. The pharmaceutical composition of any one of the previous claims wherein said concentration-enhancing polymer comprises a blend of polymers.

16. The pharmaceutical composition of any one of the previous claims wherein said concentration-enhancing polymer has at least one hydrophobic portion and at least one hydrophilic portion.

17. The pharmaceutical composition of any one of the previous claims wherein said concentration-enhancing polymer is selected from the group consisting of ionizable cellulosic polymers, nonionizable cellulosic polymers, and vinyl copolymers and copolymers having substituents selected from the group consisting of hydroxyl, alkylacyloxy, and cyclicamido.

18. The pharmaceutical composition of any one of the previous claims wherein said concentration-enhancing polymer is selected from the group consisting of hydroxypropyl methyl cellulose acetate, hydroxypropyl methyl cellulose, hydroxypropyl cellulose, methyl cellulose, hydroxyethyl methyl cellulose, hydroxyethyl cellulose acetate, hydroxyethyl ethyl cellulose, hydroxypropyl methyl cellulose acetate succinate, cellulose acetate phthalate, hydroxypropyl methyl cellulose phthalate, methyl cellulose acetate phthalate, cellulose acetate trimellitate, hydroxypropyl cellulose acetate phthalate, cellulose acetate terephthalate, cellulose acetate isophthalate, and carboxy methyl ethyl cellulose.

19. The pharmaceutical composition of any one of the previous claims wherein said solid amorphous dispersion is formulated in a tablet.

20. The pharmaceutical composition according to any one of the previous claims, further comprising a disintegrant.

21. The pharmaceutical composition according to claim 20, wherein said disintegrant is selected from the group consisting of sodium starch glycolate, sodium alginate, carboxy methyl cellulose sodium, methyl cellulose, and croscarmellose sodium.

22. The pharmaceutical composition according to any one of the previous claims, further comprising a binder.

23. The pharmaceutical composition according to claim 22, wherein said binder is selected from the group consisting of methyl cellulose, microcrystalline cellulose, starch, and gums.

24. The pharmaceutical composition according to any one of the previous claims, further comprising a lubricant.

25. The pharmaceutical composition according to claim 24, wherein said lubricant is selected from the group consisting of magnesium stearate and calcium stearate.

26. The pharmaceutical composition of any one of the previous claims wherein said solid amorphous dispersion is formulated in a capsule.

27. A method for the treatment of abnormal cell growth in a mammal in need of such treatment comprising administering to said mammal an amount of a solid amorphous dispersion of 1-[5-(4-amino-7-isopropyl-7H-pyrrolo[2,3-d]pyrimidine-5-carbonyl)-2-methoxy-phenyl]-3-(2,4-dichloro-phenyl)-urea and a concentration enhancing polymer.

28. A method for the treatment of abnormal cell growth in a mammal in need of such treatment comprising administering to said mammal an amount of a solid amorphous dispersion of 1-[5-(4-amino-7-isopropyl-7H-pyrrolo[2,3-d]pyrimidine-5-carbonyl)-2-methoxy-phenyl]-3-(2,4-dichloro-phenyl)-urea and a concentration-enhancing polymer, wherein said concentration-enhancing polymer comprises between 10 to 40 percent by weight of said solid amorphous dispersion.

29. A method for the treatment of abnormal cell growth in a mammal in need of such treatment comprising administering to said mammal an amount of a solid amorphous dispersion of 1-[5-(4-amino-7-isopropyl-7H-pyrrolo[2,3-d]pyrimidine-5-carbonyl)-2-methoxy-phenyl]-3-(2,4-dichloro-phenyl)-urea and a concentration-enhancing polymer, wherein said concentration-enhancing polymer is between 10 to 40 percent by weight of said solid amorphous dispersion, and wherein said concentration-enhancing polymer is a cellulosic.

30. A method for the treatment of abnormal cell growth in a mammal in need of such treatment comprising administering to said mammal an amount of a solid amorphous dispersion of 1-[5-(4-amino-7-isopropyl-7H-pyrrolo[2,3-d]pyrimidine-5-carbonyl)-2-methoxy-phenyl]-3-(2,4-dichloro-phenyl)-urea and a concentration-enhancing polymer, wherein said concentration-enhancing polymer is between 10 to 40 percent by weight of said solid amorphous dispersion, and wherein said concentration-enhancing polymer is hydroxypropyl methylcellulose acetate succinate.

31. A method of treatment according to any one of claims 27-30 wherein said 1-[5-(4-amino-7-isopropyl-7H-pyrrolo[2,3-d]pyrimidine-5-carbonyl)-2-methoxy-phenyl]-3-(2,4-dichloro-phenyl)-urea is substantially amorphous and said dispersion is substantially homogeneous.

32. A method of treatment according to any one of claims 27-31 wherein said dispersion has a single glass transition temperature.

33. A method for the treatment of abnormal cell growth in a mammal in need of such treatment comprising administering to said mammal an amount of a solid amorphous dispersion of 1-[5-(4-amino-7-isopropyl-7H-pyrrolo[2,3-d]pyrimidine-5-carbonyl)-2-methoxy-phenyl]-3-(2,4-dichloro-phenyl)-urea and a concentration-enhancing polymer, wherein said concentration-enhancing polymer is present in said solid amorphous dispersion in a sufficient amount so that said composition provides concentration enhancement of said 1-[5-(4-amino-7-isopropyl-7H-pyrrolo[2,3-d]pyrimidine-5-carbonyl)-2-methoxy-phenyl]-3-(2,4-dichloro-phenyl)-urea in a use environment relative to a control composition consisting essentially of an equivalent amount of said 1-[5-(4-amino-7-isopropyl-7H-pyrrolo[2,3-d]pyrimidine-5-carbonyl)-2-methoxy-phenyl]-3-(2,4-dichloro-phenyl)-urea alone, and where in said concentration-enhancing polymer is hydroxypropyl methylcellulose acetate succinate.

34. A method of treatment according to any one of claims 27-33, wherein said solid amorphous dispersion is mixed with additional concentration-enhancing polymer.

35. A method of treatment according to any one of claims 27-34, wherein said concentration-enhancing polymer comprises a blend of polymers.

36. A method of treatment according to any one of claims 27-35, wherein said concentration-enhancing polymer has at least one hydrophobic portion and at least one hydrophilic portion.

37. A method of treatment according to any one of claims 27-36, wherein said concentration-enhancing polymer is selected from the group consisting of ionizable cellulosic polymers, nonionizable cellulosic polymers, and vinyl copolymers and copolymers having substituents selected from the group consisting of hydroxyl, alkylacyloxy, and cyclicamido.

38. A method of treatment according to any one of the claims 27-37, wherein said concentration-enhancing polymer is selected from the group consisting of hydroxypropyl methyl cellulose acetate, hydroxypropyl methyl cellulose, hydroxypropyl cellulose, methyl cellulose, hydroxyethyl methyl cellulose, hydroxyethyl cellulose acetate, hydroxyethyl ethyl cellulose, hydroxypropyl methyl cellulose acetate succinate, cellulose acetate phthalate, hydroxypropyl methyl cellulose phthalate, methyl cellulose acetate phthalate, cellulose acetate trimellitate, hydroxypropyl cellulose acetate phthalate, cellulose acetate terephthalate, cellulose acetate isophthalate, and carboxy methyl ethyl cellulose.

39. A method according to claims 27-38, wherein said abnormal cell growth is cancer.

40. A method for the treatment of a hyperproliferative disorder in a mammal which comprises administering to said mammal a therapeutically effective amount of 1-[5-(4-amino-7-isopropyl-7H-pyrrolo[2,3-d]pyrimidine-5-carbonyl)-2-methoxy-phenyl]-3-(2,4-dichloro-phenyl)-urea in combination with one to three anti-tumor agents selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.
